(19) United States
(12) Patent Application Publication
(10) Pub. No.: US 2024/0108710 A1
(43) Pub. Date: Apr. 4, 2024

(54) NANOEMULSION ADJUVANT COMPOSITION FOR PNEUMOCOCCAL CONJUGATE VACCINES

(71) Applicant: Merck Sharp & Dohme LLC, Rahway, NJ (US)

(72) Inventors: William J. Smith, Harleysville, PA (US); Patrick L. Ahl, Yardley, PA (US); Randal J. Soukup, Hatfield, PA (US); Julie M. Skinner, Phoenixville, PA (US)

(73) Assignee: Merck Sharp & Dohme LLC, Rahway, NJ (US)

(21) Appl. No.: 18/263,289

(22) PCT Filed: Feb. 2, 2022
(86) PCT No.: PCT/US2022/014812

§ 371 Date: Jul. 27, 2023

Related U.S. Application Data
(60) Provisional application No. 63/145,651, filed on Feb. 4, 2021.

(51) Int. Cl. A61K 39/09; A61K 47/64

(52) U.S. Cl. CPC A61K 39/092; A61K 47/646; A61K 2039/55566

(57) ABSTRACT

The present invention relates generally to the prevention of pneumococcal disease. More specifically, the invention relates to a composition comprising pneumococcal conjugates and a stable nanoemulsion (SNE).

### BACKGROUND OF THE INVENTION

Pneumococcal disease is an infection caused by the bacteria Streptococcus pneumoniae (pneumococcus). Different pneumococcal serotypes are known to cause different manifestations of the disease and infections can cause a range of symptoms from ear and sinus infections to pneumonia and bloodstream infections. Pneumococcal disease has a high associated morbidity and mortality worldwide, particularly among the elderly and young children. Currently 100 capsular polysaccharides have been identified (Ganaie, F. et al. (2020) Clinical Science and Epidemiology, Vol. 11, Issue 3, pages 1-15). These serotypes are distinguished by their chemical structure, serological response, and other related genetic mutations.

In 1983, PNEUMOVAX® (Merck Sharp & Dohme Corp., a subsidiary of Merck & Co., Inc., Kenilworth, NJ, USA), a 23-valent pneumococcal vaccine was approved in the United States. This vaccine demonstrated reduced immunogenicity in infants due to T-cell independent responses. To address this issue, particularly in infants, pneumococcal conjugate vaccines (PCVs) were developed by covalently coupling the polysaccharide to a carrier protein, wherein the immunogenic response became T-cell dependent. In 2000, PREVNAR® (Wyeth Pharmaceuticals LLC, Collegeville, PA), a 7-valent pneumococcal conjugate vaccine was approved in the United States. In 2010, PREVNAR13@ (Wyeth Pharmaceuticals LLC, Collegeville, PA), a 13-valent pneumococcal conjugate vaccine was approved in the United States. In 2021, VAXNEUVANCE® (Merck Sharp & Dohme Corp., a subsidiary of Merck & Co., Inc., Kenilworth, NJ, USA), a 15-valent pneumococcal conjugate vaccine and PREVNAR20@ (Wyeth Pharmaceuticals LLC, Collegeville, PA), a 20-valent pneumococcal conjugate vaccine were also approved in the United States. Other multivalent PCVs are known and licensed worldwide.

Licensed PCVs currently utilize aluminum containing derivatives as adjuvants to increase immunogenicity. Even though aluminum adjuvants increase immunogenic responses from baseline, it is unknown whether the immunogenic response is sufficient for higher valency PCVs, particularly in infants. Therefore, there is a need to identify other adjuvants that can provide increased immunogenicity for multivalent PCVs over the current aluminum adjuvant standard(s).

### SUMMARY OF THE INVENTION

The present invention relates generally to the prevention of pneumococcal disease. More specifically, the invention relates to compositions administered as a vaccine which include pneumococcal conjugates and a stable nanoemulsion (SNE) adjuvant formulation. The present disclosure provides, among other things, a pneumococcal conjugate composition including an SNE comprising emulsifiers and/or solubilizers and/or surfactants and/or lipids. The present disclosure provides, among other things, a pneumococcal conjugate composition including an SNE comprising surfactants and/or terpenes and/or cationic lipids or mixtures thereof. The present disclosure provides, among other things, a pneumococcal conjugate composition including an SNE comprising sorbitan esters and/or terpenes and/or cationic lipids or mixtures thereof. The present disclosure provides, among other things, a pneumococcal conjugate composition including an SNE comprising sorbitan esters, in particular polysorbate-20 or polysorbate-80 or a poloxamer and/or terpenes and/or cationic lipids or mixtures thereof. The present disclosure provides, among other things, a pneumococcal conjugate composition including an SNE comprising sorbitan esters, in particular polysorbate-20 or polysorbate-80 or a poloxamer and/or terpenes and/or cationic lipids or mixtures thereof. The present disclosure provides, among other things, a pneumococcal conjugate composition including an SNE comprising sorbitan trioleate (SPAN-85), polysorbate-20 or polysorbate-80, a terpene and an optional cationic lipid. The present disclosure provides, among other things, a pneumococcal conjugate composition including an SNE comprising sorbitan trioleate (SPAN-85), polysorbate-20 or polysorbate-80, squalene and an optional cationic lipid. The present disclosure provides, among other things, a pneumococcal conjugate composition including an SNE comprising sorbitan trioleate (SPAN-85), polysorbate-20 or polysorbate-80, squalene and a cationic lipid. The present disclosure further provides, among other things, a pneumococcal conjugate composition including an SNE adjuvant formulation comprising 1) sorbitan trioleate (SPAN-85); 2) polysorbate-20 (PS-20) or polysorbate-80 (PS-80); 3) squalene; and an optional 4) cationic lipid. A particular pneumococcal conjugate composition includes an SNE adjuvant formulation comprising 1) sorbitan trioleate (SPAN-85); 2) polysorbate-20 (PS-20) or polysorbate-80 (PS-80); 3) squalene; and 4) the cationic lipid (13Z,16Z)—N, N-dimethyl-3-nonyldocosa-13,16-dien-1-amine (“CLA” or, when the cationic lipid is included in the SNE, “CLA-SNE”). A particular pneumococcal conjugate composition includes an SNE adjuvant formulation comprising 1) sorbitan trioleate (SPAN-85); 2) polysorbate-20 (PS-20) or polysorbate-80 (PS-80); 3) squalene; and 4) the cationic lipid (13Z,16Z)—N, N-dimethyl-3-nonyldocosa-13,16-dien-1-amine (“CLA” or, when the cationic lipid is included in the SNE, “CLA-SNE”). A particular pneumococcal conjugate composition includes an SNE adjuvant formulation comprising 1) sorbitan trioleate (SPAN-85); 2) polysorbate-20 (PS-20) or polysorbate-80 (PS-80); 3) squalene; and 4) the cationic lipid (13Z,16Z)—N, N-dimethyl-3-nonyldocosa-13,16-dien-1-amine (“CLA” or, when the cationic lipid is included in the SNE, “CLA-SNE”). A particular pneumococcal conjugate composition includes an SNE adjuvant formulation comprising 1) sorbitan trioleate (SPAN-85); 2) polysorbate-20 (PS-20); 3) squalene; and 4) the cationic lipid (13Z,16Z)—N, N-dimethyl-3-nonyldocosa-13,16-dien-1-amine (“CLA” or, when the cationic lipid is included in the SNE, “CLA-SNE”). Compared to the performance of 1) a non-adjuvanted pneumococcal conjugate composition; 2) an aluminum phosphate adjuvanted (APA) pneumococcal conjugate composition; or 3) an LNP adjuvanted pneumococcal conjugate composition; the described SNE adjuvant pneumococcal conjugate composition provided an equivalent or increased immunogenic response for the majority of pneumococcal serotypes tested.

### BRIEF DESCRIPTION OF THE DRAWINGS

FIG. 1: Select structures of cationic lipids: (13Z,16Z)—N, N-dimethyl-3-nonyldocosa-13,16-dien-1-amine (CLA); (6Z,9Z,26Z,29Z)—N,N-dimethylpentatriaconta-6,9,26,29-tetraen-18-amine (CLX); and N,N-dimethyl-1-((1S,2R)-2-octylcyclopropyl) heptadecan-8-amine (CLY).

FIG. 2: CLA-SNE components: (13Z,16Z)—N, N-dimethyl-3-nonyldocosa-13,16-dien-1-amine (CLA), SPAN-85, PS-20 and squalene.

FIG. 3: Characterization of CLA-SNE adjuvant bulk preparation utilizing static light scattering (SLS). See Example 3.

FIG. 4: Impact of the formulation process on the incorporation of CLA into an SNE. See Example 4.

FIG. 5A: Pre-immune (pooled) and post-dose 3 (day 35) anti-6B IgG titers after immunization of mice with formulations described in Table 4. Error bars are geometric means with 95% confidence intervals. Transformed data analyzed by one-way ANOVA with Dunnett post-test *p<0.05, **p<0.01, ***p<0.001, ****p<0.0001, NS is not significant. See Example 6.

FIG. 5B: Pre-immune (pooled) and post-dose 3 (day 35) (pooled) serotype 6B opsonophagocytic killing titers of mice immunized with formulations described in Table 4. See Example 6.

FIG. 6A: Ratio of serotype specific IgG titers in adult rhesus macaques following immunization with PCV24 formulated with CLA-SNE compared to PCV24 formulated with APA at post dose 1 (PD1: circles) and post dose 2 (PD2: squares). PCV24 formulated with CLA-SNE (1200 μg/mL CLA-SNE) results in equal or better immunogenicity as compared to PCV24 formulated with APA. Serotype 6C and 15B data are included to evaluate cross reactivity. See Example 7.

FIG. 6B: Ratio of serotype specific IgG titers in adult rhesus macaques following immunization with formulations described in Table 5 compared to PCV24 formulated with APA at post dose 2 (PD2). PCV24 formulated with CLA-SNE (circles and triangles), CLA-LNP (squares), or SNE (diamonds) results in equal or better immunogenicity as compared to PCV24 formulated with APA. Serotype 6C and 15B data are included to evaluate cross reactivity. See Example 7.

FIG. 7A: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 1 and 3. See Example 7.

FIG. 7B: Pre-immune (individual/pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 4 and 5. See Example 7.

FIG. 7C: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 6A and 6B. See Example 7.

FIG. 7D: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 6C and 7F. See Example 7.

FIG. 7E: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 8 and 9V. See Example 7.

FIG. 7F: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 10A and 11A. See Example 7.

FIG. 7G: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 12F and 14. See Example 7.

FIG. 7H: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 15A and 15C. See Example 7.

FIG. 7I: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 18C and 19A. See Example 7.

FIG. 7J: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 19F and 22F. See Example 7.

FIG. 7K: Pre-immune (pooled/individual), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 23B and 23F. See Example 7.

FIG. 7L: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotypes 24F and 33F. See Example 7.

FIG. 7M: Pre-immune (pooled), post dose 1 (day 14) and post dose 2 (day 42) opsonophagocytic killing titers of adult rhesus macaques following immunization with formulations described in Table 5. Dose volume administered is 0.1 mL per animal and results in a 0.08 mg or 0.12 mg delivered dose of CLA for groups PCV24/CLA-SNE (80 μg) and PCV24/CLA-SNE (120 μg), respectively. Rhesus sera were evaluated for functional antibodies determined through multiplexed opsonophagocytic assays (MOPA) for serotype 35B. See Example 7.

FIG. 8A: Ratio of serotype specific IgG titers in infant rhesus macaques following immunization with PCV13, PCV24 with CLA-LNP (120 μg dose), PCV24 with CLA-SNE (295 μg CLA & 2.5 mg squalene), PCV24 with CLA-SNE (295 μg CLA & 0.5 mg squalene) compared to an immunization of PCV24 formulated with APA at post dose 2 (day 42). Serotypes 6C and 15B data are included to evaluate cross reactivity. See Example 8.

FIG. 8B: Ratio of serotype specific IgG titers in infant rhesus macaques following immunization with select formulations described in Table 6 compared to PCV24 formulated with APA at post dose 1 (day 14). Serotypes 6C and 15B data are included to evaluate cross reactivity. See Example 8.

FIG. 8C: Ratio of serotype specific IgG titers in infant rhesus macaques following immunization with select formulations described in Table 6 compared to PCV24 formulated with APA at post dose 2 (day 42). Serotypes 6C and 15B data are included to evaluate cross reactivity. See Example 8.

FIG. 8D: Ratio of serotype specific IgG titers in infant rhesus macaques following immunization with select formulations described in Table 6 compared to PCV24 formulated with APA at post dose 3 (day 70). Serotypes 6C and 15B data are included to evaluate cross reactivity. See Example 8.

FIG. 9: PCV24 immunized mice with formulation compositions (CLA-SNE; CLA-LNP; and SNE) are protected from Streptococcus pneumoniae serotype 24F intratracheal challenge. See Example 9.

FIG. 10A-10D: Nanotracking analysis (NTA) of CLA-SNE and SNE formulations stored at 4° C. and 37° C. for 1 month (FIG. 10A: CLA-SNE [6 mg/mL CLA and 30 mg/mL squalene]; FIG. 10B: SNE [40 mg/mL squalene]; FIG. 10C: CLA-SNE [4 mg/mL CLA and 4 mg/mL squalene]; and FIG. 10D: SNE [8 mg/mL squalene]). See Example 10.

FIG. 11A-11D: Dynamic light scattering (DLS) of CLA-SNE and SNE formulations stored at 4° C., 25° C. and 37° C. for 1 month (FIG. 11A: CLA-SNE [6 mg/mL CLA and 30 mg/mL squalene]; FIG. 11B: CLA-SNE [4 mg/mL CLA and 4 mg/mL squalene]; FIG. 11C: SNE [40 mg/mL squalene]; and FIG. 11D: SNE [8 mg/mL squalene]). See Example 10.

FIG. 12A: CLA concentration (mg/mL) as measured by UPLC-CAD for CLA-SNE and SNE formulations stored at 4° C., 25° C. and 37° C. for 1 month. See Example 11.

FIG. 12B: Squalene concentration (mg/mL) as measured by UPLC-CAD for CLA-SNE and SNE formulations stored at 4° C., 25° C. and 37° C. for 1 month. See Example 11.

FIG. 13A: Serotype specific stability of pneumococcal polysaccharide-carrier protein conjugate coformulations prepared with CLA-SNE (1.2 mg/mL CLA 6.5 mg/mL squalene) and stored at 4° C. for 1 month. See Example 12.

FIG. 13B: Serotype specific stability of pneumococcal polysaccharide-carrier protein conjugate coformulations prepared with CLA-SNE (1.2 mg/mL CLA 1.2 mg/mL squalene) and stored at 4° C. for up to 1 month. See Example 12.

FIG. 13C: Serotype specific stability of pneumococcal polysaccharide-carrier protein conjugate coformulations prepared with SNE (6.5 mg/mL squalene) and stored at 4° C. for 1 month. See Example 12.

FIG. 13D: Serotype specific stability of pneumococcal polysaccharide-carrier protein conjugate coformulations prepared with SNE (0.4 mg/mL squalene) and stored at 4° C. for 1 month. See Example 12.

FIG. 14A: Ratio of serotype specific IgG titers in adult rhesus macaques following immunization with PCV21 formulated with CLA-SNE compared to PCV21 (no adjuvant) at post dose 1 (Day 14) [referred to as D14PD1: squares]. A 0.25 mL dose of PCV21 (4 μg/mL per ST) formulated with CLA-SNE (1200 μg/mL CLA-SNE) results in equal or better immunogenicity as compared to a 0.25 mL dose of PCV21 (4 μg/mL per ST) at D14PD1. Serotype 6C and 15B data are included to evaluate cross reactivity. See Example 13.

FIG. 14B: Ratio of serotype specific IgG titers in adult rhesus macaques following immunization with PCV21 formulated with CLA-SNE compared to PCV21 (no adjuvant) at post dose 1 (Day 28) [referred to as D28PD1: squares]. A 0.25 mL dose of PCV21 (4 μg/mL per ST) formulated with CLA-SNE (1200 μg/mL CLA-SNE) results in equal or better immunogenicity as compared to a 0.25 mL dose of PCV21 (4 μg/mL per ST) at D28PD1. Serotype 6C and 15B data are included to evaluate cross reactivity. See Example 13.

FIG. 14C: Ratio of serotype specific IgG titers in adult rhesus macaques following immunization with PCV21 formulated with CLA-SNE compared to PCV21 (no adjuvant) on post dose 2 (Day 42) [referred to as D42PD2: squares]. A 0.25 mL dose of PCV21 (4 μg/mL per ST) formulated with CLA-SNE (1200 μg/mL CLA-SNE) results in equal or better immunogenicity as compared to a 0.25 mL dose of PCV21 (4 μg/mL per ST) at D42PD2. Serotype 6C and 15B data are included to evaluate cross reactivity. See Example 13.

FIG. 15A: The CLA/squalene (w/w) % after dialysis is plotted versus the “target” (w/w) % before self-assembly. The CLA/squalene w/w % ratios (X) were measured by reverse phase UPLC-CAD before and after self-assembly and nanoemulsion dialysis. See Example 14.

FIG. 15B: The measured intensity weighted Z-average DLS diameters of CLA-SNE nanoparticles after dialysis (X) is plotted versus the measured CLA/squalene (w/w) % after dialysis for each of MNS formulation. See Example 14.

FIG. 15C: The measured Zeta Potential of CLA-SNE squalene nanoparticles (X) after dialysis at pH 5.5 is plotted versus the measured CLA/squalene (w/w) % after dialysis for each of MNS prepared formulations. See Example 14.

FIG. 16. DLS Z-averages diameters of CLA-SNE samples formed and processed with aqueous phase (20 mM L-Histidine) of increasing pH values. See Example 15.

FIG. 17. Final [CLA] (mg/mL) of CLA-SNE samples formed and processed with aqueous phase (20 mM L-Histidine) of increasing pH values. See Example 15.

### DETAILED DESCRIPTION OF THE INVENTION

It was surprisingly found that a pneumococcal conjugate composition comprising a stable nanoemulsion (SNE) adjuvant formulation (with or without a cationic lipid) provided a comparable or enhanced immunogenic response relative to a pneumococcal conjugate composition comprising an aluminum adjuvant and/or a pneumococcal conjugate composition comprising an LNP adjuvant.

The present invention provides a pneumococcal conjugate composition comprising Streptococcus pneumoniae polysaccharide-carrier protein conjugates and a stable nanoemulsion (SNE).

The present invention further provides a pneumococcal conjugate composition comprising Streptococcus pneumoniae polysaccharide-carrier protein conjugates and an SNE, and a pharmaceutically acceptable carrier.

The present invention further provides a pneumococcal conjugate composition comprising Streptococcus pneumoniae polysaccharide-carrier protein conjugates and an SNE comprising an emulsifier and/or a solubilizer and/or a surfactant and optionally a cationic lipid, or mixtures thereof.

The present invention further provides a pneumococcal conjugate composition comprising Streptococcus pneumoniae polysaccharide-carrier protein conjugates and an SNE comprising surfactants and/or terpenes or mixtures thereof.

The present invention further provides a pneumococcal conjugate composition comprising Streptococcus pneumoniae polysaccharide-carrier protein conjugates and an SNE comprising surfactants and/or terpenes and/or cationic lipids or mixtures thereof.

The present invention further provides a pneumococcal conjugate composition comprising Streptococcus pneumoniae polysaccharide-carrier protein conjugates and an SNE comprising one or more surfactants and one or more terpenes and optionally one or more cationic lipids.

The present invention further provides a pneumococcal conjugate composition comprising Streptococcus pneumoniae polysaccharide-carrier protein conjugates and an SNE comprising one to three surfactants and one to three terpenes and optionally one to three cationic lipids.

The present invention further provides a pneumococcal conjugate composition comprising Streptococcus pneumoniae polysaccharide-carrier protein conjugates and an SNE comprising one to two surfactants and one to two terpenes and optionally one to two cationic lipids.

The present invention further provides a pneumococcal conjugate composition comprising Streptococcus pneumoniae polysaccharide-carrier protein conjugates and an SNE, wherein the SNE comprises sorbitan trioleate (SPAN-85), polysorbate-20 (PS-20) or polysorbate-80 (PS-80), squalene, and an optional cationic lipid.

The present invention further provides a pneumococcal conjugate composition comprising Streptococcus pneumoniae polysaccharide-carrier protein conjugates and an SNE, wherein the SNE comprises sorbitan trioleate (SPAN-85), polysorbate-20 (PS-20), squalene, and an optional cationic lipid.

The present invention further provides a pneumococcal conjugate composition comprising Streptococcus pneumoniae polysaccharide-carrier protein conjugates and an SNE, wherein the SNE comprises sorbitan trioleate (SPAN-85), polysorbate-20 (PS-20) or polysorbate-80 (PS-80), squalene, and a cationic lipid.

The present invention further provides a pneumococcal conjugate composition comprising Streptococcus pneumoniae polysaccharide-carrier protein conjugates and an SNE, wherein the SNE comprises sorbitan trioleate (SPAN-85), polysorbate-20 (PS-20), squalene, and a cationic lipid.

The present invention further provides a pneumococcal conjugate composition comprising Streptococcus pneumoniae polysaccharide-carrier protein conjugates and an SNE, wherein the SNE comprises sorbitan trioleate (SPAN-85), polysorbate-20 (PS-20) or polysorbate-80 (PS-80), squalene, and the cationic lipid (13Z,16Z)—N, N-dimethyl-3-nonyldocosa-13,16-dien-1-amine.

The present invention further provides a pneumococcal conjugate composition comprising Streptococcus pneumoniae polysaccharide-carrier protein conjugates and an SNE, wherein the SNE comprises sorbitan trioleate (SPAN-85), polysorbate-20 (PS-20), squalene, and the cationic lipid (13Z,16Z)—N, N-dimethyl-3-nonyldocosa-13,16-dien-1-amine.

The present invention further provides a pneumococcal conjugate vaccine comprising 1) Streptococcus pneumoniae polysaccharide-carrier protein conjugates and 2) an SNE comprising i) sorbitan trioleate (SPAN-85); (ii) polysorbate-20 (PS-20) or polysorbate-80 (PS-80); iii) squalene; and an optional iv) cationic lipid. In some embodiments, the pneumococcal conjugate vaccine does not contain a cationic lipid.

The present invention further provides a pneumococcal conjugate vaccine comprising 1) Streptococcus pneumoniae polysaccharide-carrier protein conjugates and 2) an SNE comprising i) sorbitan trioleate (SPAN-85); (ii) polysorbate-20 (PS-20); iii) squalene; and an optional iv) cationic lipid. In some embodiments, the pneumococcal conjugate vaccine does not contain a cationic lipid.

The present invention further provides a pneumococcal conjugate vaccine comprising 1) Streptococcus pneumoniae polysaccharide-carrier protein conjugates and 2) an SNE comprising i) sorbitan trioleate (SPAN-85); (ii) polysorbate-20 (PS-20) or polysorbate-80 (PS-80); iii) squalene; and iv) a cationic lipid.

The present invention further provides a pneumococcal conjugate vaccine comprising 1) Streptococcus pneumoniae polysaccharide-carrier protein conjugates and 2) an SNE comprising i) sorbitan trioleate (SPAN-85); (ii) polysorbate-20 (PS-20); iii) squalene; and iv) a cationic lipid.

The present invention further provides a pneumococcal conjugate vaccine comprising 1) Streptococcus pneumoniae polysaccharide-carrier protein conjugates and 2) an SNE comprising i) sorbitan trioleate (SPAN-85); (ii) polysorbate-20 (PS-20) or polysorbate-80 (PS-80); iii) squalene; and iv) (13Z,16Z)—N, N-dimethyl-3-nonyldocosa-13,16-dien-1-amine.

The present invention further provides a pneumococcal conjugate vaccine comprising 1) Streptococcus pneumoniae polysaccharide-carrier protein conjugates and 2) an SNE comprising i) sorbitan trioleate (SPAN-85); (ii) polysorbate-20 (PS-20); iii) squalene; and iv) (13Z,16Z)—N, N-dimethyl-3-nonyldocosa-13,16-dien-1-amine.

The present invention further provides a pneumococcal conjugate vaccine comprising 1) Streptococcus pneumoniae polysaccharide-carrier protein conjugates and 2) an SNE comprising i) sorbitan trioleate (SPAN-85); (ii) polysorbate-20 (PS-20) or polysorbate-80 (PS-80); iii) squalene; and iv) a cationic lipid, wherein the cationic lipid is not associated with a lipid nanoparticle (LNP).

The present invention further provides a pneumococcal conjugate vaccine comprising 1) Streptococcus pneumoniae polysaccharide-carrier protein conjugates and 2) an SNE comprising i) sorbitan trioleate (SPAN-85); (ii) polysorbate-20 (PS-20); iii) squalene; and iv) a cationic lipid, wherein the cationic lipid is not associated with a lipid nanoparticle (LNP).

The present invention further provides a pneumococcal conjugate composition, as described above, and a pharmaceutically acceptable carrier.

In an embodiment, the composition comprises the cationic lipid CLA, CLX, or CLY.

In an embodiment, the composition comprises the cationic lipid CLA.

In an embodiment, the composition comprises a cationic lipid selected from DLinDMA, DLinKC2DMA, DLin-MC3-DMA, CLinDMA, and S-Octyl CLinDMA.

In one embodiment, each of the Streptococcus pneumoniae polysaccharide-carrier protein conjugates in the composition comprises a polysaccharide of a particular Streptococcus pneumoniae serotype, wherein the polysaccharides in the conjugates comprise one or more serotypes selected from any known serotype, including, but not limited to serotypes 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20, 20A, 20B, 22F, 23A, 23B, 23F, 24F, 33F, 35B, 35F, and 38. In another embodiment, the serotypes comprise, consist essentially of, or consist of 4, 6B, 9V, 14, 18C, 19F and 23F. In another embodiment, the serotypes comprise, consist essentially of, or consist of 1, 3, 4, 5, 6A, 6B, 7F, 9V, 14, 18C, 19A, 19F and 23F. In another embodiment, the serotypes comprise, consist essentially of, or consist of 1, 3, 4, 5, 6A, 6B, 7F, 9V, 14, 18C, 19A, 19F, 22F, 23F and 33F. In another embodiment, the serotypes comprise, consist essentially of, or consist of 1, 3, 4, 5, 6A, 6B, 7F, 8, 9V, 10A, 11A, 12F, 14, 15B, 18C, 19A, 19F, 22F, 23F and 33F. In another embodiment, the serotypes comprise, consist essentially of, or consist of 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In another embodiment, the serotypes comprise, consist essentially of, or consist of 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B. In another embodiment, the serotypes comprise, consist essentially of, or consist of 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In another embodiment the serotypes comprise, consist essentially of, or consist of 3, 6A, 7F, 8, 9N, 10A, 11A, 12F, 15A, 15B, 16F, 17F, 19A, 20A, 22F, 23A, 23B, 24F, 31, 33F and 35B. In another embodiment the serotypes comprise, consist essentially of, or consist of 3, 6A, 7F, 8, 9N, 10A, 11A, 12F, 15A, de-O-acetylated-15B, 16F, 17F, 19A, 20A, 22F, 23A, 23B, 24F, 31, 33F and 35B. In another embodiment the serotypes comprise, consist essentially of, or consist of 3, 6A, 7F, 8, 9N, 10A, 11A, 12F, 15A, 15C, 16F, 17F, 19A, 20A, 22F, 23A, 23B, 24F, 31, 33F and 35B. In another embodiment the serotypes comprise, consist essentially of, or consist of 3, 6A, 7F, 8, 9N, 10A, 11A, 12F, 15A, 15B, 16F, 17F, 19A, 20, 22F, 23A, 23B, 24F, 31, 33F and 35B. In another embodiment the serotypes comprise, consist essentially of, or consist of 3, 6A, 7F, 8, 9N, 10A, 11A, 12F, 15A, de-O-acetylated-15B, 16F, 17F, 19A, 20, 22F, 23A, 23B, 24F, 31, 33F and 35B. In another embodiment the serotypes comprise, consist essentially of, or consist of 3, 6A, 7F, 8, 9N, 10A, 11A, 12F, 15A, 15C, 16F, 17F, 19A, 20, 22F, 23A, 23B, 24F, 31, 33F and 35B. In another embodiment the serotypes comprise, consist essentially of, or consist of 3, 6A, 7F, 8, 9N, 10A, 11A, 12F, 15A, 15B, 16F, 17F, 19A, 20B, 22F, 23A, 23B, 24F, 31, 33F and 35B. In another embodiment the serotypes comprise, consist essentially of, or consist of 3, 6A, 7F, 8, 9N, 10A, 11A, 12F, 15A, de-O-acetylated-15B, 16F, 17F, 19A, 20B, 22F, 23A, 23B, 24F, 31, 33F and 35B. In another embodiment the serotypes comprise, consist essentially of, or consist of 3, 6A, 7F, 8, 9N, 10A, 11A, 12F, 15A, 15C, 16F, 17F, 19A, 20B, 22F, 23A, 23B, 24F, 31, 33F and 35B.

In one embodiment, the polysaccharide-carrier protein conjugates comprise polysaccharides that are selected from a group of pneumococcal serotypes which consist of serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38. In another embodiment, the group of serotypes consists of 4, 6B, 9V, 14, 18C, 19F and 23F. In another embodiment, the group of serotypes consists of 1, 3, 4, 5, 6A, 6B, 7F, 9V, 14, 18C, 19A, 19F and 23F. In another embodiment, the group of serotypes consists of 1, 3, 4, 5, 6A, 6B, 7F, 9V, 14, 18C, 19A, 19F, 22F, 23F and 33F. In another embodiment, the group of serotypes consists of 1, 3, 4, 5, 6A, 6B, 7F, 8, 9V, 10A, 11A, 12F, 14, 15B, 18C, 19A, 19F, 22F, 23F and 33F. In another embodiment, the group of serotypes consists of 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In another embodiment, the group of serotypes consists of 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B. In another embodiment, the group of serotypes consists of 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In another embodiment the group of serotypes consists of 3, 6A, 7F, 8, 9N, 10A, 11A, 12F, 15A, 15B, 16F, 17F, 19A, 20A, 22F, 23A, 23B, 24F, 31, 33F and 35B. In another embodiment the group of serotypes consists of 3, 6A, 7F, 8, 9N, 10A, 11A, 12F, 15A, de-O-acetylated-15B, 16F, 17F, 19A, 20A, 22F, 23A, 23B, 24F, 31, 33F and 35B. In another embodiment the group of serotypes consists of 3, 6A, 7F, 8, 9N, 10A, 11A, 12F, 15A, 15C, 16F, 17F, 19A, 20A, 22F, 23A, 23B, 24F, 31, 33F and 35B. In another embodiment the group of serotypes consists of 3, 6A, 7F, 8, 9N, 10A, 11A, 12F, 15A, 15B, 16F, 17F, 19A, 20, 22F, 23A, 23B, 24F, 31, 33F and 35B. In another embodiment the group of serotypes consists of 3, 6A, 7F, 8, 9N, 10A, 11A, 12F, 15A, de-O-acetylated-15B, 16F, 17F, 19A, 20, 22F, 23A, 23B, 24F, 31, 33F and 35B. In another embodiment the group of serotypes consists of 3, 6A, 7F, 8, 9N, 10A, 11A, 12F, 15A, 15C, 16F, 17F, 19A, 20, 22F, 23A, 23B, 24F, 31, 33F and 35B. In another embodiment the group of serotypes consists of 3, 6A, 7F, 8, 9N, 10A, 11A, 12F, 15A, 15B, 16F, 17F, 19A, 20B, 22F, 23A, 23B, 24F, 31, 33F and 35B. In another embodiment the group of serotypes consists of 3, 6A, 7F, 8, 9N, 10A, 11A, 12F, 15A, de-O-acetylated-15B, 16F, 17F, 19A, 20B, 22F, 23A, 23B, 24F, 31, 33F and 35B. In another embodiment the group of serotypes consists of 3, 6A, 7F, 8, 9N, 10A, 11A, 12F, 15A, 15C, 16F, 17F, 19A, 20B, 22F, 23A, 23B, 24F, 31, 33F and 35B.

In an embodiment, the present invention provides a pneumococcal conjugate composition comprising 7 distinct Streptococcus pneumoniae polysaccharide-carrier protein conjugates and any of the stable nanoemulsions (SNE) described herein, wherein the Streptococcus pneumoniae polysaccharides consist of serotypes 4, 6B, 9V, 14, 18C, 19F and 23F.

In an embodiment, the present invention provides a pneumococcal conjugate composition comprising 13 distinct Streptococcus pneumoniae polysaccharide-carrier protein conjugates and an SNE as described herein, wherein the Streptococcus pneumoniae polysaccharides consist of serotypes 1, 3, 4, 5, 6A, 6B, 7F, 9V, 14, 18C, 19A, 19F and 23F.

In an embodiment, the present invention provides a pneumococcal conjugate composition comprising 15 distinct Streptococcus pneumoniae polysaccharide-carrier protein conjugates and an SNE as described herein, wherein the Streptococcus pneumoniae polysaccharides consist of serotypes 1, 3, 4, 5, 6A, 6B, 7F, 9V, 14, 18C, 19A, 19F, 22F, 23F and 33F.

In an embodiment, the present invention provides a pneumococcal conjugate composition comprising 20 distinct Streptococcus pneumoniae polysaccharide-carrier protein conjugates and an SNE as described herein, wherein the Streptococcus pneumoniae polysaccharides consist of serotypes 1, 3, 4, 5, 6A, 6B, 7F, 8, 9V, 10A, 11A, 12F, 14, 15B, 18C, 19A, 19F, 22F, 23F and 33F.

In an embodiment, the present invention provides a pneumococcal conjugate composition comprising 24 distinct Streptococcus pneumoniae polysaccharide-carrier protein conjugates and an SNE as described herein, wherein the Streptococcus pneumoniae polysaccharides consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B.

In an embodiment, the present invention provides a pneumococcal conjugate composition comprising 24 distinct Streptococcus pneumoniae polysaccharide-carrier protein conjugates and an SNE as described herein, wherein the Streptococcus pneumoniae polysaccharides consist of serotypes 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B.

In an embodiment, the present invention provides a pneumococcal conjugate composition comprising 24 distinct Streptococcus pneumoniae polysaccharide-carrier protein conjugates and an SNE as described herein, wherein the Streptococcus pneumoniae polysaccharides consist of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B.

In an embodiment, the present invention provides a pneumococcal conjugate composition comprising 21 distinct Streptococcus pneumoniae polysaccharide-carrier protein conjugates and an SNE as described herein, wherein the Streptococcus pneumoniae polysaccharides consist of serotypes 3, 6A, 7F, 8, 9N, 10A, 11A, 12F, 15A, 15B, 16F, 17F, 19A, 20, 22F, 23A, 23B, 24F, 31, 33F and 35B.

In an embodiment, the present invention provides a pneumococcal conjugate composition comprising 21 distinct Streptococcus pneumoniae polysaccharide-carrier protein conjugates and an SNE as described herein, wherein the Streptococcus pneumoniae polysaccharides consist of serotypes 3, 6A, 7F, 8, 9N, 10A, 11A, 12F, 15A, de-O-acetylated-15B, 16F, 17F, 19A, 20, 22F, 23A, 23B, 24F, 31, 33F and 35B.

In an embodiment, the present invention provides a pneumococcal conjugate composition comprising 21 distinct Streptococcus pneumoniae polysaccharide-carrier protein conjugates and an SNE as described herein, wherein the Streptococcus pneumoniae polysaccharides consist of serotypes 3, 6A, 7F, 8, 9N, 10A, 11A, 12F, 15A, 15C, 16F, 17F, 19A, 20, 22F, 23A, 23B, 24F, 31, 33F and 35B.

In an embodiment, the present invention provides a pneumococcal conjugate composition comprising 7 distinct Streptococcus pneumoniae polysaccharide-carrier protein conjugates, wherein the Streptococcus pneumoniae polysaccharides consist of serotypes 4, 6B, 9V, 14, 18C, 19F and 23F, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 13 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F and 23F, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 15 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 20 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15B, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, de-O-acetylated-15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15C, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 7 distinct Streptococcus pneumoniae polysaccharide-carrier protein conjugates, wherein the Streptococcus pneumoniae polysaccharides consist of serotypes 4, 6B, 9V, 14, 18C, 19F and 23F, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 13 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F and 23F, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 15 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 20 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15B, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene and a cationic lipid.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, de-O-acetylated-15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15C, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene and a cationic lipid.

In an embodiment, the present invention provides a pneumococcal conjugate composition comprising 7 distinct Streptococcus pneumoniae polysaccharide-carrier protein conjugates, wherein the Streptococcus pneumoniae polysaccharides consist of serotypes 4, 6B, 9V, 14, 18C, 19F and 23F, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 13 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F and 23F, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 15 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 20 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15B, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene and the cationic lipid CLA.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, de-O-acetylated-15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15C, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene and the cationic lipid CLA.

In an embodiment of the compositions above, the SNE comprises PS-20. In another embodiment of the compositions above, the SNE comprises PS-80 In an embodiment of the compositions above, the compositions do not comprise polysaccharide-carrier protein conjugates containing polysaccharides of any other Streptococcus pneumoniae serotype.

In an embodiment of the compositions of the invention, the carrier protein is selected from OMPC, PhtD, pLys, DT (Diphtheria toxoid), TT (tetanus toxoid), fragment C of TT, pertussis toxoid, cholera toxoid and CRM197. In another embodiment, the carrier protein is CRM197.

The present invention provides the compositions above further comprising 5 mM-40 mM histidine at pH 5.1-7.0 and 25 mM-300 mM NaCl.

The present invention provides the compositions above further comprising about 20 mM histidine at about pH 5.8 and about 75 mM NaCl.

The present invention provides the compositions above further comprising 20 mM histidine at pH 5.8 and 75 mM NaCl.

The present invention provides the compositions above further comprising 5 mM-40 mM histidine at pH 5.1-7.0, 0.0125%-0.2% PS-20 or PS-80, and 25 mM-300 mM NaCl.

The present invention provides the compositions above further comprising about 20 mM histidine at about pH 5.8, 0.05% PS-20 or PS-80, and about 75 mM NaCl.

The present invention provides the compositions above further comprising 20 mM histidine at pH 5.8, 0.05% PS-20 or PS-80, and 75 mM NaCl.

The present invention also provides a method of making a pneumococcal conjugate composition comprising 1) Streptococcus pneumoniae polysaccharide-carrier protein conjugates and 2) a SNE comprising i) sorbitan trioleate (SPAN-85); (ii) polysorbate-20 (PS-20) or polysorbate-80 (PS-80); iii) squalene; and an optional iv) cationic lipid.

The present invention also provides a method of making a pneumococcal conjugate composition comprising 1) Streptococcus pneumoniae polysaccharide-carrier protein conjugates and 2) a SNE comprising i) sorbitan trioleate (SPAN-85); (ii) polysorbate-20 (PS-20); iii) squalene; and an optional iv) cationic lipid.

The present invention also provides a method of making a pneumococcal conjugate composition comprising Streptococcus pneumoniae polysaccharide-carrier protein conjugates and an SNE.

The present invention also provides methods of treatment or prevention of pneumococcal diseases with a pneumococcal conjugate composition of the instant invention.

The present invention also provides a use of the pneumococcal conjugate composition of the instant invention for the treatment or prevention of pneumococcal diseases.

### Definitions

As used throughout the specification and in the appended claims, the singular forms “a,” “an,” and “the” include the plural reference unless the context clearly dictates otherwise.

As used throughout the specification and appended claims, the following abbreviations and definitions apply:
APA aluminum phosphate adjuvant
CLA ((13Z,16Z)—N,N-dimethyl-3-nonyldocosa-13,16-dien-1-amine)
DMSO dimethylsulfoxide
ECL electrochemiluminescence
GMT geometric mean titer
HPSEC high performance size exclusion chromatography
ID intradermal
IM intramuscular
LNP lipid nanoparticle
LOS lipooligosaccharide
LPS lipopolysaccharide
ME microemulsion
MNS microfluidic nanoemulsion self-assembly
Mw molecular weight
NE nanoemulsion
NMWCO nominal molecular weight cut off
OPA opsonophagocytic assay
PCV pneumococcal conjugate vaccine
PD1 post dose 1
PD2 post dose 2
PD3 post dose 3
PHE pre-homogenized emulsion
PnPs pneumococcal polysaccharide
Ps polysaccharide
PS-20 polysorbate-20
PS-80 polysorbate-80
SNE stable nanoemulsion
SPAN-85 sorbitan-trioleate
ST6B or ST-6B serotype 6B
w/v weight per volume

As used herein, the term “about,” when used herein in reference to a value, refers to a value that is the same as or, in context, is similar to the referenced value. In general, those skilled in the art, familiar with the context, will appreciate the absolute amount and/or relative degree of difference encompassed by “about” in that context. For example, in some embodiments, the term “about” can encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

As used herein, the term “alkenyl” refers to a straight chain, cyclic or branched unsaturated aliphatic hydrocarbon having the specified number of carbon atoms. In one embodiment, an alkenyl group contains from 8 to 24 carbon atoms (C₈-C₂₄ alkenyl). In one embodiment, an alkenyl group is linear. In another embodiment, an alkenyl group is branched. In another embodiment the alkenyl group is unsubstituted.

As used herein, the term “alkyl” refers to a straight chain, cyclic or branched saturated aliphatic hydrocarbon having the specified number of carbon atoms. In one embodiment, an alkyl group contains from 8 to 24 carbon atoms (C₈-C₂₄ alkyl). In one embodiment, an alkyl group is linear. In another embodiment, an alkyl group is branched. In another embodiment the alkyl group is unsubstituted.

An “adjuvant,” as defined herein, is a substance that serves to enhance (e.g., increase, accelerate, prolong or modulate) the immunogenicity of a composition of the invention. As disclosed herein, an SNE is to be used as the adjuvant in accordance with the instant invention. An adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. As used herein, an “adjuvanted composition” is a composition that comprises an adjuvant.

As used herein, the term “administration” refers to the act of providing an active agent, composition, or formulation to a subject. Exemplary routes of administration to the human body can be through the eyes (ophthalmic), mouth (oral), skin (transdermal), nose (nasal), lungs (inhalant), rectal, vaginal, oral mucosa (buccal), ear, by injection (e.g., intravenously (IV), subcutaneously, intratumorally, intraperitoneally, intramuscular (IM), intradermal (ID) etc.) and the like.

The term “antigen” refers to any antigen that can generate one or more immune responses. The antigen may be a protein (including recombinant proteins), polypeptide, or peptide (including synthetic peptides). In certain embodiments, the antigen is a lipid or a carbohydrate (polysaccharide). In certain embodiments, the antigen is a protein extract, cell (including tumor cell), or tissue. The antigen may be one that generates a humoral and/or CTL immune response. Antigens of the instant invention are Streptococcus pneumoniae polysaccharides.

The term “cationic lipid” refers to a lipid species that carries a net positive charge at a selected pH, such as physiological pH. A cationic lipid may be utilized as an ingredient in a multi-component SNE adjuvant formulation. Those of skill in the art will appreciate that a cationic lipids can include, but are not limited to, those disclosed in US Patent Application Publication Nos. US2008/0085870, US2008/0057080, US2009/0263407, US2009/0285881, US2010/0055168, US2010/0055169, US2010/0063135, US2010/0076055, US2010/0099738, US2010/0104629, US2013/0017239, and US2016/0361411, International Application Publication Nos. WO2011/022460, WO2012/040184, WO2011/076807, WO2010/021865, WO 2009/132131, WO2010/042877, WO2010/146740, and WO2010/105209, and U.S. Pat. Nos. 5,208,036, 5,264,618, 5,279,833, 5,283,185, 6,890,557, and 9,669,097.

As used herein, the term “composition” refers to a formulation containing an active pharmaceutical or biological ingredient (for example, a pneumococcal polysaccharide-carrier protein conjugate and a SNE), along with one or more additional components. The term “composition” is used interchangeably with “pharmaceutical composition” and “formulation”. The compositions can be liquid or solid (e.g. lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. Compositions are nontoxic to recipients at the dosages and concentrations employed.

The term “comprises” when used with the composition of the invention refers to the inclusion of any other components, such as adjuvants and excipients, or the addition of one or more polysaccharide-carrier protein conjugates that are not specifically enumerated.

The term “consisting of” or “consists of” when used with the multivalent polysaccharide-carrier protein conjugate formulation(s) refers to a formulation having those particular Streptococcus pneumoniae polysaccharide-carrier protein conjugates and no other Streptococcus pneumoniae polysaccharide-carrier protein conjugates from a different serotype.

“Consists essentially of” and variations such as “consist essentially of” or “consisting essentially of,” indicate the inclusion of any recited elements or group of elements, and the optional inclusion of other elements, of similar or different nature than the recited elements, which do not materially change the basic or novel properties of the specified dosage regimen, method, or composition.

As used herein, the term “de-O-acetylated-15B” or “de-O-acetyl-15B” or “de-O-Ac-15B” refers to a de-O-acetylated serotype 15B wherein the O-Acetyl content is less than 5% per repeating unit. In another embodiment the O-Acetyl content is less than 1% per repeating unit. In another embodiment the O-Acetyl content is less than 0.5% per repeating unit. In another embodiment the O-Acetyl content is less than 0.1% per repeating unit. Processes for de-O-acetylation are known in the art, for example as described in Rajam et al., Clinical and Vaccine Immunology, 2007, 14(9):1223-1227.

As used herein, the term “dose” means a quantity of an agent, API (active pharmaceutical ingredient), formulation, or composition taken or recommended to be taken at a particular time.

As used herein, the term “immunogenic” or “immunogenicity” refers to the ability of an antigen to provoke an immune response in a subject. The term “immunogenic composition” refers to the ability of an agent, API, formulation, or composition to provoke an immune response in a subject. The pneumococcal conjugate compositions of the instant invention are immunogenic compositions.

Those “in need of treatment” include those previously exposed to or infected with Streptococcus pneumoniae, those who were previously vaccinated against Streptococcus pneumoniae, as well as those prone to have an infection or any person in which a reduction in the likelihood of infection is desired, e.g., the immunocompromised, the elderly, children, adults, or healthy individuals.

The phrase “indicated for the prevention of pneumococcal disease” means that a vaccine or composition is approved by one or more regulatory authorities, such as the US Food and Drug Administration, for the prophylaxis of one or more diseases caused by any serotype of Streptococcus pneumoniae, including, but not limited to: pneumococcal disease generally, pneumococcal pneumonia, pneumococcal meningitis, pneumococcal bacteremia, invasive disease caused by Streptococcus pneumoniae, and otitis media caused by Streptococcus pneumoniae.

As used herein, the term “lipid” refers to any of a group of organic compounds that are esters of fatty acids and are characterized by being insoluble in water or having low solubility in water but may be soluble in many organic solvents. Lipids can be divided in at least three classes: (1) “simple lipids,” which include, e.g., fats and oils as well as waxes; (2) “compound lipids,” which include, e.g., phospholipids and glycolipids; and (3) “derived lipids,” which include, e.g., steroids.

As used herein, the term “lipid nanoparticle” (or “LNP”) refers to a lipid composition that forms a particle having a length or width measurement (e.g., a maximum length or width measurement) between 10 and 1000 nanometers and comprises more than one class and/or type of lipid. For example, according to the instant invention, a lipid nanoparticle cannot be solely comprised of cationic lipids.

As used herein, the term “neutral lipid” refers to a lipid species that exists either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides and diacylglycerols.

A “patient” (alternatively referred to herein as a “subject”) refers to a mammal capable of being infected with Streptococcus pneumoniae. In preferred embodiments, the patient is a human. A patient can be treated prophylactically or therapeutically. Prophylactic treatment provides sufficient protective immunity to reduce the likelihood or severity of a pneumococcal infection or the effects thereof, e.g., pneumococcal pneumonia. Therapeutic treatment can be performed to reduce the severity or prevent recurrence of a Streptococcus pneumoniae infection or the clinical effects thereof. Prophylactic treatment can be performed using a pneumococcal conjugate composition or vaccine of the invention, as described herein. The pneumococcal conjugate compositions or vaccines of the invention can be administered to the general population or to those persons at an increased risk of pneumococcal infection, e.g. the elderly, or those who live with or care for the elderly.

“PCV1”, as used herein, refers to a 1-valent pneumococcal conjugate vaccine or composition comprising one Streptococcus pneumoniae polysaccharide-carrier protein conjugate, comprising capsular polysaccharide from a Streptococcus pneumoniae serotype conjugated to a carrier protein. In specific embodiments, the carrier protein is CRM197.

“PCV13”, as used herein, refers to a 13-valent pneumococcal conjugate vaccine or composition comprising thirteen Streptococcus pneumoniae polysaccharide-carrier protein conjugates, each comprising capsular polysaccharide from a Streptococcus pneumoniae serotype conjugated to a carrier protein, wherein the serotypes of Streptococcus pneumoniae are: 1, 3, 4, 5, 6A, 6B, 7F, 9V, 14, 18C, 19A, 19F, and 23F In specific embodiments, the carrier protein of each of Streptococcus pneumoniae polysaccharide-carrier protein conjugates is CRM197.

“PCV21”, as used herein, refers to a 21-valent pneumococcal conjugate vaccine or composition comprising twenty Streptococcus pneumoniae polysaccharide-carrier protein conjugates, each comprising capsular polysaccharide from a Streptococcus pneumoniae serotype conjugated to a carrier protein, wherein the serotypes of Streptococcus pneumoniae are: 3, 6A, 7F, 8, 9N, 10A, 11A, 12F, 15A, 16F, 17F, 19A, 20, 22F, 23A, 23B, 24F, 31, 33F and 35B, and one of the following serogroup 15 serotypes: 15B, 15C or de-O-acetylated-15B. In a particular embodiment, the serogroup 15 serotype is serotype 15C or de-O-acetylated-15B. In another embodiment, the serogroup 15 serotype is serotype de-O-acetylated-15B. In specific embodiments, the carrier protein of one or more of the Streptococcus pneumoniae polysaccharide-carrier protein conjugates is CRM197. In further embodiments, the carrier protein of each of the Streptococcus pneumoniae polysaccharide-carrier protein conjugates is CRM197.

“PCV24”, as used herein, refers to a 24-valent pneumococcal conjugate vaccine or composition comprising twenty-three Streptococcus pneumoniae polysaccharide-carrier protein conjugates, each comprising capsular polysaccharide from a Streptococcus pneumoniae serotype conjugated to a carrier protein, wherein the serotypes of Streptococcus pneumoniae are: 1, 3, 4, 5, 6A, 6B, 7F, 8, 9V, 10A, 11A, 12F, 14, 15A, 18C, 19A, 19F, 22F, 23B, 23F, 24F, 33F and 35B, and one of the following serogroup 15 serotypes: 15B, 15C or de-O-acetylated-15B. In a particular embodiment, the serogroup 15 serotype is serotype 15C or de-O-acetylated-15B. In another embodiment, the serogroup 15 serotype is serotype de-O-acetylated-15B. In specific embodiments, the carrier protein of one or more of the Streptococcus pneumoniae polysaccharide-carrier protein conjugates is CRM197. In further embodiments, the carrier protein of each of the Streptococcus pneumoniae polysaccharide-carrier protein conjugates is CRM197.

With respect to a carrier, diluent, or excipient of a pharmaceutical composition, the term “pharmaceutically acceptable” indicates that a carrier, diluent, or excipient must be compatible with the other ingredients of the composition and not deleterious to the recipient thereof.

As used herein, the terms “pneumococcal conjugate” or “pneumococcal polysaccharide-carrier protein conjugate” refer to a Streptococcus pneumoniae polysaccharide-carrier protein conjugate.

As used herein, the term “pneumococcal conjugate vaccine” (or “PCV”) is a pharmaceutical preparation or composition comprising pneumococcal polysaccharide-carrier protein conjugate(s) that provide active immunity to disease or pathological conditions caused by serotype(s) of Streptococcus pneumoniae.

As used herein, the terms “stable nanoemulsion” or “SNE” refer to a formulation of emulsifiers and/or solubilizers and/or surfactants and/or lipids that have adjuvant properties in a pneumococcal conjugate vaccine. Specifically, SNE refers to a SNE adjuvant formulation comprising 1) sorbitan trioleate (SPAN-85); 2) polysorbate-20 (PS-20); 3) squalene; and an optional 4) cationic lipid.

“Surfactants” are stabilizing ingredients in a multi-component SNE adjuvant formulation and include the polyoxyethylene sorbitan esters surfactants (commonly referred to as the Tweens, especially PS-20 and PS-80), copolymers of ethylene oxide (EO), propylene oxide (PO), and/or butylene oxide (BO), sold under the DOWFAX™ tradename, such as linear EO/PO block copolymers (poloxamers); octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span-85, Tween-85 or [2[(2R,3S,41?)-4-hydroxy-3-[(Z)-octadec-9-enoyl]oxyoxolan-2-yl]-2-[(Z)-octadec-9-enoyl]oxyethyl] (Z)-octadec-9-enoate) and sorbitan monolaurate. In an embodiment, surfactants are sorbitan esters and poloxamers. In an embodiment, surfactants are polysorbate-20 (PS-20) and polysorbate-80 (PS-80).

“Terpenes” are stabilizing ingredients in a multi-component SNE adjuvant formulation and include, but are not limited to: monoterpenes including geraniol, terpineol, limonene, myrcene, linalool and pinene; sesquiterpenes including humulene, farnesenes and farnesol; diterpenes including cafestol, kahweol, cembrene and taxadiene; triterpenes including squalene and squalante; tetraterpenes including acyclic lycopene, monocyclic gamma-carotene, bicyclic alpha- and beta-carotenes; polyterpines and norisopredoids. In an embodiment, a terpene is squalene.

The term “therapeutically effective amount” refers to an amount of the composition or vaccine sufficient to produce the desired therapeutic effect in a human or animal, e.g. the amount necessary to elicit an immune response, treat, cure, prevent, or inhibit development and progression of a disease or the symptoms thereof and/or the amount necessary to ameliorate symptoms or cause regression of a disease. One of skill in the art can readily determine a therapeutically effective amount of a given composition or vaccine.

As used herein, the term “valent” refers to the presence of a specified number of polysaccharide-carrier protein conjugates in a composition.

As used herein, the term “vaccine” or “vaccine composition” refers to a biological preparation used to stimulate the production of antibodies and provide immunity against an infectious disease.

### Cationic Lipids

Cationic lipids and methods of making cationic lipids are well known in the art.

In some embodiments, the cationic lipid includes any cationic lipid mentioned in U.S. Patent Application Publication Nos. US 2008/0085870, US 2008/0057080, US 2009/0263407, US 2009/0285881, US 2010/0055168, US 2010/0055169, US 2010/0063135, US 2010/0076055, US 2010/0099738, US 2010/0104629, US 2013/0017239, and US 2016/0361411, International Patent Application Publication No. WO2011/022460 A1; WO2012/040184, WO2011/076807, WO2010/021865, WO 2009/132131, WO2010/042877, WO2010/146740, WO2010/105209, and in U.S. Pat. Nos. 5,208,036, 5,264,618, 5,279,833, 5,283,185, 6,890,557, and 9,669,097.

In some embodiments, cationic lipids of the instant invention have the following structure, illustrated by Formula 1:

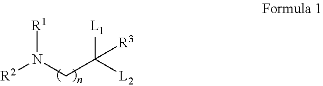

wherein:

R¹ and R² are each methyl;

R³ is H;

n is 1 or 2;

L₁ is selected from C₈-C₂₄ alkyl and C₈-C₂₄ alkenyl; and

L₂ is selected from C₄-C₉ alkyl and C₄-C₉ alkenyl;

or any pharmaceutically acceptable salt or stereoisomer thereof.

A cationic lipid may be an aminoalkyl lipid.

A cationic lipid may be an asymmetric aminoalkyl lipid.

In an embodiment of the instant invention, the cationic lipid is (13Z,16Z)—N,N-dimethyl-3-nonyldocosa-13,16-dien-1-amine (CLA); or (6Z,9Z,26Z,29Z)—N,N-dimethylpentatriaconta-6,9,26,29-tetraen-18-amine (CLX); or N,N-dimethyl-1-((1S,2R)-2-octylcyclopropyl)heptadecan-8-amine (CLY).

In another embodiment of the instant invention, the cationic lipid is selected from:
DLinDMA;
DLinKC2DMA;
DLin-MC3-DMA;
CLinDMA;
S-Octyl CLinDMA;
(2S)-1-{7-[(3P)-cholest-5-en-3-yloxy]heptyloxy}-3-[(4Z)-dec-4-en-1-yloxy]-N,N-dimethylpropan-2-amine;
(2R)-1-{4-[(3P)-cholest-5-en-3-yloxy]butoxy}-3-[(4Z)-dec-4-en-1-yloxy]-N,N-dimethylpropan-2-amine;
1-[(2R)-1-{4-[(3β)-cholest-5-en-3-yloxy]butoxy}-3-(octyloxy)propan-2-yl]guanidine;
1-[(2R)-1-{7-[(3β)-cholest-5-en-3-yloxy]heptyloxy}-N,N-dimethyl-3-[(9Z,12Z)-octadeca-9,12-dien-1-yloxy]propan-2-amine;
1-[(2R)-1-{4-[(3β)-cholest-5-en-3-yloxy]butoxy}-N,N-dimethyl-3-[(9Z,12Z)-octadeca-9,12-dien-1-yloxy]propan-2-amine;
(2S)-1-({6-[(3P))-cholest-5-en-3-yloxy]hexyl}oxy)-N,N-dimethyl-3-[(9Z)-octadec-9-en-1-yloxy]propan-2-amine;
(3β)-3-[6-{[(2S)-3-[(9Z)-octadec-9-en-1-yloxyl]-2-(pyrrolidin-1-yl)propyl]oxy}hexyl)oxy]cholest-5-ene;
(2R)-1-{4-[(3P)-cholest-5-en-3-yloxy]butoxy}-3-(octyloxy)propan-2-amine;
(2R)-1-({8-[(3P)-cholest-5-en-3-yloxy]octyl}oxy)-N,N-dimethyl-3-(pentyloxy)propan-2-amine;
(2R)-1-({8-[(3P)-cholest-5-en-3-yloxy]octyl}oxy)-3-(heptyloxy)-N,N-dimethylpropan-2-amine;
(2R)-1-({8-[(3P)-cholest-5-en-3-yloxy]octyl}oxy)-N,N-dimethyl-3-[(2Z)-pent-2-en-1-yloxy]propan-2-amine;
(2S)-1-butoxy-3-({8-[(3P)-cholest-5-en-3-yloxy]octyl}oxy)-N,N-dimethylpropan-2-amine;
(2S-1-({8-[(3P)-cholest-5-en-3-yloxy]octyl}oxy)-3-[2,2,3,3,4,4,5,5,6,6,7,7,8,8,9,9-hexadecafluorononyl)oxy]-N,N-dimethylpropan-2-amine;
2-amino-2-{[(9Z, 12Z)-octadeca-9,12-dien-1-yloxy]methyl} propane-1,3-diol;
2-amino-3-({9-[(3β,8ξ,9ξ,14ξ,17ξ,20ξ)-cholest-5-en-3-yloxy]nonyl}oxy)-2-{[(9Z,12Z)-octadeca-9,12-dien-1-yloxy]methyl}propan-1-ol;
2-amino-3-({6-[(3β,8ξ,9ξ,14ξ,17ξ,20ξ)-cholest-5-en-3-yloxy]nonyl}oxy)-2-{[(9Z)-octadec-9-en-1-yloxy]methyl}propan-1-ol;
(20Z,23Z)—N,N-dimethylnonacosa-20,23-dien-10-amine;
(17Z,20Z)—N,N-dimethylhexacosa-17,20-dien-9-amine;
(16Z,19Z)—N,N-dimethylpentacosa-16,19-dien-8-amine;
(13Z,16Z)—N,N-dimethyldocosa-13,16-dien-5-amine;
(12Z,15Z)—N,N-dimethylhenicosa-12,15-dien-4-amine;
(14Z,17Z)—N,N-dimethyltricosa-14,17-dien-6-amine;
(15Z,18Z)—N,N-dimethyltetracosa-15,18-dien-7-amine;
(18Z,21 Z)—N,N-dimethylheptacosa-18,21-dien-10-amine;
(15Z,18Z)—N,N-dimethyltetracosa-15,18-dien-5-amine;
(14Z,17Z)—N,N-dimethyltricosa-14,17-dien-4-amine;
(19Z,22Z)—N,N-dimethyloctacosa-19,22-dien-9-amine;
(18Z,21Z)—N,N-dimethylheptacosa-18,21-dien-8-amine;
(17Z,20Z)—N,N-dimethylhexacosa-17,20-dien-7-amine;
(16Z,19Z)—N,N-dimethylpentacosa-16,19-dien-6-amine;
(22Z,25Z)—N,N-dimethylhentriaconta-22,25-dien-10-amine;
(21 Z,24Z)—N,N-dimethyltriaconta-21,24-dien-9-amine;
(18Z)—N,N-dimethylheptacos-18-en-10-amine;
(17Z)—N,N-dimethylhexacos-17-en-9-amine;
(19Z,22Z)—N,N-dimethyloctacosa-19,22-dien-7-amine;
N,N-dimethylheptacosan-10-amine;
(20Z,23Z)—N-ethyl-N-methylnonacosa-20,23-dien-10-amine;
1-[(11Z,14Z)-1-nonylicosa-11,14-dien-1-yl]pyrrolidine;
(20Z)—N,N-dimethylheptacos-20-en-10-amine;
(15Z)—N,N-dimethylheptacos-15-en-10-amine;
(14Z)—N,N-dimethylnonacos-14-en-10-amine;
(17Z)—N,N-dimethylnonacos-17-en-10-amine;
(24Z)—N,N-dimethyltritriacont-24-en-10-amine;
(20Z)—N,N-dimethylnonacos-20-en-10-amine;
(22Z)—N,N-dimethylhentriacont-22-en-10-amine;
(16Z)—N,N-dimethylpentacos-16-en-8-amine;
(12Z,15Z)—N,N-dimethyl-2-nonylhenicosa-12,15-dien-1-amine;
(13Z,16Z)—N,N-dimethyl-3-nonyldocosa-13,16-dien-1-amine;
N,N-dimethyl-1-[(1S,2R)-2-octylcyclopropyl]heptadecan-8-amine;
1-[(1 S,2R)-2-hexylcyclopropyl]-N,N-dimethylnonadecan-10-amine;
N,N-dimethyl-1-[(1S,2R)-2-octylcyclopropyl]nonadecan-10-amine;
N,N-dimethyl-21-[(1S,2R)-2-octylcyclopropyl]henicosan-10-amine;
N,N-dimethyl-1-[(1S,2S)-2-{[(1R,2R)-2-pentylcyclopropyl]methyl}cyclopropyl]nonadecan-10-amine;
N,N-dimethyl-1-[(1S,2R)-2-octylcyclopropyl]hexadecan-8-amine;
N,N-dimethyl-1-[(1R,2S)-2-undecylcyclopropyl]tetradecan-5-amine;
N,N-dimethyl-3-{7-[(1 S,2R)-2-octylcyclopropyl]heptyl}dodecan-1-amine;
1-[(1R,2S)-2-heptylcyclopropyl]-N,N-dimethyloctadecan-9-amine;
1-[(1S,2R)-2-decylcyclopropyl]-N,N-dimethylpentadecan-6-amine;
N,N-dimethyl-1-[(1S,2R)-2-octylcyclopropyl]pentadecan-8-amine; and
(11E,20Z,23Z)—N,N-dimethylnonacosa-11,20,23-trien-10-amine;
   or a pharmaceutically acceptable salt thereof, or a stereoisomer of any of the foregoing.

In another embodiment of the instant invention, the cationic lipid is selected from (13Z,16Z)—N,N-dimethyl-3-nonyldocosa-13,16-dien-1-amine, or a pharmaceutically acceptable salt or stereoisomer thereof.

In another embodiment of the instant invention, the cationic lipid is (13Z,16Z)—N,N-dimethyl-3-nonyldocosa-13,16-dien-1-amine (CLA).

The present disclosure provides, among other things, a composition that comprises pneumococcal conjugates and 3 SNE components 1) sorbitan trioleate (SPAN-85); 2) polysorbate-20 (PS-20) or polysorbate-80 (PS-80), and 3) squalene.

In some embodiments, the SNE comprises 32-97 mole % squalene, 1-34 mole % SPAN-85 and 1-34 mole % of PS-20 or PS-80.

In some embodiments, the SNE comprises 86-98 mole % squalene, 1-7 mole % SPAN-85 and 1-7 mole % of PS-20 or PS-80.

In some embodiments, the SNE comprises 92-94 mole % squalene, 3-4 mole % SPAN-85 and 3-4 mole % of PS-20 or PS-80.

In one embodiment of the invention, the SNE comprises 92.91 mole % squalene, 3.98 mole % SPAN-85 and 3.11 mole % of PS-20 or PS-80.

The present disclosure provides, among other things, a composition that comprises pneumococcal conjugates and 4 SNE components 1) a cationic lipid; 2) sorbitan trioleate (SPAN-85); 3) polysorbate-20 (PS-20) or polysorbate-80 (PS-80), and 4) squalene. A particular SNE composition comprises the cationic lipid (13Z,16Z)—N, N-dimethyl-3-nonyldocosa-13,16-dien-1-amine (“CLA” or, when the cationic lipid is included in the SNE, “CLA-SNE”).

In some embodiments, the SNE comprises 1-60 mole % cationic lipid, 32-97 mole % squalene, 1-4 mole % SPAN-85 and 1-4 mole % of PS-20 or PS-80.

In some embodiments, the SNE comprises 10-14 mole % cationic lipid, 78-84 mole % squalene, 1-6 mole % SPAN-85 and 1-6 mole % of PS-20 or PS-80.

In some embodiments, the SNE comprises 40-46 mole % cationic lipid, 41-52 mole % squalene, 1-8 mole % SPAN-85 and 1-8 mole % of PS-20 or PS-80.

In one embodiment of the invention, the SNE comprises 13.82 mole % cationic lipid, 80.07 mole % squalene, 3.43 mole % SPAN-85 and 2.68 mole % of PS-20 or PS-80.

In one embodiment of the invention, the SNE comprises 44.5 mole % cationic lipid, 51.56 mole % squalene, 2.21 mole % SPAN-85 and 1.72 mole % of PS-20 or PS-80.

In some embodiments of the invention, the SNE further comprises one or more non-cationic lipids which can be selected from a surfactant, a mixture of surfactants, a phospholipid, a terpene, a terpenoid, a triterpene or a combination thereof.

In some embodiments, the surfactant may include, but is not limited to: the polyoxyethylene sorbitan esters surfactants (commonly referred to as the Tweens), especially PS-20 and PS-80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span-85, Tween-85 or [2-[(2R,3S,41?)-4-hydroxy-3-[(Z)-octadec-9-enoyl]oxyoxolan-2-yl]-2-[(Z)-octadec-9-enoyl]oxyethyl] (Z)-octadec-9-enoate) and sorbitan monolaurate.

In some embodiments, mixtures of surfactants can be used, e.g. PS-20/Span 85 or PS-80/Span 85 mixtures. A combination of a polyoxyethylene sorbitan ester such as polyoxyethylene sorbitan monooleate (PS-80) and an octoxynol such as t-octylphenoxypolyethoxyethanol (Triton X-100) are also suitable. Another useful combination comprises laureth 9 plus a polyoxyethylene sorbitan ester and/or an octoxynol.

In some embodiments, the preferred amounts of surfactants or emulsifiers are: polyoxyethylene sorbitan esters (such as PS-20 or PS-80) 0.01 to 10 mole %, in particular about 1 to 4 mole %; octyl- or nonylphenoxy polyoxyethanols (such as Triton X-100, or other detergents in the Triton series) 0.001 to 10 mole %, in particular about 1 to 4 mole %; w/v, in particular 0.01 to 0.1% w/v; polyoxyethylene ethers (such as laureth 9) 0.1 to 20 mole %, preferably 0.5 to 10 mole % and in particular 1 to 4% mole % or about 10% by mass.

In some embodiments, the phospholipid may include, but is not limited to natural phospholipids including phosphatidylcholine (PC), phosphatidylethanolamine (PE), and phosphatidylglycerol (PG), phosphatidylserine (PS), phosphatidylinositol (PI), Phosphatidic acid (phosphatidate) (PA), dipalmintoylphosphatidylcholine, monoacyl-phosphatidylcholine (lyso PC), 1-palmitoyl-2-oleoyl-sn-glycero-3-phosphocholine (POPC), N-Acyl-PE, phosphoinositides, and phosphosphingolipids. Phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS). Fatty acids include C14:0, palmitic acid (C16:0), stearic acid (C18:0), oleic acid (C18:1), linoleic acid (C18:2), linolenic acid (C18:3), and arachidonic acid (C20:4), C20:0, C:22:0 and lethicin. In certain embodiments of the invention, the phospholipid may be phosphatidylserine, 1,2-Distearoyl-sn-glycero-3-phosphocholine (DSPC), 1,2-dipalmitoleoyl-sn-glycero-3-phosphocholine, 1,2-dimyristoyl-sn-glycero-3-phosphocholine (DMPC), dilauroyliphosphatidylcholine (DLPC), 1,2-dieicosenoyl-sn-glycero-3-phosphocholine, or 1,2-dioleoyl-sn-glycero-3-phosphocholine (DOPC).

In some embodiments, the terpine may include, but is not limited to monoterpenes including geraniol, terpineol, limonene, myrcene, linalool or pinene; sesquiterpenes including humulene, farnesenes, farnesol; diterpenes including cafestol, kahweol, cembrene and taxadiene, triterpenes including squalene and squalante; tetraterpenes including acyclic lycopene, the monocyclic gamma-carotene, and the bicyclic alpha- and beta-carotenes; polyterpenes and norisoprenoids. In some embodiments, the terpine is squalene.

In one embodiment of the invention, the SNE comprises 50-85 mole % squalene, and 1-10 mole % non-ionic surfactants. In one aspect of this embodiment, the non-ionic surfactant comprises a mixture of PS-20 and SPAN-85 or a mixture of PS-80 and SPAN-85.

In one embodiment of the invention, the SNE comprises 0-45 mole % cationic lipid, 50-85 mole % squalene, and 1-10 mol % non-ionic surfactants. In one aspect of this embodiment, the non-ionic surfactant comprises a mixture of PS-20 and SPAN-85 or a mixture of PS-80 and SPAN-85.

General Methods of Making SNEs (with and without a Cationic Lipid)

Generally, SNEs may be formed, for example, by initially combining and mixing lipid components together, or initially utilizing a single lipid, such as a cationic lipid. Once mixed and blended (when combining and mixing lipid components together), an aqueous buffer is added and mixed with the initial lipid or lipid components to form a blended emulsion mixture. The blended emulsion components are first subjected to course homogenization followed by fine homogenization. Then, the resulting formulation is subjected to a final filtration step and stored at 4° C. A lipid solution may include one or more cationic lipids, one or more terpenes (e.g., squalene), one or more sorbitan-based surfactants (e.g. PS-20 or PS-80; SPAN-85) at specific molar ratios.

### General Methods of Making LNPs

LNPs and methods of making LNPs are well known in the art. LNPs are known and described in the following publications: U.S. Pat. No. 7,691,405, US2006/0083780, US2006/0240554, US2008/0020058, US2009/0263407, US2009/0285881, WO2009/086558, WO2009/127060, WO2009/132131, WO2010/042877, WO2010/054384, WO2010/054401, WO2010/054405 and WO2010/054406.

A process of preparing an LNP consists of 4 primary steps: 1) solution preparation of a lipid mixture and an aqueous buffer; 2) LNP formation by means of split stream mixing; 3) ultra-filtration; and 4) filtration.

Generally, lipid components are dissolved in ethanol before being sterile filtered to form a lipid mixture. Several aqueous buffers are also prepared. A lipid mixture and buffer streams are then combined using a T-tube or Y mixer and then, immediately after exit, are diluted and mixed with an aqueous buffer to form an LNP intermediate. An LNP intermediate is then subjected to ultra-filtration to both concentrate the material as well as diafilter the material against a suitable buffer to remove residual ethanol. After the diafiltration, there is a final concentration step performed in order to achieve a final target concentration. LNP bulk is then filtered with a sterilizing filter and stored frozen at −70° C.

### Pneumococcal Conjugate Vaccine Compositions

Pneumococcal conjugate vaccines or compositions have been previously disclosed. See WO2011/100151, WO2019/139692 and WO2020/131763.

Example bacterial capsular polysaccharides from Streptococcus pneumoniae are serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38.

### General Methods for Making Capsular Polysaccharides

Methods of making pneumococcal conjugate vaccine compositions have been previously disclosed. See WO2011/100151, WO2019/139692 and WO2020/131763.

Bacterial capsular polysaccharides, particularly those that have been used as antigens, are suitable for use in the invention and can readily be identified by methods for identifying immunogenic and/or antigenic polysaccharides. Example bacterial capsular polysaccharides from Streptococcus pneumoniae are serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38.

Polysaccharides can be purified by known techniques. The invention is not limited to polysaccharides purified from natural sources, however, and the polysaccharides may be obtained by other methods, such as total or partial synthesis. Capsular polysaccharides from Streptococcus pneumoniae can be prepared by standard techniques known to those skilled in the art. For example, polysaccharides can be isolated from bacteria and may be sized to some degree by known methods (see, e.g., European Patent Nos. EP497524 and EP497525); and preferably by microfluidization accomplished using a homogenizer or by chemical hydrolysis. Streptococcus pneumoniae strains corresponding to each polysaccharide serotype may be grown in a soy-based medium. The individual polysaccharides may then be purified through standard steps including centrifugation, precipitation, and ultrafiltration. See, e.g., U.S. Patent Application Publication No. 2008/0286838 and U.S. Pat. No. 5,847,112. Polysaccharides can be sized in order to reduce viscosity and/or to improve filterability and the lot-to-lot consistency of subsequent conjugated products.

Purified polysaccharides can be chemically activated to introduce functionalities capable of reacting with a carrier protein using standard techniques. The chemical activation of polysaccharides and subsequent conjugation to a carrier protein are achieved by means described in U.S. Pat. Nos. 4,365,170, 4,673,574 and 4,902,506. Briefly, the pneumococcal polysaccharide is reacted with a periodate-based oxidizing agent such as sodium periodate, potassium periodate, or periodic acid resulting in oxidative cleavage of vicinal hydroxyl groups to generate reactive aldehyde groups. Suitable molar equivalents of periodate (e.g., sodium periodate, sodium metaperiodate and the like) include 0.05 to 0.5 molar equivalents (molar ratio of periodate to polysaccharide repeat unit) or 0.1 to 0.5 molar equivalents. The periodate reaction can be varied from 30 minutes to 24 hours depending on the diol conformation (e.g., acyclic diols, cis diols, trans diols), which controls accessibility of the reactive hydroxyl groups to the sodium periodate. The term “periodate” includes both periodate and periodic acid; the term also includes both metaperiodate (IO⁴⁻) and orthoperiodate (IO⁶⁻) and includes the various salts of periodate (e.g., sodium periodate and potassium periodate). Capsular polysaccharide may be oxidized in the presence of metaperiodate, or in the presence of sodium periodate (NaIO₄). Further, capsular polysaccharide may be oxidized in the presence of orthoperiodate, or in the presence of periodic acid.

Purified polysaccharides can also be connected to a linker. Once activated or connected to a linker, each capsular polysaccharide may be separately conjugated to a carrier protein to form a glycoconjugate. The polysaccharide conjugates may be prepared by known coupling techniques.

Polysaccharide can be coupled to a linker to form a polysaccharide-linker intermediate in which the free terminus of the linker is an ester group. The linker is therefore one in which at least one terminus is an ester group. The other terminus is selected so that it can react with the polysaccharide to form the polysaccharide-linker intermediate.

Polysaccharide can be coupled to a linker using a primary amine group in the polysaccharide. In this case, the linker typically has an ester group at both termini. This allows the coupling to take place by reacting one of the ester groups with the primary amine group in the polysaccharide by nucleophilic acyl substitution. The reaction results in a polysaccharide-linker intermediate in which the polysaccharide is coupled to the linker via an amide linkage. The linker is therefore a bifunctional linker that provides a first ester group for reacting with the primary amine group in the polysaccharide and a second ester group for reacting with the primary amine group in the carrier molecule. A typical linker is adipic acid N-hydroxysuccinimide diester (SIDEA).

The coupling can also take place indirectly, i.e. with an additional linker that is used to derivatize the polysaccharide prior to coupling to the linker.

Polysaccharide can be coupled to the additional linker using a carbonyl group at the reducing terminus of the polysaccharide. This coupling comprises two steps: (a1) reacting the carbonyl group with the additional linker; and (a2) reacting the free terminus of the additional linker with the linker. In these embodiments, the additional linker typically has a primary amine group at both termini, thereby allowing step (a1) to take place by reacting one of the primary amine groups with the carbonyl group in the polysaccharide by reductive amination. A primary amine group is used that is reactive with the carbonyl group in the polysaccharide. Hydrazide or hydroxylamino groups are suitable. The same primary amine group is typically present at both termini of the additional linker which allows for the possibility of polysaccharide (Ps)-Ps coupling. The reaction results in a polysaccharide-additional linker intermediate in which the polysaccharide is coupled to the additional linker via a C—N linkage.

Polysaccharide can be coupled to the additional linker using a different group in the polysaccharide, particularly a carboxyl group. This coupling comprises two steps: (a1) reacting the group with the additional linker; and (a2) reacting the free terminus of the additional linker with the linker. In this case, the additional linker typically has a primary amine group at both termini, thereby allowing step (a1) to take place by reacting one of the primary amine groups with the carboxyl group in the polysaccharide by EDAC activation. A primary amine group is used that is reactive with the EDAC-activated carboxyl group in the polysaccharide. A hydrazide group is suitable. The same primary amine group is typically present at both termini of the additional linker. The reaction results in a polysaccharide-additional linker intermediate in which the polysaccharide is coupled to the additional linker via an amide linkage.

### Carrier Protein

In a particular embodiment of the present invention, CRM197 is used as the carrier protein. CRM197 is a non-toxic variant (i.e., toxoid) of diphtheria toxin. CRM197 may be isolated from cultures of Corynebacterium diphtheria strain C7 (b197) grown in casamino acids and yeast extract-based medium. Further, CRM197 may be prepared recombinantly in accordance with the methods described in U.S. Pat. No. 5,614,382. Typically, CRM197 is purified through a combination of ultrafiltration, ammonium sulfate precipitation, and ion-exchange chromatography. In some embodiments, CRM197 is prepared in Pseudomonas fluorescens using Pfenex Expression Technology™ (Pfenex Inc., San Diego, CA).

Other suitable carrier proteins include additional inactivated bacterial toxins such as DT (Diphtheria toxoid), TT (tetanus toxoid) or fragment C of TT, pertussis toxoid, cholera toxoid (e.g., as described in International Patent Application Publication No. WO 2004/083251), E. coli LT, E. coli ST, and exotoxin A from Pseudomonas aeruginosa. Bacterial outer membrane proteins such as outer membrane complex c (OMPC), porins, transferrin binding proteins, pneumococcal surface protein A (PspA; See International Application Patent Publication No. WO 02/091998), pneumococcal surface adhesin protein (PsaA), C5a peptidase from Group A or Group B streptococcus, or Haemophilus influenzae protein D, pneumococcal pneumolysin (Kuo et a1., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; see, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et a1., 2001, Eur J Immunol 31:3816-3824) such as N19 protein (See Baraldoi et a1., 2004, Infect Immun 72:4884-7), iron uptake proteins (See International Patent Application Publication No. WO 01/72337), toxin A or B of C. difficile (See International Patent Publication No. WO 00/61761), and flagellin (See Ben-Yedidia et a1., 1998, Immunol Lett 64:9) can also be used as carrier proteins.

Where multivalent vaccines are used, a second carrier can be used for one or more of the antigens in a multivalent vaccine. The second carrier protein is preferably a protein that is non-toxic and non-reactogenic and obtainable in sufficient amount and purity. The second carrier protein is also conjugated or joined with an antigen, e.g., a Streptococcus pneumoniae polysaccharide to enhance immunogenicity of the antigen. Carrier proteins should be amenable to standard conjugation procedures. Each capsular polysaccharide not conjugated to a first carrier protein may be conjugated to the same second carrier protein (e.g., each capsular polysaccharide molecule being conjugated to a single carrier protein). Capsular polysaccharides not conjugated to a first carrier protein may be conjugated to two or more carrier proteins (each capsular polysaccharide molecule being conjugated to a single carrier protein). In such embodiments, each capsular polysaccharide of the same serotype is typically conjugated to the same carrier protein. Other DT mutants can be used as the second carrier protein, such as CRM176, CRM228, CRM45 (Uchida et a1., 1973, J Biol Chem 218:3838-3844); CRM9, CRM45 CRM102, CRM103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711.

Conjugation by Reductive Amination Covalent coupling of polysaccharide to carrier protein can be performed via reductive amination in which an amine-reactive moiety on the polysaccharide is directly coupled to primary amine groups (mainly lysine residues) of the protein. As is well known, a reductive amination reaction proceeds via a two-step mechanism. First, a Schiff base intermediate, of formula R—CH═N—R′, is formed by reaction of an aldehyde group on molecule 1 (R—CHO) with a primary amine group (R′—NH2) on molecule 2. In the second step, the Schiff base is reduced to form an amino compound of formula R—CH2-NH—R′. While many reducing agents are capable of being utilized, most often a highly selective reducing agent such as sodium cyanoborohydride (NaCNBH₃) is employed since such reagents will specifically reduce only the imine function of the Schiff base.

Since all the polysaccharides have an aldehyde function at the end of the chain (terminal aldehyde function), the conjugation methods comprising a reductive amination of the polysaccharide can be applied very generally and, when there is no other aldehyde function in the repeating unit (intrachain aldehyde function), such methods make it possible to obtain conjugates in which a polysaccharide molecule is coupled to a single molecule of carrier protein.

A typical reducing agent is cyanoborohydride salt such as sodium cyanoborohydride. The imine-selective reducing agent typically employed is sodium cyanoborohydride, although other cyanoborohydride salts can be used including potassium cyanoborohydride. Differences in starting cyanide levels in sodium cyanoborohydride reagent lots and residual cyanide in the conjugation reaction can lead to inconsistent conjugation performance, resulting in variable product attributes, such as conjugate size and conjugate Ps-to-CRM197 ratio. By controlling and/or reducing the free cyanide levels in the final reaction product, conjugation variability can be reduced.

Residual unreacted aldehydes on the polysaccharide are optionally reduced with the addition of a strong reducing agent, such as sodium borohydride. Generally, use of a strong reducing agent is preferred. However, for some polysaccharides, it is preferred to avoid this step. For example, Streptococcus pneumoniae serotype 5 contains a ketone group that may react readily with a strong reductant. In this case, it is preferable to bypass the reduction step to protect the antigenic structure of the polysaccharide.

Following conjugation, the polysaccharide-carrier protein conjugates are purified to remove excess conjugation reagents as well as residual free carrier protein and free polysaccharide by one or more of any techniques well known to the skilled artisan, including concentration/diafiltration operations, ultrafiltration, precipitation/elution, column chromatography, and depth filtration. See, e.g., U.S. Pat. No. 6,146,902. In one embodiment, the purifying step is by ultrafiltration.

### Pneumococcal Conjugate Compositions

The present invention provides pneumococcal conjugate compositions comprising, consisting essentially of, or alternatively, consisting of polysaccharide-carrier protein conjugates together with a SNE, with or without a cationic lipid. The present invention further provides pneumococcal conjugate compositions comprising, consisting essentially of, or alternatively, consisting of polysaccharide-carrier protein conjugates together with a SNE, with or without a cationic lipid and a pharmaceutically acceptable carrier. The present invention further provides pneumococcal conjugate compositions comprising, consisting essentially of, or alternatively, consisting of any of the polysaccharide-carrier protein conjugate combinations described herein together with a SNE, with or without a cationic lipid and optionally a pharmaceutically acceptable carrier. The compositions of the instant invention may comprise, consist essentially of, or consist of 2 to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 distinct polysaccharide-carrier protein conjugates, wherein each of the conjugates contains a different capsular polysaccharide conjugated to a carrier protein. In an embodiment, the polysaccharides that are a part of the polysaccharide-carrier protein conjugates, include, but are not limited to serotypes 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A or 20B), 22F, 23A, 23B, 23F, 24F, 33F, 35B, 35F, and 38 of Streptococcus pneumoniae. In another embodiment, the group of serotypes comprise, consist essentially of, or consist of 4, 6B, 9V, 14, 18C, 19F and 23F. In another embodiment, the group of serotypes comprise, consist essentially of or consist of 1, 3, 4, 5, 6A, 6B, 7F, 9V, 14, 18C, 19A, 19F and 23F. In another embodiment, the group of serotypes comprise, consist essentially of, or consist of 1, 3, 4, 5, 6A, 6B, 7F, 9V, 14, 18C, 19A, 19F, 22F, 23F and 33F. In another embodiment, the group of serotypes comprise, consist essentially of, or consist of 1, 3, 4, 5, 6A, 6B, 7F, 8, 9V, 10A, 11A, 12F, 14, 15B, 18C, 19A, 19F, 22F, 23F and 33F. In another embodiment, the group of serotypes comprise, consist essentially of, or consist of 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In another embodiment, the group of serotypes comprise, consist essentially of, or consist of 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B. In another embodiment, the group of serotypes comprise, consist essentially of, or consist of 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In another embodiment the group of serotypes comprise, consist essentially of, or consist of 3, 6A, 7F, 8, 9N, 10A, 11A, 12F, 15A, 15B, 16F, 17F, 19A, 20A, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, de-O-acetylated-15B, 16F, 17F, 19A, 20A, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15C, 16F, 17F, 19A, 20A, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15B, 16F, 17F, 19A, 20, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, de-O-acetylated-15B, 16F, 17F, 19A, 20, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15C, 16F, 17F, 19A, 20, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15B, 16F, 17F, 19A, 20B, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, de-O-acetylated-15B, 16F, 17F, 19A, 20B, 22F, 23A, 23B, 24F, 31, 33F and 35B. In another embodiment the group of serotypes comprise, consist essentially of, or consist of 3, 6A, 7F, 8, 9N, 10A, 11A, 12F, 15A, 15C, 16F, 17F, 19A, 20B, 22F, 23A, 23B, 24F, 31, 33F and 35B. In another embodiment the serotypes are conjugated to the carrier protein CRM197. In another embodiment, all serotypes are conjugated to a carrier protein. In another embodiment, the preferred carrier protein is CRM197.

### Methods of Use Dosage

The compositions of the present invention can be used to protect or treat a human susceptible to infection, e.g., a pneumococcal infection, by means of administering the vaccine via a systemic or mucosal route. In one embodiment, the present invention provides a method of inducing an immune response to a Streptococcus pneumoniae capsular polysaccharide conjugate, comprising administering to a human an immunologically effective amount of a composition of the present invention. In another embodiment, the present invention provides a method of vaccinating a human against a pneumococcal infection, comprising the step of administering to the human an immunologically effective amount of a composition of the present invention.

Optimal amounts of components for a particular vaccine can be ascertained by standard studies involving observation of appropriate immune responses in subjects. For example, in another embodiment, the dosage for human vaccination is determined by extrapolation from animal studies to human data. In another embodiment, the dosage is determined empirically.

The methods of the invention can be used for the prevention and/or reduction of primary clinical syndromes caused Streptococcus pneumonia, including both invasive infections (meningitis, pneumonia, and bacteremia), and noninvasive infections (acute otitis media, and sinusitis).

Administration of the compositions of the invention can include one or more of: injection via the intramuscular, intraperitoneal, intradermal or subcutaneous routes; or via mucosal administration to the oral/alimentary, respiratory or genitourinary tracts. In one embodiment, intranasal administration is used for the treatment of pneumonia or otitis media (as nasopharyngeal carriage of pneumococci can be more effectively prevented, thus attenuating infection at its earliest stage).

The amount of conjugate in each vaccine dose may be selected as an amount that induces an immunoprotective response without significant, adverse effects. Such amount can vary depending upon the pneumococcal serotype. Generally, for polysaccharide-based conjugates, each dose will comprise 0.1 to 100 mg of each polysaccharide, particularly 0.1 to 10 mg, and more particularly 1 to 5 mg. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 mg.

According to any of the methods of the present invention and in one embodiment, the subject is human. In certain embodiments, the human patient is an infant (less than 1 year of age), toddler (approximately 12 to 24 months), or young child (approximately 2 to 5 years). In other embodiments, the human patient is an elderly patient (>65 years). The compositions of this invention are also suitable for use with older children, adolescents and adults (e.g., aged 18 to 45 years or 18 to 65 years).

Accordingly, one embodiment of the invention includes a method of treating or preventing a disease or disorder caused by Streptococcus pneumonia in a patient or subject comprising administering to the subject an immunologically effective amount of any of the compositions of the invention. In further embodiments, the invention includes the compositions of the invention (i.e. the compositions described throughout the specification) (i) for use in, (ii) for use as a medicament or composition for, or (iii) for use in the preparation (or manufacture) of a medicament for: (a) therapy (e.g., of the human body); (b) medicine; (c) treatment or prophylaxis of infection by Streptococcus pneumonia; (e) prevention of recurrence of Streptococcus pneumonia infection; (f) reduction of the progression, onset or severity of pathological symptoms associated with Streptococcus pneumonia infection and/or reduction of the likelihood of a Streptococcus pneumonia infection or, (g) treatment, prophylaxis of, or delay in the onset, severity, or progression of Streptococcus pneumonia associated disease(s), including, but not limited to: meningitis, pneumonia, bacteremia, acute otitis media, and sinusitis, and treating or preventing disease or disorders caused by Streptococcus pneumonia.

In one embodiment of the methods of the present invention, a composition of the present invention is administered as a single inoculation. In another embodiment, the vaccine is administered twice, three times or four times or more, adequately spaced apart. For example, the composition may be administered at 1-, 2-, 3-, 4-, 5-, or 6-month intervals or any combination thereof. The immunization schedule can follow that designated for pneumococcal vaccines. For example, the routine schedule for infants and toddlers against invasive disease caused by Streptococcus pneumoniae is 2-, 4-, 6- and 12-15-months of age. Thus, in a preferred embodiment, the composition is administered as a 4-dose series at 2-, 4-, 6-, and 12-15-months of age.

The compositions of this invention may also include one or more proteins from Streptococcus pneumoniae. Examples of Streptococcus pneumoniae proteins suitable for inclusion include those identified in International Patent Application Publication Nos. WO 02/083855 and WO 02/053761.

### Inventive Formulations

In some embodiments, a composition is provided that includes an SNE and Streptococcus pneumoniae polysaccharide-carrier protein conjugates. In some embodiments, a composition is provided that includes an SNE and Streptococcus pneumoniae polysaccharide-carrier protein conjugates containing at least 1, or at least 3, or at least 7, or at least 10, or at least 13, or at least 15, or at least 20, or at least 24, or at least 27, or at least 30 Streptococcus pneumoniae serotypes. In some embodiments, a composition is provided that includes an SNE and Streptococcus pneumoniae polysaccharide-carrier protein conjugates containing 20, 21, 22, 23, 24, 25, 26, 27, 28, 29, 30, 31, 32, 33, 34, 35, 36, 37, 38, 39 or 40 Streptococcus pneumoniae serotypes. In some embodiments, a composition is provided that includes an SNE and Streptococcus pneumoniae polysaccharide-carrier protein conjugates containing Streptococcus pneumoniae serotypes consisting of 4, 6B, 9V, 14, 18C, 19F and 23F. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 9V, 14, 18C, 19A, 19F and 23F. In some embodiments, a composition is provided that includes a SNE and Streptococcus pneumoniae polysaccharide-carrier protein conjugates containing Streptococcus pneumoniae serotypes consisting of 1, 3, 4, 5, 6A, 6B, 7F, 9V, 14, 18C, 19A, 19F, 22F, 23F and 33F. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 8, 9V, 10A, 11A, 12F, 14, 15B, 18C, 19A, 19F, 22F, 23F and 33F. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 9V, 10A, 12F, 14, 15A, 15B, 18C, 19A, 19F, 22F, 23B, 23F, 24F, 33F and 35B. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 9V, 10A, 12F, 14, 15A, de-O-acetyl-15B, 18C, 19A, 19F, 22F, 23B, 23F, 24F, 33F and 35B. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 9V, 10A, 12F, 14, 15A, 15C, 18C, 19A, 19F, 22F, 23B, 23F, 24F, 33F and 35B. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 9V, 10A, 12F, 14, 15A, 15B, 18C, 19A, 19F, 22F, 23B, 23F, 24F, 33F and 35B. In some embodiments a composition is provided that includes an SNE and Streptococcus pneumoniae polysaccharide-carrier protein conjugates containing Streptococcus pneumoniae serotypes consisting of 1, 3, 4, 5, 6A, 6B, 7F, 9V, 10A, 12F, 14, 15A, de-O-acetyl-15B, 18C, 19A, 19F, 22F, 23B, 23F, 24F, 33F and 35B. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 9V, 10A, 12F, 14, 15A, 15C, 18C, 19A, 19F, 22F, 23B, 23F, 24F, 33F and 35B. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 8, 9V, 10A, 11A, 12F, 14, 15A, de-O-acetyl-15B, 18C, 19A, 19F, 22F, 23B, 23F, 24F, 33F and 35B. In some embodiments, a composition is provided that includes an SNE and Streptococcus pneumoniae polysaccharide-carrier protein conjugates containing Streptococcus pneumoniae serotypes consisting of 1, 3, 4, 5, 6A, 6B, 7F, 8, 9V, 10A, 11A, 12F, 14, 15A, 15C, 18C, 19A, 19F, 22F, 23B, 23F, 24F, 33F and 35B. In some embodiments, a composition is provided that includes an SNE and Streptococcus pneumoniae polysaccharide-carrier protein conjugates containing Streptococcus pneumoniae serotypes consisting of 3, 6A, 7F, 8, 9N, 10A, 11A, 12F, 15A, 15B, 16F, 17F, 19A, 20A, 22F, 23A, 23B, 24F, 31, 33F and 35B. In some embodiments, a composition is provided that includes a SNE and Streptococcus pneumoniae polysaccharide-carrier protein conjugates containing Streptococcus pneumoniae serotypes consisting of 3, 6A, 7F, 8, 9N, 10A, 11A, 12F, 15A, de-O-acetylated-15B, 16F, 17F, 19A, 20A, 22F, 23A, 23B, 24F, 31, 33F and 35B. In some embodiments, a composition is provided that includes an SNE and Streptococcus pneumoniae polysaccharide-carrier protein conjugates containing Streptococcus pneumoniae serotypes consisting of 3, 6A, 7F, 8, 9N, 10A, 11A, 12F, 15A, 15C, 16F, 17F, 19A, 20A, 22F, 23A, 23B, 24F, 31, 33F and 35B. In some embodiments, a composition is provided that includes an SNE and Streptococcus pneumoniae polysaccharide-carrier protein conjugates containing Streptococcus pneumoniae serotypes consisting of 3, 6A, 7F, 8, 9N, 10A, 11A, 12F, 15A, 15B, 16F, 17F, 19A, 20, 22F, 23A, 23B, 24F, 31, 33F and 35B. In some embodiments, a composition is provided that includes an SNE and Streptococcus pneumoniae polysaccharide-carrier protein conjugates containing Streptococcus pneumoniae serotypes consisting of 3, 6A, 7F, 8, 9N, 10A, 11A, 12F, 15A, de-O-acetylated-15B, 16F, 17F, 19A, 20, 22F, 23A, 23B, 24F, 31, 33F and 35B. In some embodiments, a composition is provided that includes an SNE and Streptococcus pneumoniae polysaccharide-carrier protein conjugates containing Streptococcus pneumoniae serotypes consisting of 3, 6A, 7F, 8, 9N, 10A, 11A, 12F, 15A, 15C, 16F, 17F, 19A, 20, 22F, 23A, 23B, 24F, 31, 33F and 35B. In some embodiments, a composition is provided that includes an SNE and Streptococcus pneumoniae polysaccharide-carrier protein conjugates containing Streptococcus pneumoniae serotypes consisting of 3, 6A, 7F, 8, 9N, 10A, 11A, 12F, 15A, 15B, 16F, 17F, 19A, 20B, 22F, 23A, 23B, 24F, 31, 33F and 35B. In some embodiments, a composition is provided that includes an SNE and Streptococcus pneumoniae polysaccharide-carrier protein conjugates containing Streptococcus pneumoniae serotypes consisting of 3, 6A, 7F, 8, 9N, 10A, 11A, 12F, 15A, de-O-acetylated-15B, 16F, 17F, 19A, 20B, 22F, 23A, 23B, 24F, 31, 33F and 35B. In some embodiments, a composition is provided that includes an SNE and Streptococcus pneumoniae polysaccharide-carrier protein conjugates containing Streptococcus pneumoniae serotypes consisting of 3, 6A, 7F, 8, 9N, 10A, 11A, 12F, 15A, 15C, 16F, 17F, 19A, 20B, 22F, 23A, 23B, 24F, 31, 33F and 35B.

The present invention further provides a pneumococcal conjugate composition comprising Streptococcus pneumoniae polysaccharide-carrier protein conjugates and a stable nanoemulsion (SNE).

The present invention further provides a pneumococcal conjugate composition comprising Streptococcus pneumoniae polysaccharide-carrier protein conjugates and an SNE, and a pharmaceutically acceptable carrier.

The present invention further provides a pneumococcal conjugate composition comprising Streptococcus pneumoniae polysaccharide-carrier protein conjugates and an SNE comprising an emulsifier and/or a solubilizer and/or a surfactant and optionally a cationic lipid, or mixtures thereof.

The present invention further provides a pneumococcal conjugate composition comprising Streptococcus pneumoniae polysaccharide-carrier protein conjugates and an SNE comprising surfactants and/or terpenes and/or cationic lipids or mixtures thereof.

The present invention further provides a pneumococcal conjugate composition comprising Streptococcus pneumoniae polysaccharide-carrier protein conjugates and an SNE comprising one or more surfactants and one or more terpenes and optionally one or more cationic lipids.

The present invention further provides a pneumococcal conjugate composition comprising Streptococcus pneumoniae polysaccharide-carrier protein conjugates and an SNE comprising one to three surfactants and one to three terpenes and optionally one to three cationic lipids.

The present invention further provides a pneumococcal conjugate composition comprising Streptococcus pneumoniae polysaccharide-carrier protein conjugates and an SNE comprising one to two surfactants and one to two terpenes and optionally one to two cationic lipids.

The present invention provides a pneumococcal conjugate composition comprising 1) Streptococcus pneumoniae polysaccharide-carrier protein conjugates and 2) an SNE comprising i) sorbitan trioleate (SPAN-85); (ii) polysorbate-20 (PS-20) or polysorbate-80 (PS-80); iii) squalene; and an optional iv) cationic lipid.

The present invention provides a pneumococcal conjugate composition comprising 1) Streptococcus pneumoniae polysaccharide-carrier protein conjugates and 2) an SNE comprising i) sorbitan trioleate (SPAN-85); (ii) polysorbate-20 (PS-20); iii) squalene; and an optional iv) cationic lipid.

The present invention further provides a pneumococcal conjugate composition comprising 1) Streptococcus pneumoniae polysaccharide-carrier protein conjugates and 2) an SNE comprising i) sorbitan trioleate (SPAN-85); (ii) polysorbate-20 (PS-20) or polysorbate-80 (PS-80); iii) squalene; and an optional iv) cationic lipid, wherein the cationic lipid is not associated with a lipid nanoparticle (LNP).

The present invention further provides a pneumococcal conjugate composition comprising 1) Streptococcus pneumoniae polysaccharide-carrier protein conjugates and 2) an SNE comprising i) sorbitan trioleate (SPAN-85); (ii) polysorbate-20 (PS-20); iii) squalene; and an optional iv) cationic lipid, wherein the cationic lipid is not associated with a lipid nanoparticle (LNP).

The present invention further provides a pneumococcal conjugate composition, as described above, and a pharmaceutically acceptable carrier.

In some embodiments, the compositions described above comprise polysaccharide-carrier protein conjugates wherein the carrier protein CRM197.

In an embodiment of the compositions above, the SNE comprises PS-20. In another embodiment, the SNE comprises PS-80.

In an embodiment, the composition comprises the cationic lipid CLA, CLX, or CLY.

In an embodiment, the composition comprises the cationic lipid CLA.

In an embodiment, the composition comprises a cationic lipid selected from DLinDMA, DLinKC2DMA, DLin-MC3-DMA, CLinDMA, and S-Octyl CLinDMA.

In one embodiment, the Streptococcus pneumoniae polysaccharide-carrier protein conjugates contain a polysaccharide of a particular Streptococcus pneumonia serotype which include, but are not limited to, any of the serotypes selected from 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F and 38 conjugated to a carrier protein which is CRM197. In another embodiment, the Streptococcus pneumoniae polysaccharide-carrier protein conjugates comprise, consist essentially of, or consist of serotypes 4, 6B, 9V, 14, 18C, 19F and 23F, all conjugated to the carrier protein CRM197. In another embodiment, the serotypes comprise, consist essentially of, or consist of serotypes 1, 3, 4, 5, 6A, 6B, 7F, 9V, 14, 18C, 19A, 19F and 23F, all conjugated to the carrier protein CRM197. In another embodiment, the serotypes comprise, consist essentially of, or consist of serotypes 1, 3, 4, 5, 6A, 6B, 7F, 9V, 14, 18C, 19A, 19F, 22F, 23F and 33F, all conjugated to the carrier protein CRM197. In another embodiment, the serotypes comprise, consist essentially of, or consist of serotypes 1, 3, 4, 5, 6A, 6B, 7F, 8, 9V, 10A, 11A, 12F, 14, 15B, 18C, 19A, 19F, 22F, 23F and 33F, all conjugated to the carrier protein CRM197. In another embodiment, the serotypes comprise, consist essentially of, or consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, all conjugated to the carrier protein CRM197. In another embodiment, the serotypes comprise, consist essentially of, or consist of serotypes 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B, all conjugated to the carrier protein CRM197. In another embodiment, the serotypes comprise, consist essentially of, or consist of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, 15B, 16F, 17F, 19A, 20A, 22F, 23A, 23B, 24F, 31, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, de-O-acetylated-15B, 16F, 17F, 19A, 20A, 22F, 23A, 23B, 24F, 31, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, 15C, 16F, 17F, 19A, 20A, 22F, 23A, 23B, 24F, 31, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, 15B, 16F, 17F, 19A, 20, 22F, 23A, 23B, 24F, 31, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, de-O-acetylated-15B, 16F, 17F, 19A, 20, 22F, 23A, 23B, 24F, 31, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, 15C, 16F, 17F, 19A, 20, 22F, 23A, 23B, 24F, 31, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, 15B, 16F, 17F, 19A, 20B, 22F, 23A, 23B, 24F, 31, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, de-O-acetylated-15B, 16F, 17F, 19A, 20B, 22F, 23A, 23B, 24F, 31, 33F and 35B, all conjugated to the carrier protein CRM197. In another embodiment the serotypes comprise, consist essentially of, or consist of serotypes 3, 6A, 7F, 8, 9N, 10A, 11A, 12F, 15A, 15C, 16F, 17F, 19A, 20B, 22F, 23A, 23B, 24F, 31, 33F and 35B, all conjugated to the carrier protein CRM197.

In an embodiment, the present invention provides a pneumococcal conjugate composition comprising 7 distinct Streptococcus pneumoniae polysaccharide-carrier protein conjugates, wherein the Streptococcus pneumoniae polysaccharides consist of serotypes 4, 6B, 9V, 14, 18C, 19F and 23F, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 13 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F and 23F, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 15 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 20 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15B, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, de-O-acetylated-15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15C, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); and squalene.

In an embodiment, the present invention provides a pneumococcal conjugate composition comprising 7 distinct Streptococcus pneumoniae polysaccharide-carrier protein conjugates, wherein the Streptococcus pneumoniae polysaccharides consist of serotypes 4, 6B, 9V, 14, 18C, 19F and 23F, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 13 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F and 23F, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 15 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 20 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15B, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene and a cationic lipid.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, de-O-acetylated-15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene; and a cationic lipid.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15C, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene and a cationic lipid.

In an embodiment, the present invention provides a pneumococcal conjugate composition comprising 7 distinct Streptococcus pneumoniae polysaccharide-carrier protein conjugates, wherein the Streptococcus pneumoniae polysaccharides consist of serotypes 4, 6B, 9V, 14, 18C, 19F and 23F, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 13 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F and 23F, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 15 distinct Streptococcus pneumoniae polysaccharide-carrier protein conjugates, wherein the Streptococcus pneumoniae polysaccharides consist of serotypes 1, 3, 4, 5, 6A, 6B, 7F, 9V, 14, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 20 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15B, 18C, 19A, 19F, 22F, 23F and 33F, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene and the cationic lipid CLA.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 24 distinct Streptococcus pneumoniae polysaccharide-carrier protein conjugates, wherein the Streptococcus pneumoniae polysaccharides consist of serotypes 1, 3, 4, 5, 6A, 6B, 7F, 8, 9V, 10A, 11A, 12F, 14, 15A, 15C, 18C, 19A, 19F, 22F, 23B, 23F, 24F, 33F and 35B, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, de-O-acetylated-15B, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene; and the cationic lipid CLA.

In an embodiment, the present invention provides a pneumococcal conjugate composition comprising 21 distinct Streptococcus pneumoniae polysaccharide-carrier protein conjugates, wherein the Streptococcus pneumoniae polysaccharides consist of serotypes 3, 6A, 7F, 8, 9N, 10A, 11A, 12F, 15A, 15C, 16F, 17F, 19A, 20, 22F, 23A, 23B, 24F, 31, 33F and 35B, all serotypes conjugated to the carrier protein CRM197, and further comprising sorbitan trioleate (SPAN-85); polysorbate-20 (PS-20) or polysorbate-80 (PS-80); squalene and the cationic lipid CLA.

In an embodiment of the compositions above, the SNE comprises PS-20. In another embodiment, the SNE comprises PS-80.

In some embodiments, a composition is provided that includes about 2 μg/mL to about 400 mg/mL cationic lipid and at least one Streptococcus pneumoniae polysaccharide-carrier protein conjugate, wherein each of the conjugates is present in a concentration of about 0.02 μg/mL to about 200 μg/mL.

In some embodiments, a composition is provided that includes about 0.04 μg/mL to about 80 mg/mL cationic lipid and at least one Streptococcus pneumoniae polysaccharide-carrier protein conjugate, wherein each of the conjugates is present in a concentration of about 0.004 μg/mL to about 40 μg/mL.

In some embodiments, a composition is provided that includes about 100 μg/mL to about 4.2 mg/mL cationic lipid and at least one Streptococcus pneumoniae polysaccharide-carrier protein conjugate, wherein each of the conjugates is present in a concentration of about 0.004 μg/mL to about 40 μg/mL.

In some embodiments, a composition is provided that includes about 100 μg/mL to about 20 mg/mL cationic lipid and at least one Streptococcus pneumoniae polysaccharide-carrier protein conjugate, wherein each of the conjugates is present in a concentration of about 0.004 μg/mL to about 40 μg/mL.

In some embodiments, a composition is provided that includes about 2 μg/mL to about 400 mg/mL cationic lipid and at least one Streptococcus pneumoniae polysaccharide-carrier protein conjugate, wherein each of the conjugates is present in a concentration of about 0.02 μg/mL to about 200 μg/mL prepared as a co-lyophilized formulation.

In some embodiments, a composition is provided that includes about 2 μg/mL to about 400 mg/mL cationic lipid, 2 μg/mL to about 2 mg/mL of Aluminum in the form of APA and at least one Streptococcus pneumoniae polysaccharide-carrier protein conjugate, wherein each of the conjugates is present in a concentration of about 0.02 μg/mL to about 200 μg/mL prepared as a co-lyophilized formulation.

In some embodiments, a composition is provided that includes about 20 μg/mL to about 2.4 mg/mL cationic lipid and at least one Streptococcus pneumoniae polysaccharide-carrier protein conjugate, wherein each of the conjugates is present in a concentration of about 0.2 μg/mL to about 24 μg/mL.

In some embodiments, a composition is provided that includes about 60 μg/mL to about 2.4 mg/mL cationic lipid and at least one Streptococcus pneumoniae polysaccharide-carrier protein conjugate, wherein each of the conjugates is present in a concentration of about 0.2 μg/mL to about 24 μg/mL.

In some embodiments, a composition, as highlighted in the various embodiments above, is provided that includes about 0.1 μg/mL to about 400 mg/mL cationic lipid, and further includes SPAN-85, PS-20 or PS-80 and squalene. In another embodiment, the cationic lipid is CLA. In another embodiment, the cationic lipid is CLX. In another embodiment, the cationic lipid is CLY.

In some embodiments, a composition, as highlighted in the various embodiments above, is provided that includes about 60 μg/mL to about 2.4 mg/mL cationic lipid, and further includes 6 μg/mL-240 μg/mL SPAN-85, 6 μg/mL-240 μg/mL PS-20 or PS-80 and 60 μg/mL-2.4 mg/mL of squalene. In another embodiment, the cationic lipid is CLA. In another embodiment, the cationic lipid is CLX. In another embodiment, the cationic lipid is CLY.

In some embodiments, a composition is provided that includes about 6 μg/mL-24 mg/mL SPAN-85, 6 μg/mL-24 mg/mL PS-20 or PS-80 and 60 μg/mL-240 mg/mL of squalene and at least one Streptococcus pneumoniae polysaccharide-carrier protein conjugate, wherein each of the conjugates is present in a concentration of about 0.02 μg/mL to about 200 g/mL.

In some embodiments, a composition is provided that includes about 6 μg/mL-24 mg/mL SPAN-85, 6 μg/mL-24 mg/mL PS-20 or PS-80 and 60 μg/mL-240 mg/mL of squalene and at least one Streptococcus pneumoniae polysaccharide-carrier protein conjugate, wherein each of the conjugates is present in a concentration of about 0.004 μg/mL to about 40 g/mL.

In some embodiments, a composition is provided that includes about 6 μg/mL-24 mg/mL SPAN-85, 6 μg/mL-24 mg/mL PS-20 or PS-80 and 60 μg/mL-240 mg/mL of squalene and at least one Streptococcus pneumoniae polysaccharide-carrier protein conjugate, wherein each of the conjugates is present in a concentration of about 0.004 μg/mL to about 40 g/mL.

In some embodiments, a composition is provided that includes about 2 μg/mL-24 mg/mL SPAN-85, 2 μg/mL-2.4 mg/mL PS-20 or PS-80 and 20 μg/mL-24 mg/mL of squalene and at least one Streptococcus pneumoniae polysaccharide-carrier protein conjugate, wherein each of the conjugates is present in a concentration of about 0.004 μg/mL to about 40 g/mL.

In some embodiments, a composition is provided that includes about 6 μg/mL-2.4 mg/mL SPAN-85, 6 μg/mL-2.4 mg/mL PS-20 or PS-80 and 60 μg/mL-24 mg/mL of squalene and at least one Streptococcus pneumoniae polysaccharide-carrier protein conjugate, wherein each of the conjugates is present in a concentration of about 0.02 μg/mL to about 200 g/mL prepared as a co-lyophilized formulation.

In some embodiments, a composition is provided that includes about 20 μg/mL to about 2.4 mg/mL cationic lipid and at least one Streptococcus pneumoniae polysaccharide-carrier protein conjugate, wherein each of the conjugates is present in a concentration of about 0.2 μg/mL to about 24 μg/mL.

In some embodiments, a composition is provided that includes 6 μg/mL-2.4 mg/mL SPAN-85, 6 μg-2.4 mg/mL PS-20 or PS-80 and 60 μg/mL-24 mg/mL of squalene and at least one Streptococcus pneumoniae polysaccharide-carrier protein conjugate, wherein each of the conjugates is present in a concentration of about 0.2 μg/mL to about 24 μg/mL.

In some embodiments, a composition is provided that includes 6 μg/mL-2.4 mg/mL SPAN-85, 6 μg/mL-2.4 mg/mL PS-20 or PS-80 and 60 μg/mL-24 mg/mL of squalene and at least one Streptococcus pneumoniae polysaccharide-carrier protein conjugate, wherein each of the conjugates is present in a concentration of about 0.2 μg/mL to about 24 μg/mL.

In some embodiments, a composition, as highlighted in the various embodiments above and includes 6 μg/mL-24 mg/mL SPAN-85, 6 μg-24 mg/mL PS-20 or PS-80 and 60 μg/mL-240 mg/mL of squalene.

In some embodiments, a composition, as highlighted in the various embodiments above and includes 6 μg/mL-2.4 mg/mL SPAN-85, 6 μg/mL-2.4 mg/mL PS-20 or PS-80 and 60 μg/mL-24 mg/mL of squalene.

In some embodiments, a composition, as highlighted in the various embodiments above, is provided that includes about 30 μg/mL to about 2.4 mg/mL cationic lipid, and further includes 6 μg/mL-14 mg/mL SPAN-85, 6 μg/mL-14 mg/mL PS-20 or PS-80 and 60 μg/mL-34 mg/mL of squalene. In another embodiment, the cationic lipid is CLA. In another embodiment, the cationic lipid is CLX. In another embodiment, the cationic lipid is CLY.

In some embodiments, a composition, as highlighted in the various embodiments above, is provided that includes about 60 μg/mL to about 2.4 mg/mL cationic lipid, and further includes 6 μg/mL-14 mg/mL SPAN-85, 6 μg/mL-14 mg/mL PS-20 or PS-80 and 60 μg/mL-34 mg/mL of squalene. In another embodiment, the cationic lipid is CLA. In another embodiment, the cationic lipid is CLX. In another embodiment, the cationic lipid is CLY.

In some embodiments, a composition is provided that includes 6 μg/mL-14 mg/mL SPAN-85, 6 μg/mL-14 mg/mL PS-20 or PS-80 and 60 μg/mL-34 mg/mL of squalene and at least one Streptococcus pneumoniae polysaccharide-carrier protein conjugate, wherein each of the conjugates is present in a concentration of about 0.2 μg/mL to about 24 μg/mL.

Compositions of the present invention may be administered subcutaneously, topically, orally, on the mucosa, intravenously, or intramuscularly. The compositions are administered in an amount sufficient to elicit a protective response. Compositions can be administered by various routes, for example, orally, parenterally, subcutaneously, on the mucosa, or intramuscularly. The dose administered may vary depending on the general condition, sex, weight and age of the patient, and the route of administration.

Compositions of the present invention, as highlighted in the various embodiments above, may be referred to as immunogenic compositions.

Compositions of the present invention, as highlighted in the various embodiments above may be referred to as vaccines or vaccine compositions.

In an embodiment, a composition is provided, wherein the SNE comprises PS-20, sorbitan trioleate, squalene and (13Z, 16Z)—N, N-dimethyl-3-nonyldocosa 13, 16-dien-1-amine.

In an embodiment, a composition is provided, wherein the SNE comprises 5-15 mol % sorbitan trioleate, 25-35 mole % PS-20 or PS-80, 1-2.5 mol % squalene, and 55-65 mol % (13Z, 16Z)—N, N-dimethyl-3-nonyldocosa 13, 16-dien-1-amine.

In an embodiment, a composition is provided, wherein the SNE, including the cationic lipid, comprises up to 75 mol % of cationic lipid, up to 30 mol % of sorbitan trioleate, up to 30 mol % of polysorbate-20 or polysorbate-80 and 25-85 mol % amount squalene.

In an embodiment, a composition is provided, wherein the SNE, including the cationic lipid, comprises up to 50 mol % of cationic lipid, up to 10 mol % of sorbitan trioleate, up to 10 mol % of polysorbate-20 or polysorbate-80 and 50-80 mol % amount squalene.

In an embodiment, a composition is provided, wherein the SNE, including the cationic lipid, comprises up to 24 mol % of cationic lipid, 1-8 mol % of sorbitan trioleate, 1-8 mol % of polysorbate-20 or polysorbate-80 and 60-75 mol % amount squalene.

In an embodiment, a composition is provided, wherein the SNE, including the cationic lipid, comprises about 10-14 mol % of cationic lipid, 1-4 mol % of sorbitan trioleate, 1-4 mol % of polysorbate-20 or polysorbate-80 and 50-80 mol % amount squalene.

In an embodiment, a composition is provided, wherein the SNE, including the cationic lipid, comprises 30-65 mol % cationic lipid, 5-30 mol % sorbitan trioleate, 10-40 mol % squalene, and 0.5-4 mol % PS-20 or PS-80.

In an embodiment, a composition is provided, wherein the SNE, including the cationic lipid, comprises 55-65 mol % cationic lipid, 5-15 mol % sorbitan trioleate, 25-35 mol % squalene, and 1-2.5 mol % PS-20 or PS-80.

In an embodiment, a composition is provided, wherein the SNE, including the cationic lipid, comprises 13-45 mol % cationic lipid, 2-4 mol % sorbitan trioleate, 50-82 mol % squalene, and 1.5-3 mol % PS-20 or PS-80.

In an embodiment, a composition is provided, wherein the SNE, including the cationic lipid, comprises 13-14 mol % cationic lipid, 1-2 mol % sorbitan trioleate, 79-81 mol % squalene, and 1-2 mol % PS-20 or PS-80.

In an embodiment, a composition is provided, wherein the SNE, including the cationic lipid, comprises 0 mol % cationic lipid, 8-10 mol % sorbitan trioleate, 80-84 mol % squalene, and 8-10 mol % PS-20 or PS-80.

In an embodiment, a composition is provided, wherein the SNE, including the cationic lipid, comprises 20 mol % cationic lipid, 30 mol % sorbitan trioleate, 20 mol % squalene, and 30 mol % PS-20 or PS-80.

In an embodiment, a composition is provided, wherein the SNE, including the cationic lipid, comprises about 2 mol % cationic lipid, about 8 mol % sorbitan trioleate, about 82 mol % squalene, and about 8 mol % PS-20 or PS-80.

In an embodiment, a composition is provided, wherein the SNE, including the cationic lipid, comprises 2 mol % cationic lipid, 8 mol % sorbitan trioleate, 82 mol % squalene, and 8 mol % PS-20 or PS-80.

In an embodiment, a composition is provided, wherein the SNE, including the cationic lipid, comprises about 13.82 mol % cationic lipid, about 3.43 mol % sorbitan trioleate, about 80.07 mol % squalene, and about 2.68 mol % PS-20 or PS-80.

In an embodiment, a composition is provided, wherein the SNE, including the cationic lipid, comprises 13.82 mol % cationic lipid, 3.43 mol % sorbitan trioleate, 80.07 mol % squalene, and 2.68 mol % PS-20 or PS-80.

In an embodiment, a composition is provided, wherein the SNE, including the cationic lipid, comprises about 44.5 mol % cationic lipid, about 2.21 mol % sorbitan trioleate, about 51.56 mol % squalene, and about 1.72 mol % PS-20 or PS-80.

In an embodiment, a composition is provided, wherein the SNE, including the cationic lipid, comprises 44.5 mol % cationic lipid, 2.21 mol % sorbitan trioleate, 51.56 mol % squalene, and 1.72 mol % PS-20 or PS-80.

In an embodiment, a composition is provided, wherein the SNE, comprises 32 mole % squalene, 34 mole % SPAN-85 and 34 mole % of PS-20 or PS-80.

In an embodiment, a composition is provided, wherein the SNE, comprises 98 mole % squalene, 1 mole % SPAN-85 and 1 mole % of PS-20 or PS-80.

In an embodiment, a composition is provided, wherein the SNE, comprises 86 mole % squalene, 7 mole % SPAN-85 and 7 mole % of PS-20 or PS-80.

In an embodiment, a composition is provided, wherein the SNE, comprises 92 mole % squalene, 4 mole % SPAN-85 and 4 mole % of PS-20 or PS-80.

In an embodiment, a composition is provided, wherein the SNE, comprises 94 mole % squalene, 3 mole % SPAN-85 and 3 mole % of PS-20 or PS-80.

In an embodiment, a composition is provided, wherein the SNE, comprises 92.91 mole % squalene, 3.98 mole % SPAN-85 and 3.11 mole % of PS-20 or PS-80.

In an embodiment, a composition is provided, wherein the SNE, comprises 62 mole % squalene, 17 mole % SPAN-85 and 17 mole % of PS-20 or PS-80.

In each of the embodiments described above the composition further comprises Streptococcus pneumoniae polysaccharide-carrier protein conjugates.

The present invention provides methods of treating or preventing pneumococcal diseases by administration of the compositions described above.

The present invention provides the use of the compositions described above for treating or preventing pneumococcal diseases.

All publications mentioned herein are incorporated by-reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be used by one skilled in the art without departing from the scope or spirit of the invention as defined in the appended claims.

The following examples illustrate, but do not limit the invention.

### EXAMPLES

### Example 1: Preparation of Pneumococcal Polysaccharide-Carrier Protein Conjugates Using DMSO Conjugation

Polysaccharide(s) (as highlighted below and in the Tables and Examples) was dissolved, sized to a target molecular mass, chemically activated and buffer-exchanged by ultrafiltration. Activated polysaccharide and purified CRM197 were individually lyophilized and re-dissolved in DMSO. Re-dissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Size Reduction

Purified pneumococcal capsular polysaccharide (otherwise termed “Ps”) powder was dissolved in water. With the exception of ST-19A (serotype is otherwise termed “ST”) which was not sized reduced, dissolved polysaccharide was 0.45-micron filtered and either homogenized or acid hydrolyzed to reduce the molecular mass of the Ps. Target Ps size was achieved for homogenization by controlling the pressure and number of passes. Target Ps size was achieved for acid hydrolysis by controlling the temperature and time. Polysaccharide was then 0.2-micron filtered and concentrated and diafiltered against water using a 5 or 10 kDa NMWCO tangential flow ultrafiltration membrane.

### De-O-Acetylation

Size reduced ST-15B Ps solution was heated to 60° C. and sodium bicarbonate pH 9.4 buffer was added to a final concentration of 50 mM. The batch was incubated at 60° C. to release 0-acetyl groups. Potassium phosphate pH 6 buffer was added to neutralize pH and the solution was cooled to ambient temperature. The solution was then concentrated and diafiltered against water using a 5 or 10 kDa NMWCO tangential flow ultrafiltration membrane.

### Deketalization (St-4 Only)

Sized reduced ST-4 Ps solution was adjusted to 50° C. and pH 4.1 with a sodium acetate buffer to partially deketalize the polysaccharide. The polysaccharide solution was then cooled to 22° C. prior to activation.

### Polysaccharide Oxidation

The polysaccharide solution was adjusted to 22° C. for all serotypes, except for ST-5, 7F and 19F, which were adjusted to 4° C. The solution was also adjusted to pH 4-5 with a sodium acetate buffer to minimize polysaccharide size reduction due to activation. Polysaccharide activation was initiated with the addition of a sodium metaperiodate solution. The amount of sodium metaperiodate added was controlled to achieve a target level of polysaccharide activation (moles aldehyde per mole of polysaccharide repeating unit).

The activated product for all serotypes except ST-5 was diafiltered against 10 mM potassium phosphate, pH 6.4 followed by diafiltration against water using a 5 or 10 kDa NMWCO tangential flow ultrafiltration membrane. For ST-5, the activated product was diafiltered against 10 mM sodium acetate, pH 4.1 followed by diafiltration against water using a 5 kDa NMWCO tangential flow ultrafiltration membrane. Ultrafiltration was conducted at 2-8° C. for all serotypes.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered. Activated polysaccharides were formulated for lyophilization with water and sucrose. CRM197 was formulated for lyophilization at 6 mg Pr/mL (the CRM197 carrier protein is otherwise referred to as “Pr”) with sucrose concentration of 1% w/v. Formulated Ps and CRM197 solutions were individually lyophilized. Lyophilized Ps and CRM197 materials were re-dissolved individually in equal volumes of DMSO. Additives such as salt were spiked into the Ps-DMSO for some serotypes. The polysaccharide and CRM197 solutions were blended to achieve a target polysaccharide concentration and polysaccharide to CRM197 mass ratio. The mass ratio was selected to control the polysaccharide to CRM197 ratio in the resulting conjugate. A reducing agent such as sodium cyanoborohydride was added for most serotypes and conjugation proceeded at 22° C.

### Final Reduction

A reducing agent such as sodium borohydride was added following the conjugation reaction and incubated at 22° C. for all serotypes. The batch was diluted into 150 mM sodium chloride, with approximately 0.025% (w/v) polysorbate-20, at approximately 4° C. Potassium phosphate buffer was then added to neutralize the pH. Some lots were concentrated and diafiltered at approximately 4° C. against 150 mM sodium chloride, 25 mM potassium phosphate pH 7, using a 30 kDa NMWCO tangential flow ultrafiltration membrane.

### Final Filtration and Product Storage

Individual batches were then concentrated and diafiltered against 10 mM histidine in 150 mM sodium chloride, pH 7.0, with 0.015% (w/v) polysorbate 20, at 4° C. using a 300 kDa NMWCO tangential flow ultrafiltration membrane. Specifically, for ST-5, halfway through the diafiltration step, ST-5 conjugate was harvested and incubated with 50 mM sodium bicarbonate, pH 9.3 for 3 hours. The ST-5 solution was neutralized with 1.5 M potassium phosphate, pH 6.0 prior to completing diafiltration.

The individual retentate batches were 0.2-micron filtered (with 0.5-micron prefilter) then diluted with additional 10 mM histidine in 150 mM sodium chloride, pH 7.0 with 0.015% (w/v) polysorbate 20, dispensed into aliquots and frozen at ≤−60° C. Serotype specific conjugate details can be found as previously described (WO2011/100151, WO2019/139692 and WO2020/131763).

### Example 2: Formulation of Pneumococcal Conjugate Compositions

Individual pneumococcal polysaccharide-carrier protein conjugates prepared utilizing different chemistries as described in Example 1 were used for the formulation of a 1-, 21-, or 24-valent pneumococcal conjugate composition referred to as PCV1, PCV21 or PCV24, respectively.

The PCV1 formulation, to be added to the CLA-SNE or SNE or used as is, contained serotype 6B conjugated using reductive amination in an aprotic (DMSO) solvent, as described in Example 1, and formulated in 20 mM L-Histidine pH 5.8, 150 mM NaCl and 0.1% (w/v) PS-20 for a final concentration of 4 μg/mL (w/v) pneumococcal polysaccharide (PnPs) in the vaccine. The PCV1 vaccine formulation, prepared with APA and serotype 6B conjugated using reductive amination in an aprotic (DMSO) solvent, as described in Example 1, was formulated in 20 mM L-Histidine pH 5.8, 150 mM NaCl and 0.2% (w/v) PS-20 and 250 mg [Al⁺³]/mL in the form of APA for a final concentration of 4 μg/mL (w/v) pneumococcal polysaccharide (PnPs) in the vaccine.

The PCV21 formulation, to be added to the CLA-SNE or SNE or used as is, contained serotypes 3, 6A, 7F, 8, 9N, 10A, 11A, 12F, 15A, deO-Acetylated-15B (deOAc15B), 16F, 17F, 19A, 20, 22F, 23A, 23B, 24F, 31, 33F and 35B conjugated to the carrier protein CRM197 using reductive amination in an aprotic solvent (e.g. DMSO) and formulated in 20 mM L-Histidine pH 5.8, 50-150 mM NaCl and 0.02-0.1% PS-20. Each polysaccharide-carrier protein conjugate was formulated at 4-8 μg/mL (w/v) pneumococcal polysaccharide (PnPs) for a final concentration of 84-168 μg/mL PnPs in the vaccine.

The PCV24 formulation, to be added to the CLA-SNE or SNE or used as is, contained serotypes 1, 3, 4, 5, 6A, 6B, 7F, 8, 9V, 10A, 11A, 12F, 14, 15A, de-O-Ac-15B, 18C, 19A, 19F, 22F, 23B, 23F, 24F, 33F and 35B. conjugated to the carrier protein CRM197 using reductive amination in an aprotic solvent (e.g. DMSO) and formulated in 20 mM L-Histidine pH 5.8, 50-150 mM NaCl and 0.02-0.1% PS-20. Each polysaccharide-carrier protein conjugate was formulated at 4 μg/mL (w/v) pneumococcal polysaccharide (PnPs) for a final concentration of 96 μg/mL PnPs in the vaccine.

To prepare the PCV formulations, the required volumes of monovalent bulk conjugates needed to obtain the indicated final concentration of (w/v) pneumococcal polysaccharide (also referred to as PnPs) were calculated based on the batch volume and the bulk polysaccharide concentration.

The formulation process consisted of a conjugate bulk blend preparation at 2× the final concentration of PnPs blends in 20 mM Histidine, 0.05 to 0.15% (w/v) PS-20, and 150 mM sodium chloride, pH 5.8.

Histidine pH 5.8, PS-20 and sodium chloride solutions were prepared and added to the formulation vessel. The individual pneumococcal polysaccharide-carrier protein conjugates, stored frozen, were thawed at 2-8° C. and then added to the formulation vessel. During the addition of polysaccharide-carrier protein conjugate to the formulation buffer (conjugate blend), the vessel was mixed to ensure homogeneity using a magnetic sir bar or magnetic impeller. After all additions were made and the solution was stirred, the conjugate blend was passed through sterilizing filters and collected in a vessel with or without APA. In some cases, the sterilizing filters were chased with 150 mM sodium chloride to adjust the batch to target concentration.

The formulations were filled into plastic syringes, glass syringes, or vials.

The PCV13 formulation, a 13-valent pneumococcal conjugate vaccine used herein, was sourced from commercially available PREVNAR13@.

### Example 3: Preparation of a Stable Nanoemulsion (SNE) Adjuvant System with and without the Cationic Lipid, (13Z,16Z)—N, N-dimethyl-3-nonyldocosa-13,16-dien-1-amine)

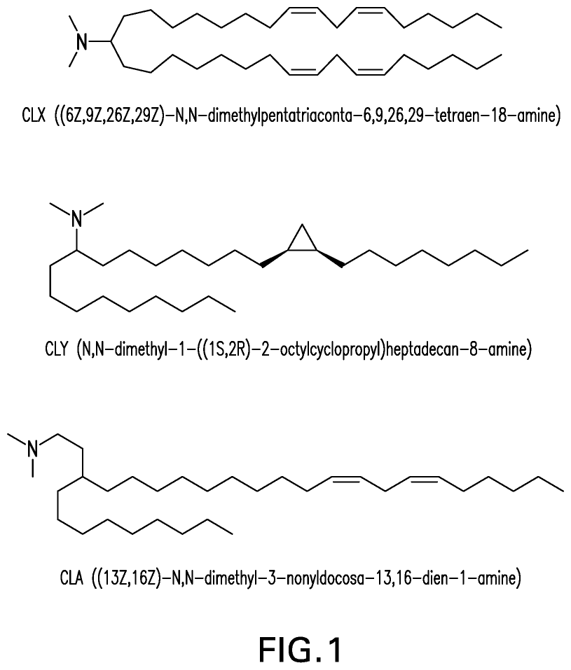

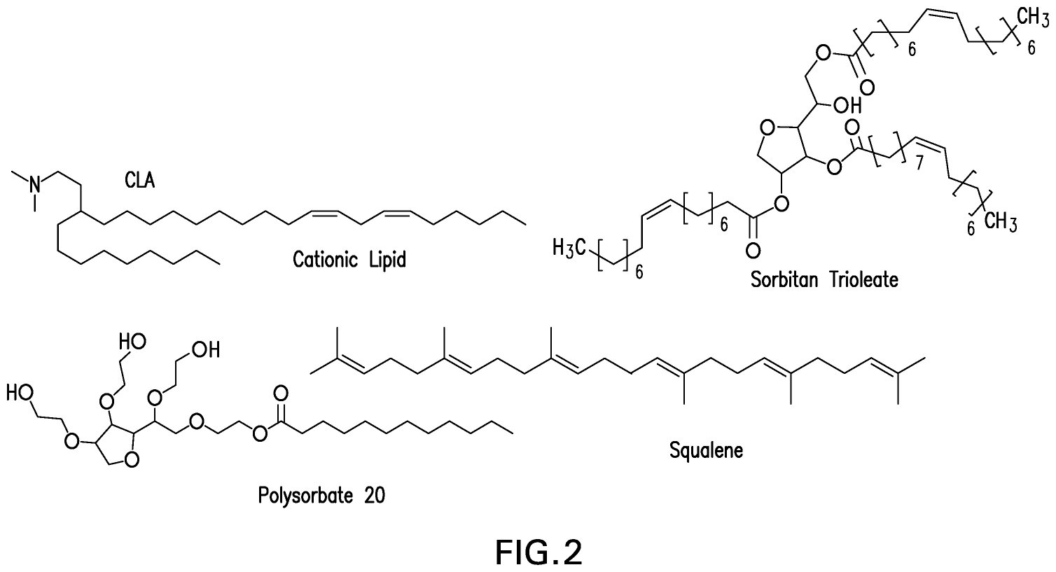

The SNE adjuvant can be prepared with and without cationic lipids, (13Z,16Z)—N,N-dimethyl-3-nonyldocosa-13,16-dien-1-amine) also referred to as CLA, or (6Z,9Z,26Z,29Z)—N,N-dimethylpentatriaconta-6,9,26,29-tetraen-18-amine, also referred to as CLX; or N,N-dimethyl-1-((1S,2R)-2-octylcyclopropyl)heptadecan-8-amine, also referred to as CLY (FIG. 1) The SNE is a multi-component emulsion formulation consisting of 3 stabilizing ingredients; squalene, sorbitan trioleate (SPAN-85), and polysorbate-20 (PS-20) with a cationic lipid, for example, CLA (referred to as CLA-SNE, see Table 1) and without a cationic lipid (referred to as SNE, see Table 2). This formulation was prepared by combining and mixing the cationic lipid (if used), squalene, SPAN-85 and PS-20 or similar (e.g. surfactants, oils, and solubilizers) components together (Table 1 and FIG. 2). Once mixed and blended, a histidine buffer was added and mixed with the initial emulsion components. Blended emulsion components were first subjected to course homogenization followed by fine homogenization, as described below. The resulting formulation was subjected to a final 0.2 μm filtration step. Several process parameters within each step, such as order of addition, mixing time, pH, temperature, concentration of components, homogenization, microfluidization were controlled to yield an emulsion system with desired attributes.

TABLE 1
Composition of a Representative CLA-SNE Adjuvant

| | | Content of | Molecular | Content |
|---|---|---|---|---|
| | | Each Lipid | Weight | of Each |
| Component | Description | (Mole %) | (g/mol) | Lipid (Mass %) |
| CLA | (13Z, 16Z)-N,N-dimethyl- | 13.82-44.5 | 475.9 | 14.29-45.45 |
| | 3-nonyldocosa-13,16- | | | |
| | dien-1-amine | | | |
| Squalene | squalene | 51.56-80.07 | 410.72 | 45.45-71.43 |
| SPAN-85 | sorbitan trioleate | 2.21-3.43 | 957.5 | 4.55-7.14 |
| PS-20 | polysorbate-20 | 1.72-2.68 | 1228 | 4.55-7.14 |
| Buffer | 20 mM Histidine, pH 5.8 | N/A | | |
| Matrix | | | | |

TABLE 2
Composition of a Representative SNE Adjuvant

| | | Content of | Molecular | Content |
|---|---|---|---|---|
| | | Each Lipid | Weight | of Each |
| Component | Description | (Mole %) | (g/mol) | Lipid (Mass %) |
| Squalene | squalene | 92.25-93.57 | 410.72 | 81.97-84.75 |
| SPAN-85 | sorbitan trioleate | 3.61-4.35 | 957.5 | 7.63-9.02 |
| PS-20 | polysorbate-20 | 2.82-3.39 | 1228 | 7.63-9.02 |
| Buffer | 20 mM Histidine, pH 5.8 | N/A | | |
| Matrix | | | | |

### Formulation Preparation

The squalene and solubilizer formulation (referred to as the oil phase) of the stable emulsion was prepared by addition of squalene, SPAN-85, PS-20 and CLA to a vessel. The oil phase was then mixed using magnetic stirring at 100-1000 RPM for 10 to 120 minutes. After mixing of these components, an aqueous phase comprised of 20 mM Histidine pH 5.8, was slowly added to the oil phase while being mixed using a magnetic stir bar. This formulation was then mixed again for 1 hour.

### Coarse Homogenization

The oil and aqueous phase mixture (referred to as the pre-homogenized emulsion or PHE) was then homogenized and size reduced to form a rough emulsion using a rotor stator homogenizer at ambient temperature. The homogenizer arm tip was submerged into the PHE and held in place near the bottom of the formulation vessel and was operated at 6 to 10 kRPM for 5-15 minutes. This process resulted in a homogenous micro-emulsion (ME) suspension of squalene emulsion particles in the 4 to 20 μm diameter range which were suitable for additional size reduction by microfluidization in a high-pressure homogenizer to create a stable nanoemulsion (SNE).

### Fine Homogenization to Produce the Stable Nanoemulsion (SNE)

After coarse homogenization, the emulsion was further processed using a high-pressure homogenizer/microfluidizer to produce stable nanometer-sized emulsion particles. The ME was introduced to a high-pressure homogenizer such as the Microfluidics low volume Microfluidizer®, the GEA Group PandaPlus 2000 or Bee International, NanoDeBEE and a recirculation loop is established. A counter-flow heat exchanger, fed by a Controlled Temperature Unit with a set point of 5° C., is included in the recirculation loop to neutralize the heat generated through high pressure homogenization. For the production of emulsion particles of desired size and processability, 20 kPSI was selected as the operating set point for this process step. The high-pressure homogenizer operates at a constant and unalterable flow rate through the established recirculating loop. Using this measured flow rate and the volume of ME to be processed, the theoretical time required for the entirety of the formulation to make a single pass through the recirculation loop was calculated. Given this calculated single pass time, the high-pressure homogenizer was usually operated until the desired pass count of at least 10 was reached, yielding either the SNE or CLA-SNE.

### Filtration

After formulation, the SNE or CLA-SNE was passed through a 0.8/0.2 μm PES filter. A flux of 42 LMH through the filter was selected given its optimal mass yield and particle stability through filtration.

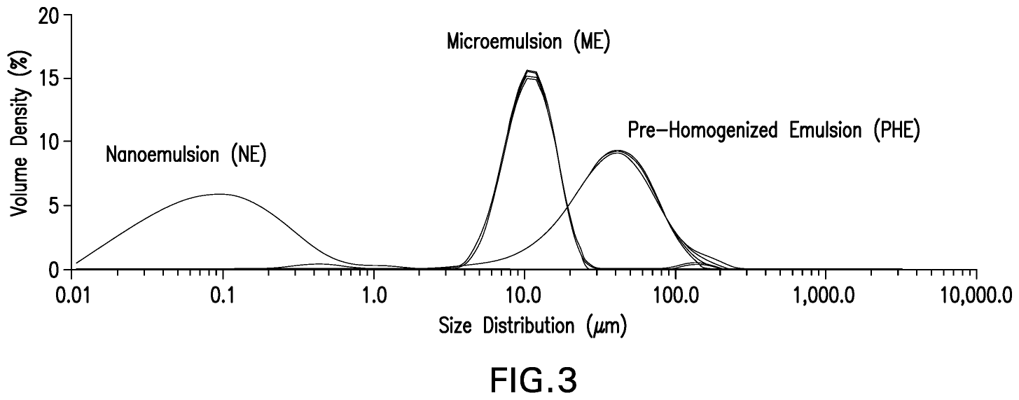

A laser diffraction or static light scattering (SLS) technique using a Malvern Panalytical Ltd. MS3000 instrument was utilized to measure the volume-weighed size distribution of a nanoemulsion during preparation. This data was then analyzed to calculate the size of the particles that created the scattering pattern. Sample fractions of pre-homogenized emulsion (PHE), micro-emulsion (ME), and stable nanoemulsion (SNE) were obtained. These emulsion formulations were diluted to target an obscuration of 3% into 5 mM Histidine pH 5.8 and 2.5 mM NaCl buffer and SLS was performed and collected under recirculation of 1200 RPM. Sample data sets were collected with a scan of 30 seconds per data set. Three data sets from each step in the CLA-SNE formulation process are summarized in FIG. 3. Although 20 mM Histidine pH 5.8 buffer was a perfectly suitable formulation for the stability of the bulk during process and when stored in polymeric containers (e.g. plastic), upon storage in glass, non-specific absorption of the CLA-SNE or SNE to the surface of the glass was observed. A screen evaluating surfactants/solubilizers, buffers and salts was evaluated, and multiple formulations show success in eliminating this stability issue with the selected formulation of 20 mM Histidine 0.05% PS-20 and 75 mM NaCl being selected as the stabilizing formulation (data not shown).

### Example 4: Preparation of a Stable Nanoemulsion (SNE) Adjuvant System and Addition of the Cationic Lipid, (13Z,16Z)—N, N-dimethyl-3-nonyldocosa-13,16-dien-1-amine) or CLA as Free Base Directly after Microfluidization of the SNE

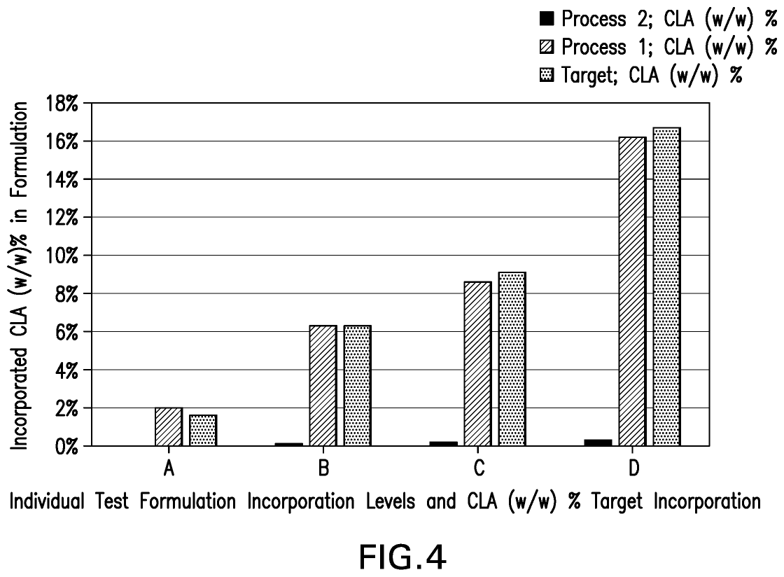

Two formulation processes were evaluated for incorporating CLA into the nanoemulsion particle which includes PS-20, sorbitan trioleate (SPAN-85) and squalene formulated in Histidine pH 5.8 buffer. In the first process (referred to as Process 1), the SNE was prepared using the process described in Example 3. In the second process, (referred to as Process 2), only PS-20, sorbitan trioleate (SPAN-85) and squalene were combined and mixed together. Once mixed and blended, a histidine buffer was added and mixed with the initial emulsion components (PS-20, sorbitan trioleate and squalene). Blended emulsion components were first subjected to course homogenization to produce the microemulsion ME followed by microfluidization to produce the nanoemulsion (NE), as described in Example 3. In a separate glass vessel, 0.25 mg/mL CLA was dissolved in 100% ethanol at room temperature. A sufficient volume of this CLA ethanol solution, to produce the desired final CLA concentration, was then added to the SNE containing PS-20, sorbitan trioleate and squalene in Histidine buffer pH 5.8 and then mixed for 60 minutes at room temperature. After incubation, the formulation was then dialyzed against 5 mM histidine 2.5 mM NaCl pH 5.8 at 10 mL sample to 500 mL buffer over night at 4° C., with two buffer changes. The two processed emulsions (Process 1 and 2) were then examined for CLA incorporation into the SNE using UPLC-CAD. The results indicate that no significant quantity of CLA was incorporated as compared to the individual formulation (w/w) % target incorporation using Process 2 which indicates that Process 1 is preferred for the successful incorporation and stability of CLA in the nanoemulsion (FIG. 4).

### Example 5: Preparation of the Cationic Lipid Containing LNP Adjuvant

CLA-LNP is a multi-component LNP formulation consisting of 4 components; one cationic lipid (referred to as CLA; FIG. 1, shown with preferred Cationic Lipid, CLA, (13Z,16Z)—N,N-dimethyl-3-nonyldocosa-13,16-dien-1-amine), cholesterol, distearoyl phosphatidyl choline (DSPC), and ePEG-DMG.

The final CLA-LNP formulation relative target mole % values for the lipid components are 58% CLA, 30% cholesterol, 2% ePEG2000-DMG and 10% DSPC (Table 3).

TABLE 3
Composition of the CLA-LNP Adjuvant

| | | Content | | |
|---|---|---|---|---|
| | | of Each | | |
| | | Lipid | Molecular | Content of |
| | | (Mole | Weight | Each Lipid |
| Component | Description | %) | (g/mol) | (Mass %) |
| CLA | (13Z,16Z)-N,N-dimethyl-3- | 58 | 475.9 | 52 |
| | nonyldocosa-13, 16-dien-1-amine) | | | |
| Cholesterol | Cholest-5-en-3β-ol | 30 | 386.7 | 22 |
| Distearoyl | 1,2-distearoyl-sn-glycero-3- | 10 | 790.2 | 15 |
| Phosphitidyl | phosphocholine | | | |
| Choline (DSPC) | | | | |
| ePEG2000- | α-[8′-(1,2-Dimyristoyl-3-propanoxy)- | 2 | 2837 | 11 |
| DMG | carboxamide-3′, 6′- | | | |
| | Dioxaoctanyl]carbamoyl-ω-methyl- | | | |
| | poly(ethylene glycol)-2000 | | | |

| | | |
|---|---|---|
| Buffer Matrix | 20 mM Tris 10% (w/v) Sucrose pH 7.5) | N/A |

The process of making the CLA-LNP consists of 5 steps: 1) solution preparation of lipid mixture and diluted citrate A; 2) LNP formation by means of T-mixing; 3) ultra-filtration; 4) bioburden reduced filtration; and 5) sterile filtration and vial filling.

### Solution Preparation of Lipid Mixture and Diluted Citrate A

The lipid components were weighed and combined before being dissolved in ethanol before being sterile filtered to form the lipid mixture. Citrate A (20 mM Citrate pH 5.0) was diluted at a one ratio with sterile water to form diluted citrate A (DCA).

### LNP Formation by Means of T-Mixing

The lipid mixture and DCA were then mixed together at adjacent ends of a T-tube mixer. The stream exiting the T-mix apparatus was immediately diluted 1:1 with 20 mM Citrate, 300 mM NaCl pH 6.0, then this product mixture was again diluted 1:1 with 1× Dulbecco's phosphate buffered saline, and then collected as formed LNP. The LNP intermediate was then incubated at ambient temperature for 30 minutes before being held overnight at 4° C.

### Ultra-Filtration

The LNP intermediate was then subjected to ultra-filtration with a 500 kDa NMWCO in order to both concentrate the material approximately 10-fold as well as diafilter the material against 20 mM Tris, 10% (w/v) sucrose, pH 7.5. After the diafiltration, there was a final concentration step performed in order to achieve final target concentration.

### Bioburden Reduced Filtration

The adjuvant bulk was then pre-filtered with a 0.45 μm cellulose acetate (CA) filter followed by a 0.2 m CA bioburden-reducing filter, and stored frozen at −70° C.

### Sterile Filtration and Vial Filling

Frozen adjuvant bulk was thawed in a 25+3° C. controlled water bath. The thawed adjuvant bulk was passed through a 0.45 μm polyvinylidene fluoride (PVDF) bioburden reducing filter and a 0.22 μm PVDF sterilizing grad filter and received. The filtered adjuvant bulk was then diluted with 20 mM Tris, 10% (w/v) sucrose, pH 7.5 to the target LNP adjuvant concentration. This diluted final bulk adjuvant was then filled into glass vials and stored at −70° C.

### Example 6: PCV1 Immunogenicity in Mice: Evaluation of Adjuvant Systems

Young female BALB/c mice (6-8 weeks old, n=10/group) were intramuscularly (IM) immunized with 0.1 mL of PCV1 formulated with different adjuvants (Table 4) on days 0, day 14, and day 28. PCV1 was dosed at 0.08 μg PnPs (6B conjugated to CRM197) per immunization. Mice were observed at least daily by trained animal care staff for any signs of illness or distress. The vaccine formulations in mice were deemed to be safe and well tolerated, as no vaccine-related adverse events were noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc. (Kenilworth, NJ, USA).

TABLE 4
Formulation Compositions assessed in
PCV1 Immunogenicity Study in Mice
Formulation
ST-6B-CRM197; 0.8 μg PnPs/mL; 20 mM L-histidine,
150 mM NaCl, 0.1% w/v PS-20 [No Adjuvant]
ST-6B-CRM197; 0.8 μg PnPs/mL 20 mM L-histidine,
150 mM NaCl, 0.2% w/v PS-20; [50 μg/mL APA]
ST-6B-CRM197; 0.8 μg PnPs/mL; 10 mM L-histidine,
75 mM NaCl, 0.05% w/v PS-20 [8 μg/mL CLA-SNE]
ST-6B-CRM197; 0.8 μg PnPs/mL; 10 mM L-histidine,
75 mM NaCl, 0.05% w/v PS-20 [80 μg/mL CLA-SNE]
ST-6B-CRM197; 0.8 μg PnPs/mL; 10 mM L-histidine,
75 mM NaCl, 0.05% w/v PS-20 [800 μg/mL CLA-SNE]
ST-6B-CRM197; 0.8 μg PnPs/mL; 10 mM L-histidine,
75 mM NaCl, 0.05% w/v PS-20
[SNE (10 mg/mL of squalene; 1.0 mg/mL of PS-20;
1.0 mg/mL of SPAN-85)]
10 mM L-histidine, 75 mM NaCl, 0.05% w/v PS-20
[SNE (10 mg/mL of squalene; 1.0 mg/mL of PS-20;
1.0 mg/mL of SPAN-85)]

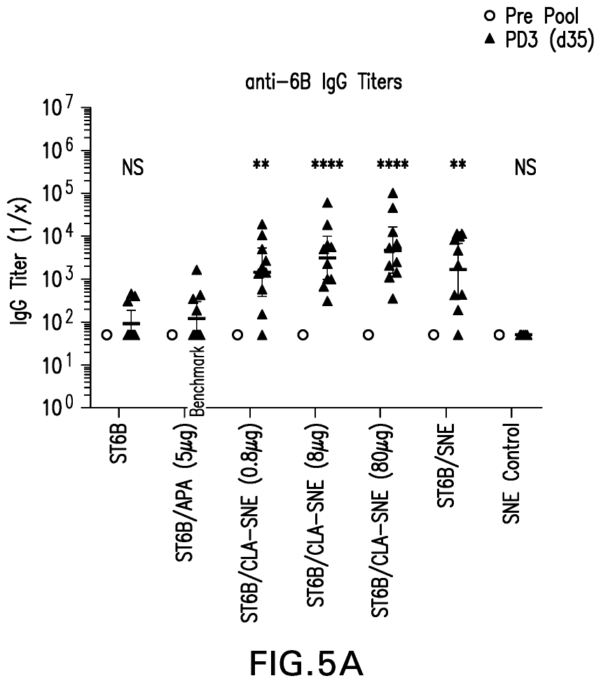

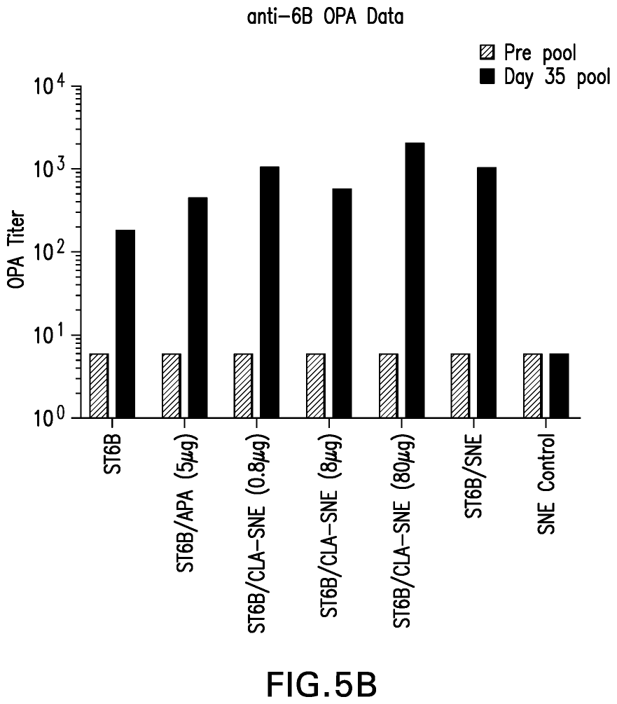

Mouse sera were evaluated for IgG immunogenicity using ELISA to assess anti-6B IgG titers. Functional antibody was determined through opsonophagocytic assays (OPA) based on previously described protocols available from the UAB Pneumococcal Reference Laboratory (University of Alabama Reference Laboratory at Birmingham Bacterial Respiratory Pathogen Reference Library) and Opsotiter® 3 software owned by and licensed from University of Alabama (UAB) Research Foundation (See, Caro-Aguilar I. et al., Vaccine (2017) 35(6):865-72 and Burton R. L. and Nahm M. H. Clin. Vaccine Immunol. (2006) 13(9):1004-9). As shown in FIG. 5A, PCV1 immunization generated antibody titers in BALB/c mice for the 6B serotype (ST-6B) in the vaccine when formulated with and without APA. ST-6B, formulated with CLA-SNE (at either 0.08, 8 or 80 mg CLA per dose) or SNE adjuvants, were found to be immunogenic in mice and resulted in higher immunogenicity at post dose 3 as compared to ST-6B formulated alone or with APA. Anti-6B functional antibody titers were generated in BALB/c mice that were immunized with ST-6B formulated with and without APA (FIG. 5B).

### Example 7: PCV24 Immunogenicity in Adult Rhesus Macaques

PCV24 (Serotypes-1, 3, 4, 5, 6A, 6B, 7F, 8, 9V, 10A, 11A, 12F, 14, 15A, de-O-Ac-15B, 18C, 19A, 19F, 22F, 23B, 23F, 24F, 33F and 35B each individually conjugated to CRM197) was assessed in an adult rhesus macaque immunogenicity model. Rhesus macaques were intramuscularly immunized with PCV24 formulated with APA or PCV24 formulated with SNE or either CLA formulated as an SNE (CLA-SNE) or LNP (CLA-LNP) (Table 5) on days 0, and 28. PCV24 was dosed at 0.4 μg PnPs in a 0.1 mL volume per immunization. Sera were collected prior to study start (pre-immune, day 0) and on days 14 (PD1) and 42 (PD2).

TABLE 5
PCV24 Formulations Evaluated in an Adult
Rhesus Macaque Immunogenicity Model
Formulation
PCV24 in 20 mM L-histidine, 150 mM NaCl,
0.2% w/v PS-20 [250 μg/mL APA]
PCV24 in 10 mM L-histidine, 10 mM Tris, 5% (w/v) sucrose,
75 mM NaCl, 0.05% w/v PS-20 [1200 μg/mL CLA-LNP]
PCV24 in 10 mM L-histidine, 75 mM NaCl, 0.05% w/v PS-20
[800 μg/mL CLA-SNE (25 mg/mL of squalene; 5.0 mg/mL
of PS-20; 5.0 mg/mL of SPAN-85)]
PCV24 in 10 mM L-histidine, 75 mM NaCl, 0.05% w/v PS-20
[1200 μg/mL CLA-SNE (25 mg/mL of squalene; 5.0 mg/mL
of PS-20; 5.0 mg/mL of SPAN-85)]
PCV24 in 10 mM L-histidine, 75 mM NaCl, 0.05% w/v PS-20
[SNE (25 mg/ml of squalene; 5.0 mg/mL of PS-20;
5.0 mg/mL of SPAN-85)]

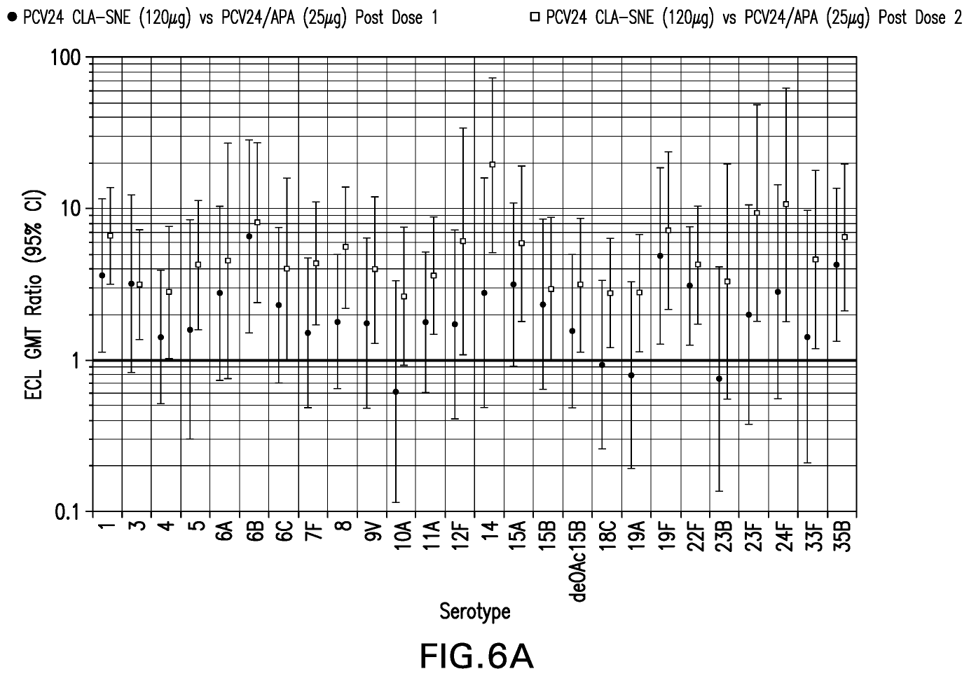

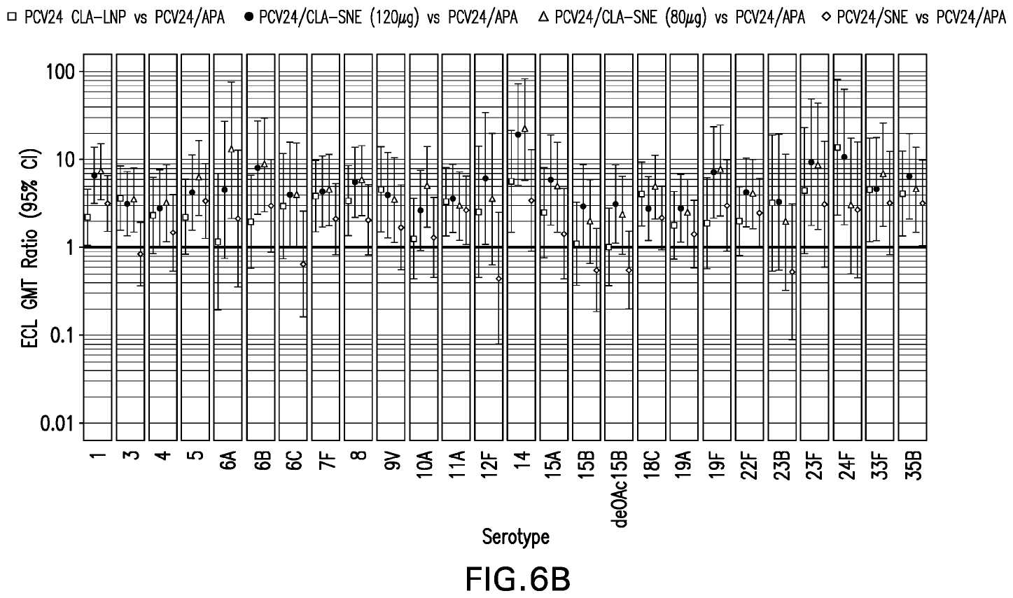

To assess serotype-specific IgG immunogenicity responses in a 24-valent vaccine, a multiplexed electrochemiluminescence (ECL) assay was developed. This assay was developed for use with rhesus serum based on previous assays described by Marchese et al. and Skinner et al. (Marchese R. D. et al., Clin. Vaccine Immunol. (2009) 16(3):387-96 and Skinner, J. M. et al., Vaccine (2011) 29(48):8870-8876). Technology developed by MesoScale Discovery (a division of MesoScale Diagnostics, LLC, Gaithersburg, MD) utilizes pneumococcal serotype polysaccharides (1, 3, 4, 5, 6A, 6B, 6C, 7F, 8, 9V, 10A, 11A, 12F, 14, 15A, 15B, de-O-Ac-15B, 18C, 19A, 19F, 22F, 23B, 23F, 24F, 33F, 35B) coated on spots in a 96-well plate and a SULFO-TAG™ labeled antibody that emits light upon electrochemical stimulation. SULFO-TAG™-labeled anti-human IgG was used as the secondary antibody for testing rhesus serum samples. Endpoint dilution titer was calculated as the reciprocal of the linearly interpolated dilution corresponding to the cutoff value (ECL signal of control) using logarithmic scaling for the ECL and the dilution. Titers were extrapolated for samples beyond the studied maximum dilution, based on linear extrapolation (in the log-log scaling) using the intercept and slope of the last 2 or 3 ECL assay data points for the sample curve completely above the cutoff line. All titers were obtained by back-transforming the linearly extrapolated dilution. If the sample curve was completely below the cutoff line, 100 was used as the titer in all data analysis and in FIGS. 6A and 6B. Functional antibody was determined through multiplexed opsonophagocytic assays (MOPA) based on previously described protocols available from the UAB Pneumococcal Reference Laboratory (University of Alabama Reference Laboratory at Birmingham Bacterial Respiratory Pathogen Reference Library) and Opsotiter® 3 software owned by and licensed from University of Alabama (UAB) Research Foundation (See, Caro-Aguilar I. et al., Vaccine (2017) 35(6):865-72 and Burton R. L. and Nahm M. H. Clin. Vaccine Immunol. (2006) 13(9):1004-9).

PCV24 formulated with the CLA-SNE formulated at 1200 μg/mL CLA-SNE with (25 mg/mL of squalene; 5.0 mg/mL of PS-20; 5.0 mg/mL of SPAN-85) was found to be immunogenic in adult rhesus macaques and resulted in higher immunogenicity at post dose 1 and post dose 2 as compared to PCV24 formulated with APA (FIG. 6A). PCV24 formulated with CLA-SNE at a dose level of 120 μg results in equal or better immunogenicity as compared to PCV24 formulated with APA (solid line) for post dose 1 (PD1) and post dose 2 (PD2). At PD2, PCV24 formulated with CLA-SNE at two dose levels of CLA (120 μg shown as circles or 80 μg shown as triangles) results in equal or better immunogenicity as compared to PCV24 formulated with APA (solid line) or PCV24 formulated with CLA-LNP (120 μg as squares) (FIG. 6B).

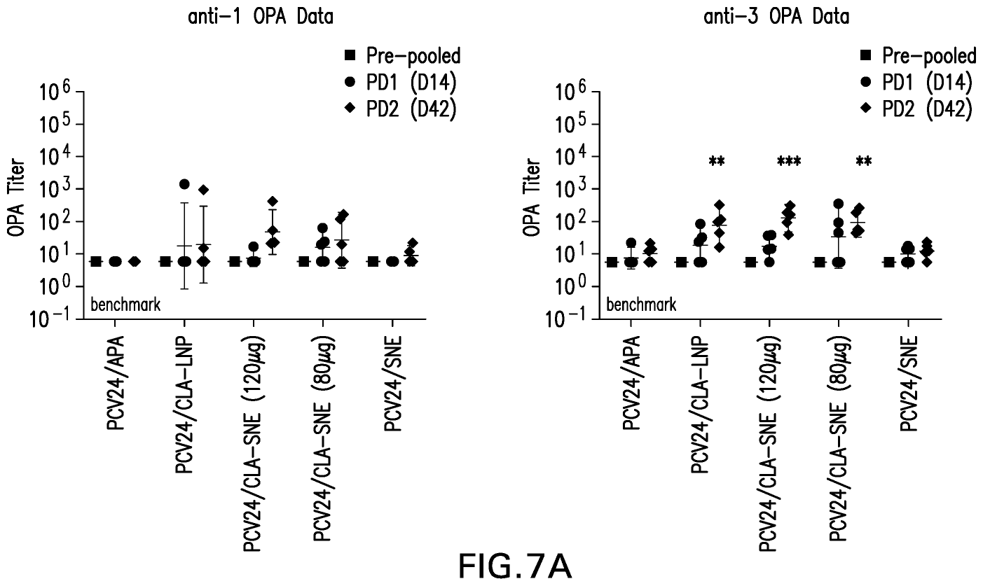

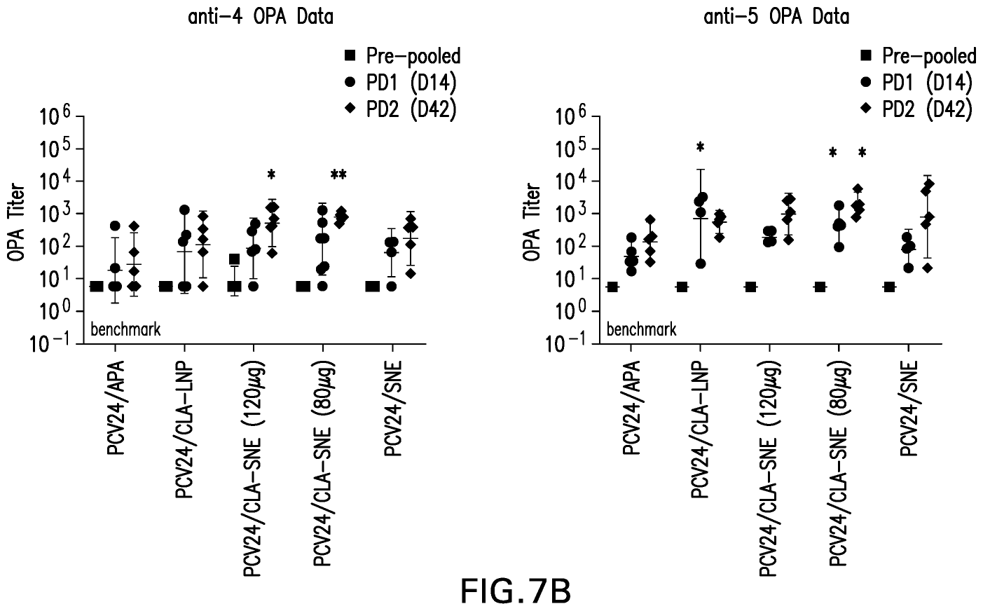

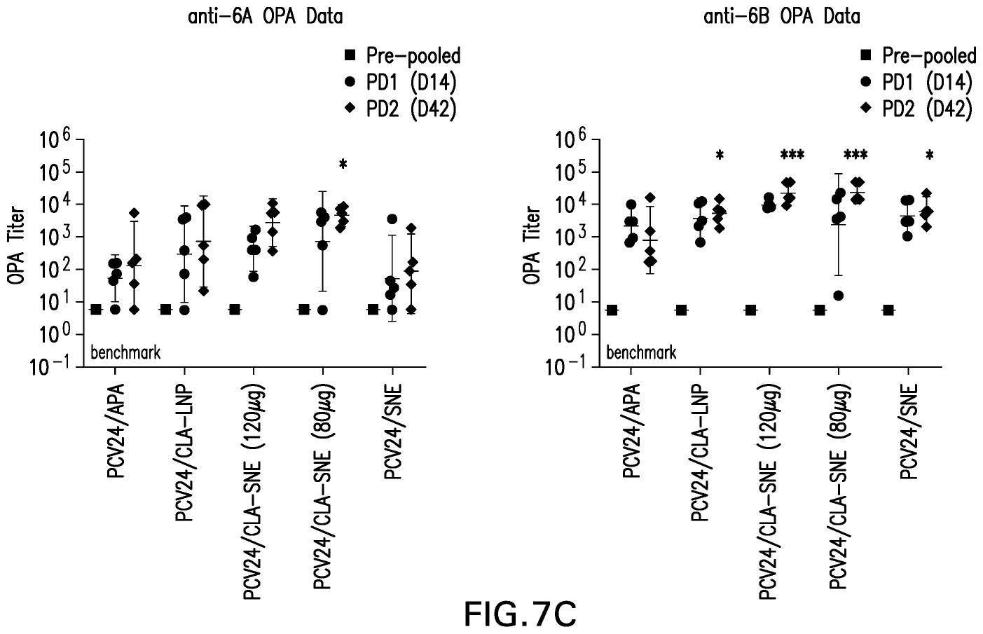

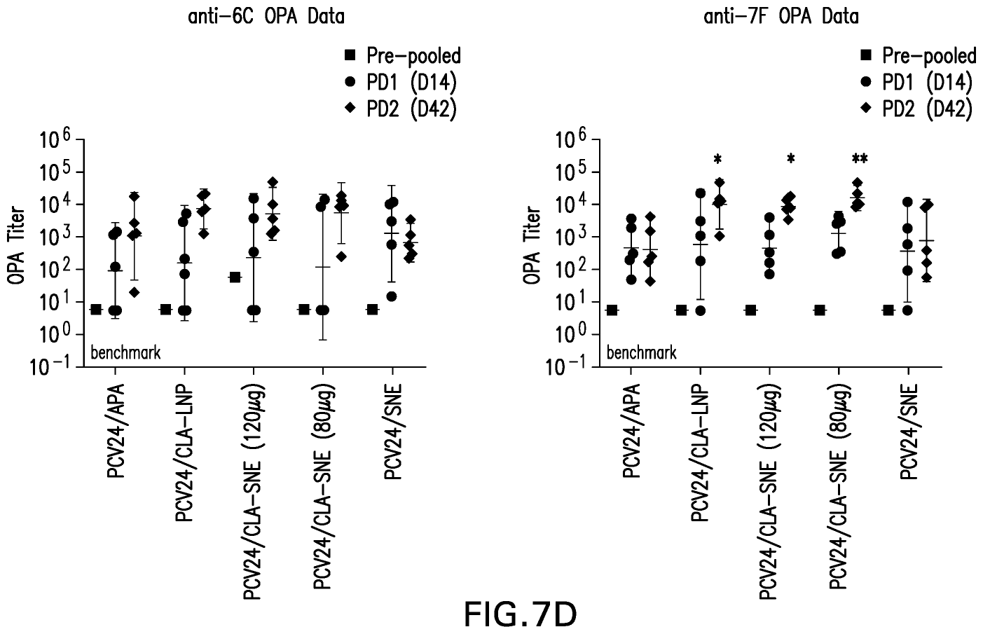

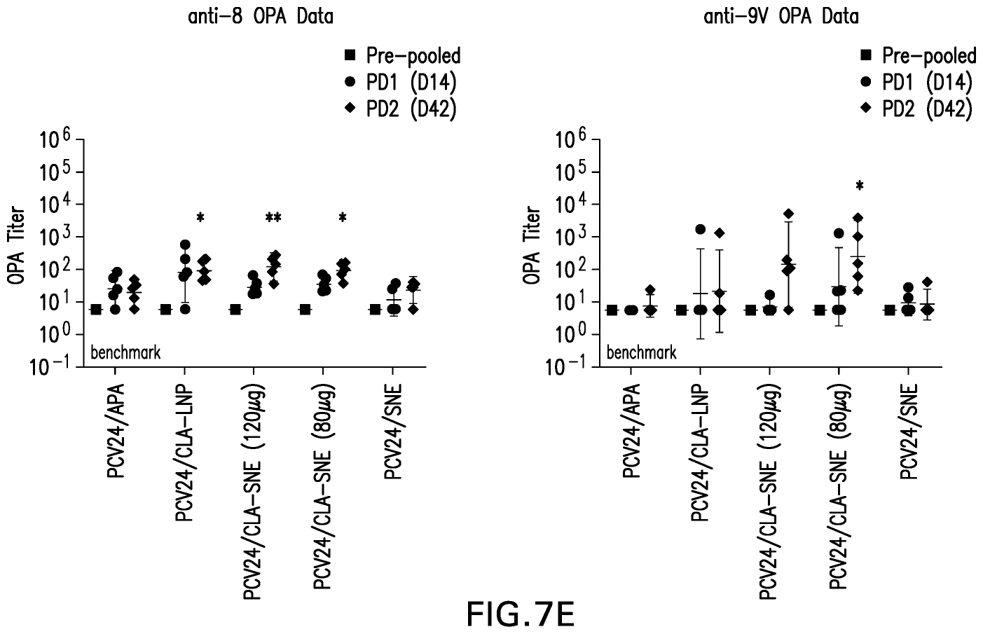

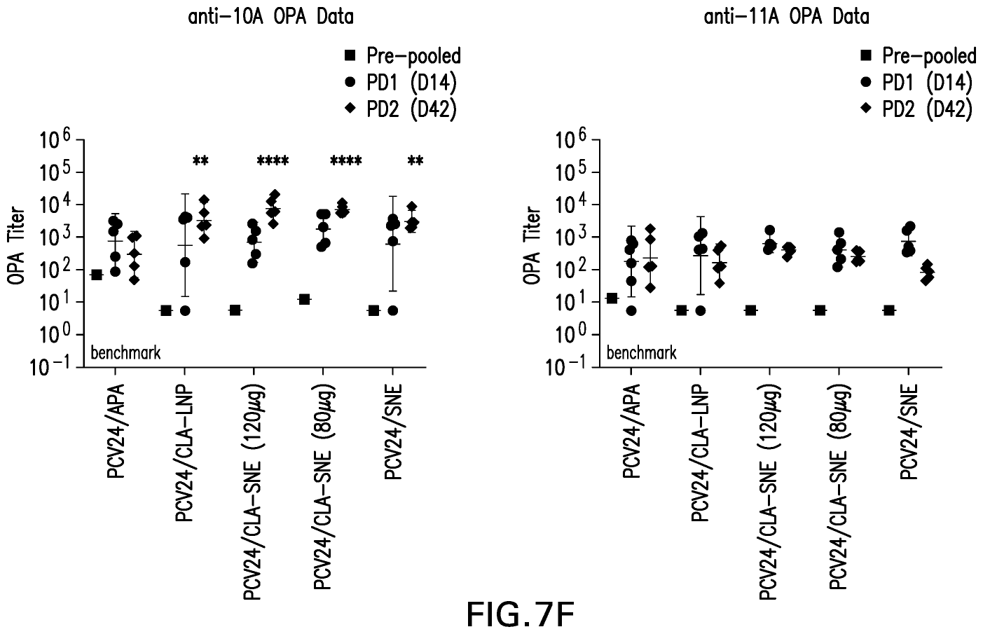

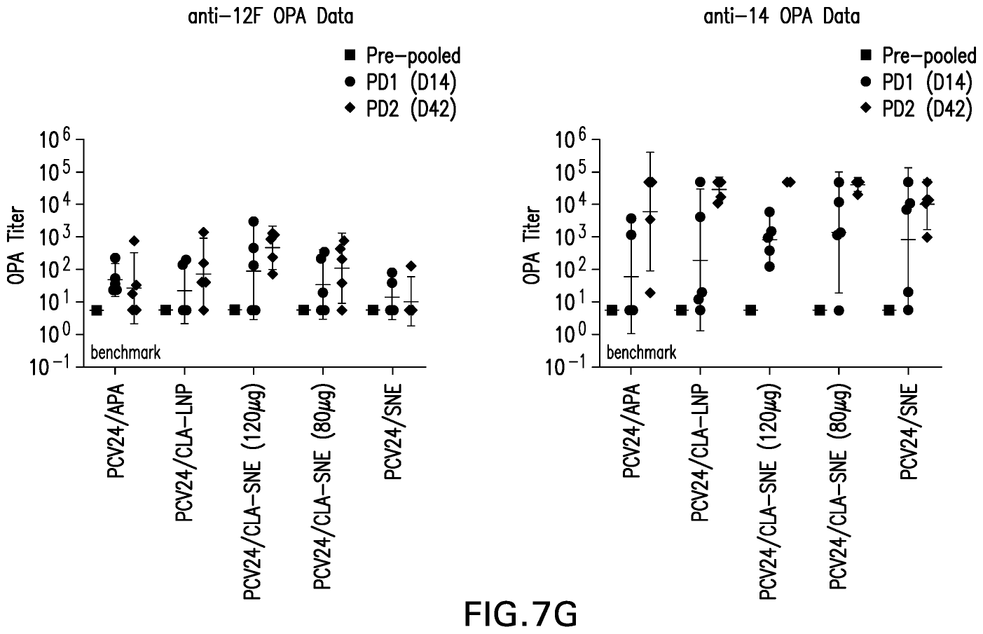

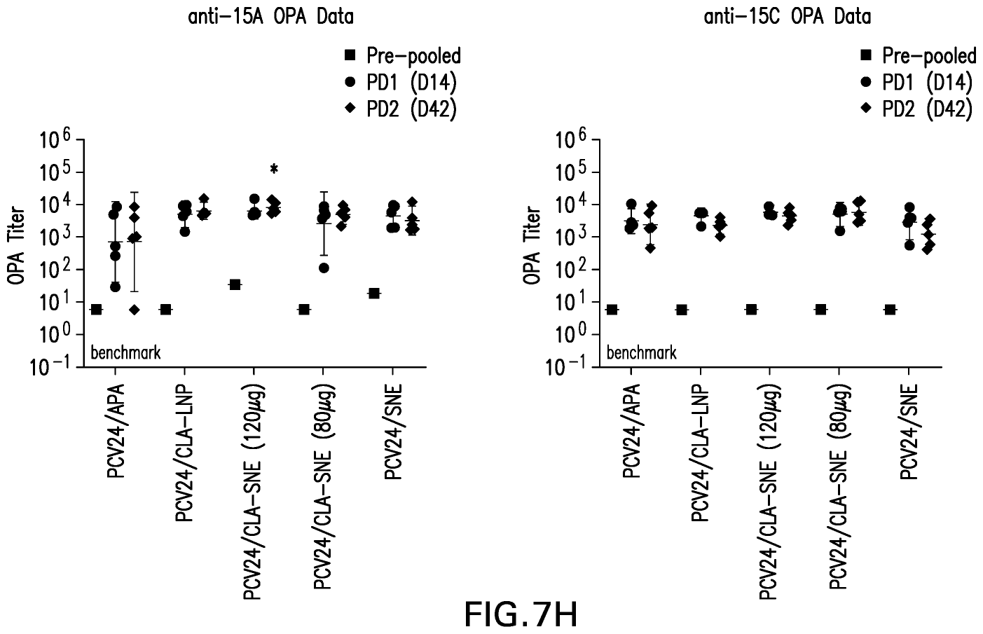

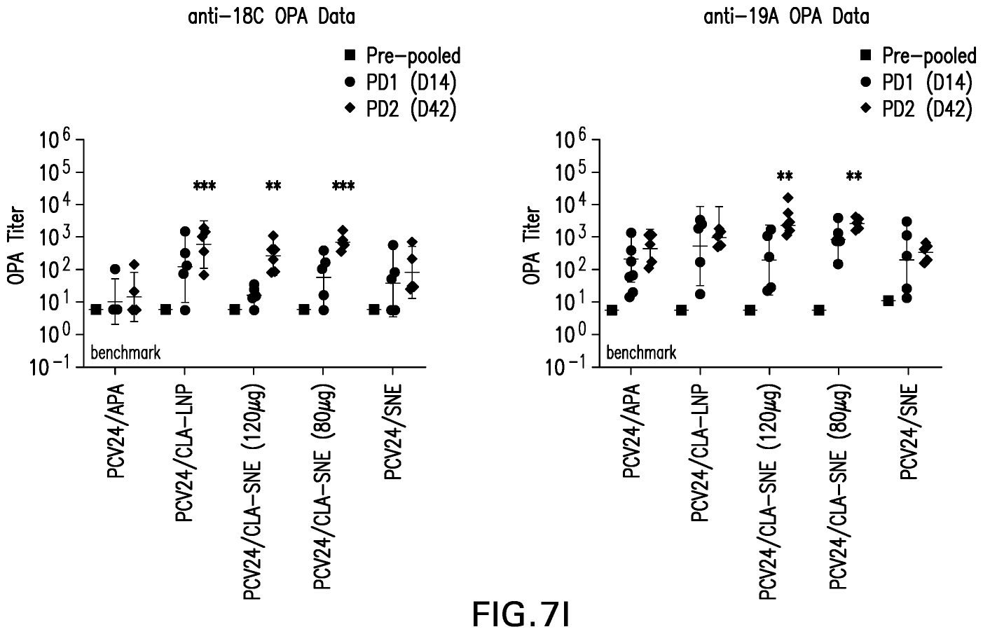

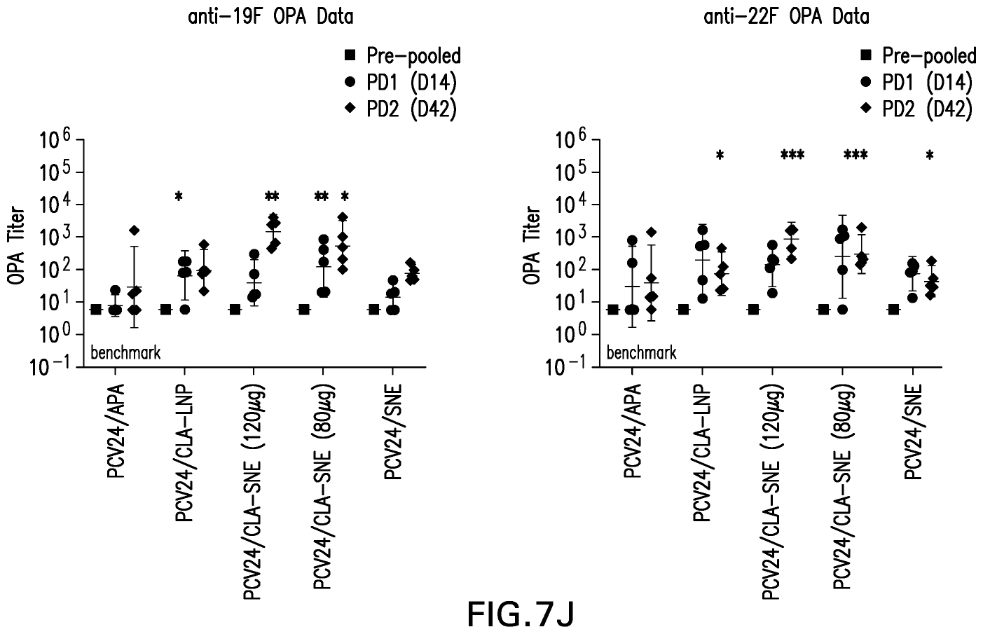

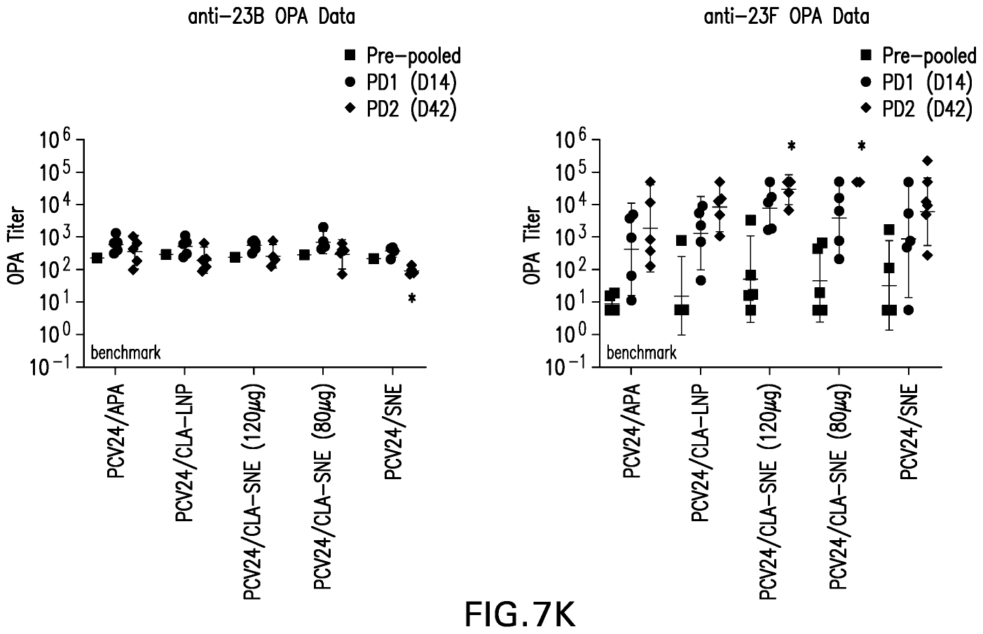

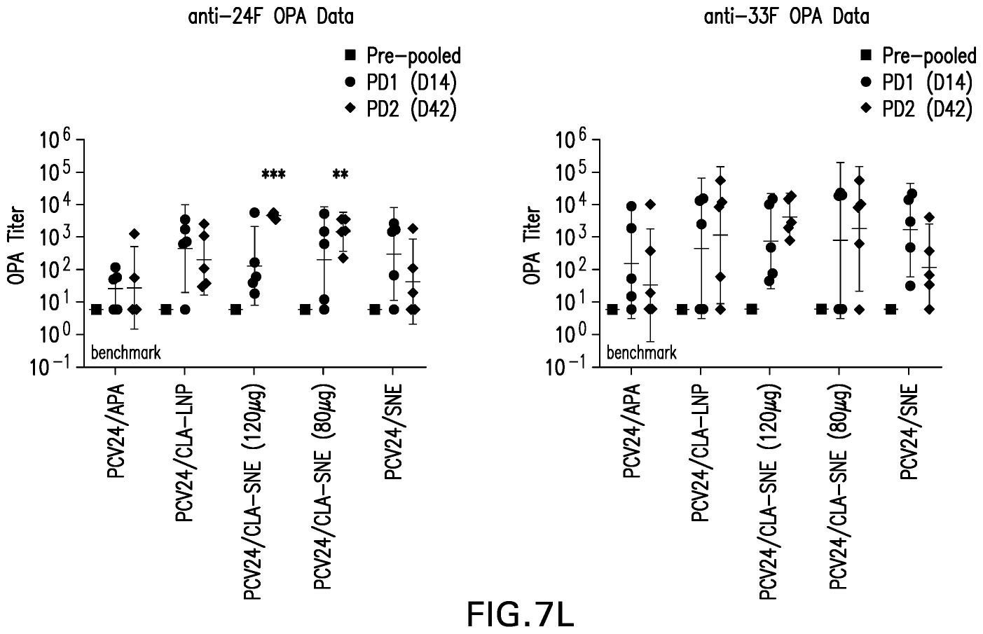

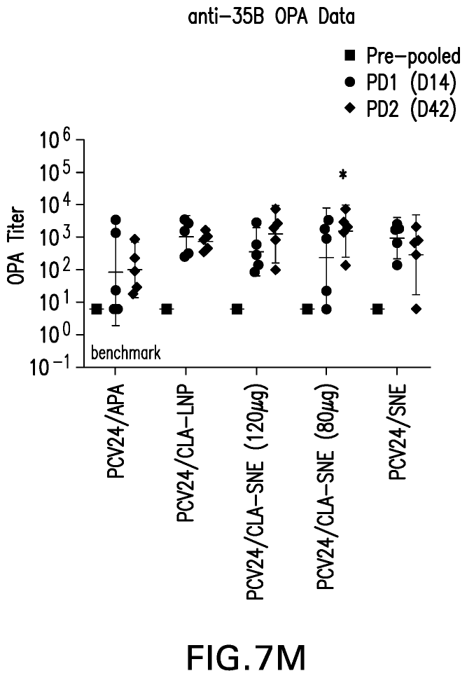

These same formulations generated functional antibodies (FIGS. 7A-7M) which killed vaccine-type bacterial strains at all time points tested and again the CLA-SNE, SNE alone and CLA-LNP boosted PCV24 response as compared to PCV24 formulated with APA. One exception was 23B whose high pneumococcal preimmunization interfered with a boost response following vaccination. All study time points were tested in MOPA as pooled or individual samples (FIGS. 7A-7M). Not much difference was observed in the OPA titers between PD1 and PD2.

PCV24 immunized adult rhesus macaque sera were evaluated for cross reactivity to other Streptococcus pneumoniae bacteria. PCV24 immunized macaque sera had cross reactivity with serotypes 6C (FIGS. 6A, 6B and 7D) and 15B (FIGS. 6A and 6B). The cross reactivity to 6C is likely due to immunization with polysaccharide conjugate 6A-CRM197 as part of a multivalent PCV24 (Cooper D, Yu X, Sidhu M, Nahm M H, Fernsten P, Jansen K U). The 13-valent pneumococcal conjugate vaccine (PCV13) elicits cross-functional opsonophagocytic killing responses in humans to Streptococcus pneumoniae serotypes 6C and 7A. Vaccine. 2011; 29:7207-11). Similarly, immunization with polysaccharide conjugates de-O-Ac-15B-CRM197 as part of a multivalent PCV resulted in cross reactivity to serotype 15C (Rajam et a1., Clinical and Vaccine Immunology, 2007, 14(9):1223-1227).

### Example 8: PCV24 Immunogenicity Study in Infant Rhesus Monkey (IRM)

PCV24 (serotypes-1, 3, 4, 5, 6A, 6B, 7F, 8, 9V, 10A, 11A, 12F, 14, 15A, de-O-Ac-15B, 18C, 19A, 19F, 22F, 23B, 23F, 24F, 33F and 35B each individually conjugated to CRM197) and adjuvant formulations were prepared as described in Examples, supra. IRMs (Infant Rhesus Monkeys, n=5/group) were intramuscularly immunized with 0.1 mL vaccine, as described in Table 6, below, on days 0, 28 and 56. Sera were collected prior to study start (pre) and on days 14 (PD1), 42 (PD2), and 70 (PD3). IRMs were observed twice daily by trained animal care staff for any signs of illness or distress. The vaccine formulations in IRMs were deemed to be safe and well tolerated, as no vaccine-related adverse events were noted.

TABLE 6
PCV24 Formulations Evaluated in an Infant
Rhesus Macaque Immunogenicity Model
Formulation
PCV24 in 20 mM L-histidine, 150 mM NaCl, 0.2% w/v PS-20,
[250 μg/mL APA]
PCV24 in 10 mM L-histidine, 10 mM Tris, 5% (w/v) sucrose,
75 mM NaCl, 0.05% w/v PS-20, [1200 μg/mL CLA-LNP]
PCV24 in 10 mM L-histidine, 75 mM NaCl, 0.05% w/v PS-20,
[SNE (25 mg/mL of squalene; 2.5 mg/mL of PS-20;
2.5 mg/mL of SPAN-85)]
PCV24 in 10 mM L-histidine, 75 mM NaCl, 0.05% w/v PS-20,
[600 μg/mL CLA-SNE (25 mg/mL of squalene;
2.5 mg/mL of PS-20; 2.5 mg/mL of SPAN-85)]
PCV24 in 10 mM L-histidine, 75 mM NaCl, 0.05% w/v PS-20,
[1200 μg/mL CLA-SNE (25 mg/mL of squalene;
2.5 mg/mL of PS-20; 2.5 mg/mL of SPAN-85)]
PCV24 in 10 mM L-histidine, 10 mM Tris, 5% sucrose,
75 mM NaCl, 0.05% w/v PS-20,
[2950 μg/mL CLA-SNE (25 mg/mL of squalene;
2.5 mg/mL of PS-20; 2.5 mg/mL of SPAN-85)]
PCV24 in 10 mM L-histidine, 75 mM NaCl, 0.05% w/v PS-20,
[600 μg/mL CLA-SNE (5 mg/mL of squalene;
0.5 mg/mL of PS-20; 0.5 mg/mL of SPAN-85)]
PCV24 in 10 mM L-histidine, 75 mM NaCl, 0.05% w/v PS-20,
[1200 μg/mL CLA-SNE (5 mg/ml of squalene;
0.5 mg/ml of PS-20; 0.5 mg/mL of SPAN-85)]
PCV24 in 10 mM L-histidine, 75 mM NaCl, 0.05% w/v PS-20,
[2950 μg/mL CLA-SNE (5 mg/mL of squalene;
0.5 mg/mL of PS-20; 0.5 mg/mL of SPAN-85)]
[PCV24 in 20 mM L-histidine, 75 mM NaCl, 0.05% w/v PS-20,
[1200 μg/mL CLA-SNE (0.8 mg/mL of squalene;
0.08 mg/mL of PS-20; 0.08 mg/mL of SPAN-85)]

To assess serotype-specific IgG responses in a 24-valent vaccine, a multiplexed electrochemiluminescence (ECL) assay was developed for use as described above. Endpoint titer was calculated as the reciprocal of the linearly interpolated dilution corresponding to the cutoff value (ECL signal of control) using logarithmic scaling for the ECL and the dilution. Titers were extrapolated for samples beyond the studied maximum dilution, based on linear extrapolation (in the log-log scaling) using the intercept and slope of the last 2 or 3 ECL assay data points for the sample curve completely above the cutoff line. Titers were then obtained by back-transforming the linearly extrapolated dilution. If the sample curve was completely below the cutoff line, 100 was used as the titer in all data analysis and in Figures.

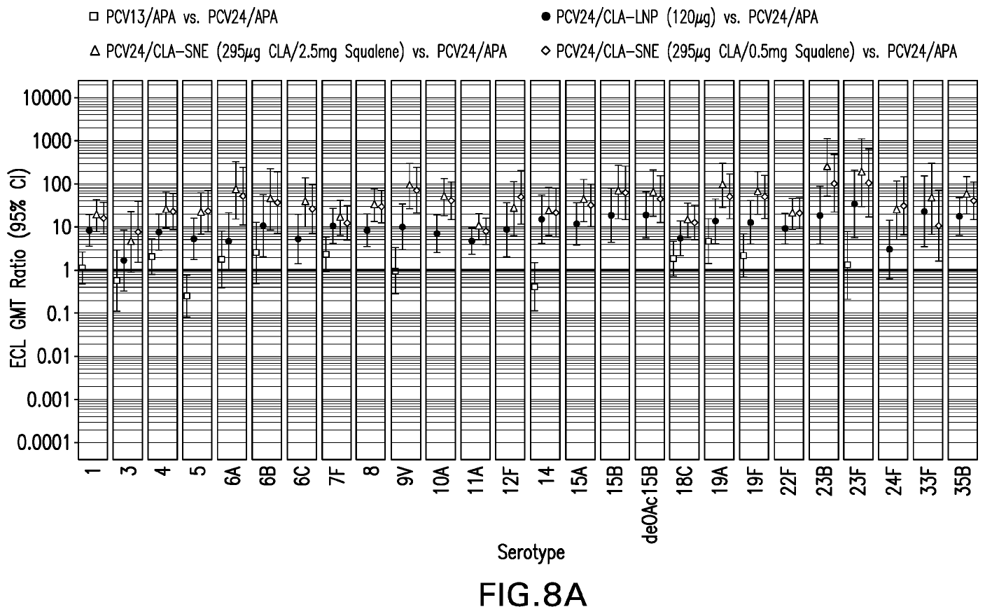

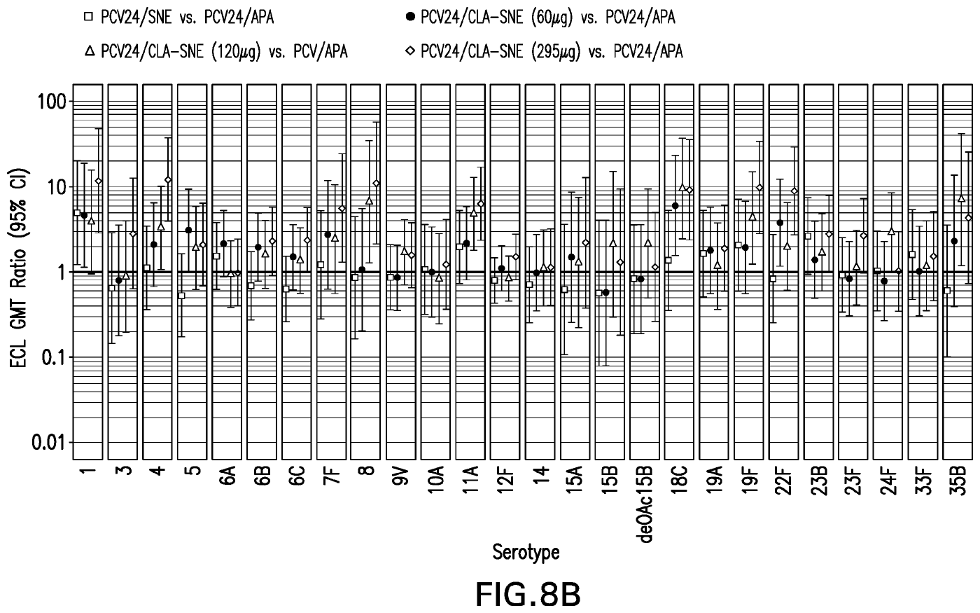

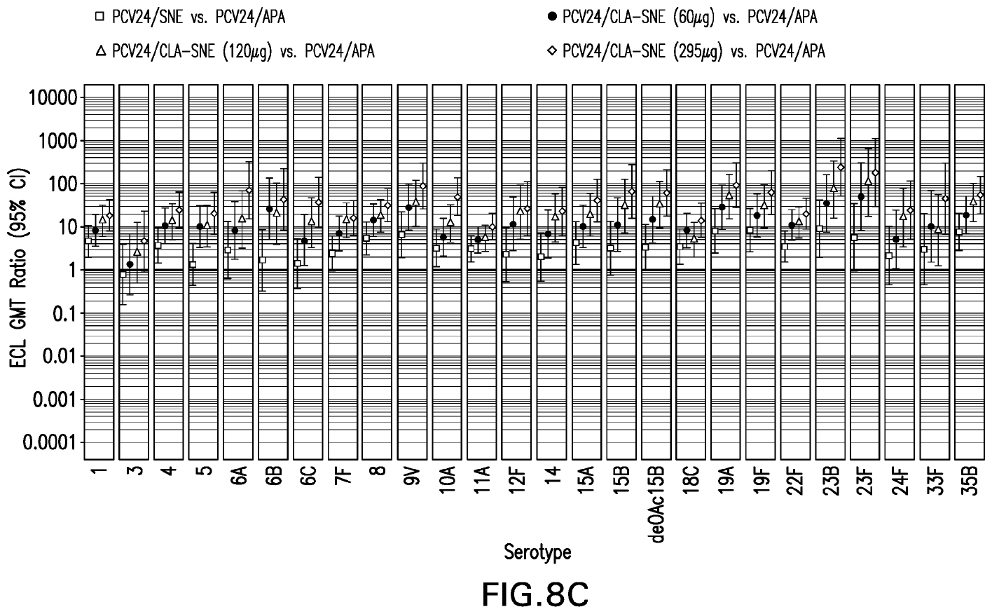

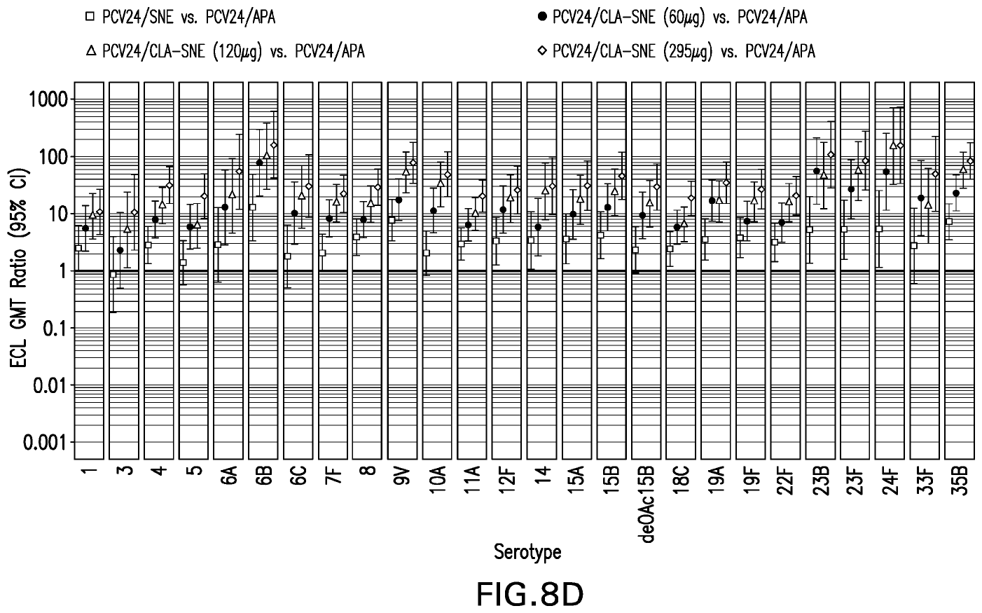

As shown in FIG. 8A, at post dose 2 (day 42), PCV24 formulated with CLA-LNP results in equal or better immunogenicity as compared to PCV24 formulated with APA (circles). PCV24 formulated with CLA-SNE (295 μg CLA with 2.5 mg of squalene; 0.25 mg of PS-20; 0.25 mg of SPAN-85 per 0.1 mL dose, shown as triangles) or CLA-SNE (295 μg CLA with 0.5 mg of squalene; 0.05 mg of PS-20; 0.05 mg of SPAN-85 per 0.1 mL dose, shown as diamonds) results in equal or better immunogenicity as compared to PCV24 formulated with APA. PCV13 and PCV24/APA have comparable immunogenicity for the serotypes (STs) in common, except for ST5 which demonstrates higher immunogenicity for PCV24/APA (squares).

PCV24 formulated with CLA-SNE (60 μg CLA with 2.5 mg of squalene; 0.25 mg of PS-20; 0.25 mg of SPAN-85 per 0.1 mL dose, shown as circles), CLA-SNE (120 μg CLA with 2.5 mg of squalene; 0.25 mg of PS-20; 0.25 mg of SPAN-85 per 0.1 mL dose, shown as triangles), or CLA-SNE (295 μg CLA with 2.5 mL of squalene; 0.25 mg of PS-20; 0.25 mg of SPAN-85 per 0.1 mL dose, shown as diamonds) results in equal or better immunogenicity as compared to PCV24 formulated with APA at PD1 (FIG. 8B), PD2 (FIG. 8C) and PD3 (FIG. 8D). Noteworthy, PCV24 formulated with CLA-SNE at 120 μg CLA using 0.08 mg of squalene; 0.008 mg of PS-20; 0.008 mg of SPAN-85 per 0.1 mL dose, results in comparable immunogenicity compared to PCV24 formulated with CLA-SNE at 120 kg (CLA) and formulated with 0.5 mg of squalene; 0.05 mg of PS-20; 0.05 mg of SPAN-85 per 0.1 mL dose or PCV formulated with CLA-SNE at 295 μg CLA with 2.5 mg of squalene; 0.25 mg of PS-20; 0.24 mg of SPAN-85 per 0.1 mL dose (data not shown).

### Example 9: PCV24 Protection from Challenge in Mice

Young female Swiss Webster mice (6-8 weeks old, n=10/group) were intramuscularly (IM) immunized with 0.1 mL of a 24-valent pneumococcal conjugate vaccine (PCV24) on days 0, 14, and 28. PCV24 was dosed at 0.4 μg PnPs (1, 3, 4, 5, 6A, 6B, 7F, 8, 9V, 10A, 11A, 12F, 14, 15A, de-O-Ac-15B, 18C, 19A, 19F, 22F, 23B, 23F, 24F, 33F and 35B) each conjugated to CRM197 per immunization. PCV24 was formulated with several adjuvant systems, as described in Table 7. Mice were observed daily by trained animal care staff for any signs of illness or distress. The vaccine formulations in mice were deemed to be safe and well tolerated, as no vaccine-related adverse events were noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc. (Kenilworth, NJ, USA).

On day 53, mice were anesthetized with isoflurane and intratracheally challenged with Streptococcus pneumoniae serotype 24F. Briefly, exponential phase cultures of Streptococcus pneumoniae were centrifuged, washed, and suspended in sterile PBS. 4.6×10⁶ cfu of Streptococcus pneumoniae in 0.1 mL of PBS were placed in the throat of mice hung upright by their incisors. Aspiration of the bacteria was induced by gently pulling the tongue outward and covering the nostrils. Mice were weighed daily and euthanized if weight loss exceeded 20% of starting weight. Blood was collected at 24 hours, 48 hours, and 72 hours post challenge to assess for bacteremia. Mice were observed at least twice daily by trained animal care staff for any signs of illness or distress.

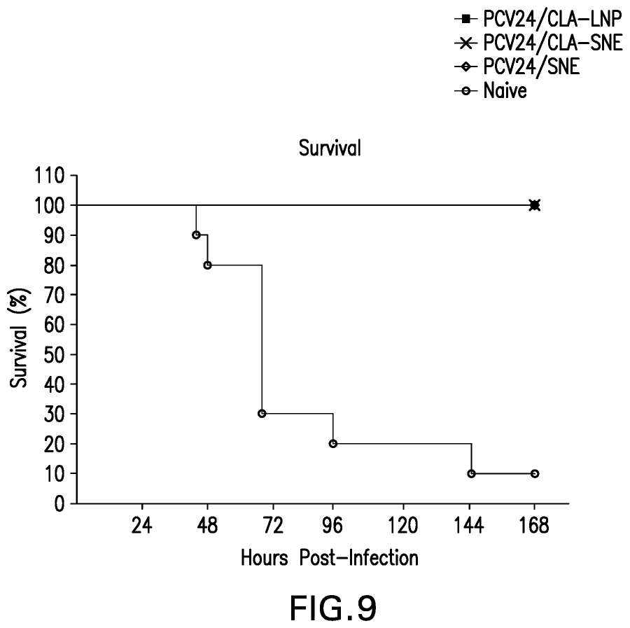

Mice immunized with PCV24 containing adjuvanted (CLA-LNP, CLA-SNE, or SNE alone) vaccines were protected from serotype 24F intratracheal challenge (FIG. 9). All mice immunized with the PCV24 formulation containing adjuvants had 100% survival rate compared to 10% survival rate of naive mice at 7 days post-challenge. This data demonstrates that PCV24 with adjuvant formulations were able to protect mice from serotype 24F IT challenge.

TABLE 7
PCV24 Formulated with Adjuvants Used to Immunize Mice in
Streptococcus pneumoniae 24 F Intratracheal Challenge Model
Formulations
PCV24 in 10 mM L-histidine, 10 mM Tris,
5% (w/v) sucrose, 75 mM NaCl, 0.05% w/v PS-20
[1200 μg/mL CLA-LNP]
PCV24 in 10 mM L-histidine, 75 mM NaCl, 0.05% w/v PS-20
[1200 μg/mL CLA-SNE (25 mg/mL of Squalene; 2.5 mg/mL of PS-20;
2.5 mg/mL of SPAN-85)]
PCV24 in 10 mM L-histidine, 75 mM NaCl, 0.05% w/v PS-20
[SNE (25 mg/mL of squalene; 2.5 mg/mL of PS-20;
2.5 mg/mL of SPAN-85)]

### Example 10: Impact of Time and Temperature on Nanoemulsion Formulation Physical Stability Using NTA and DLS

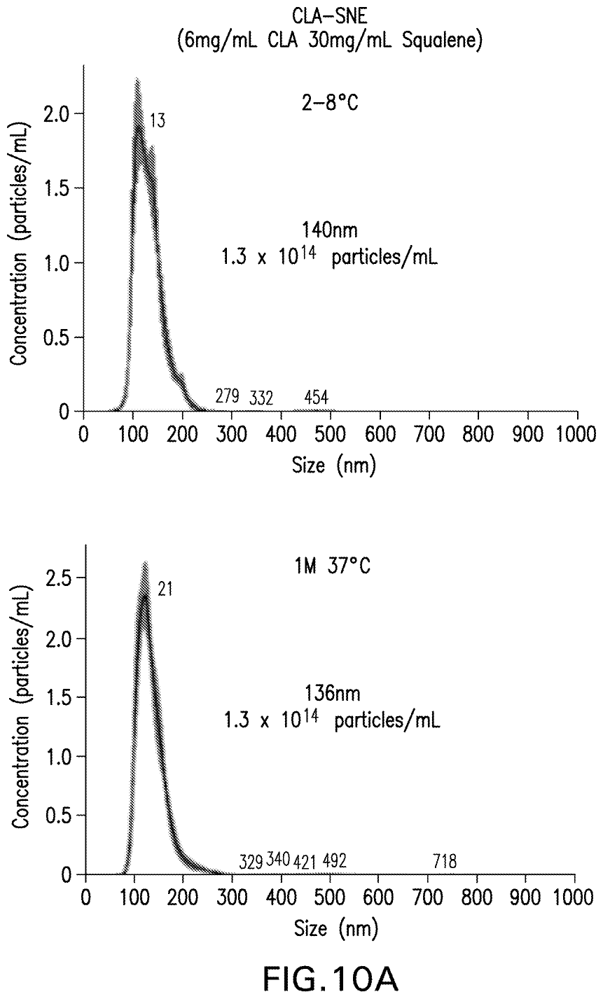

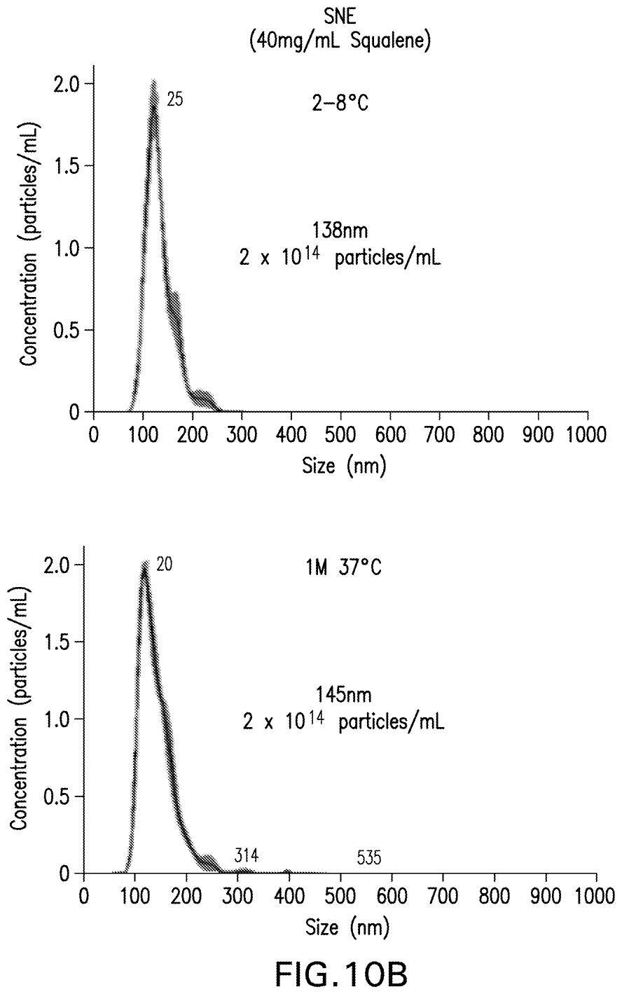

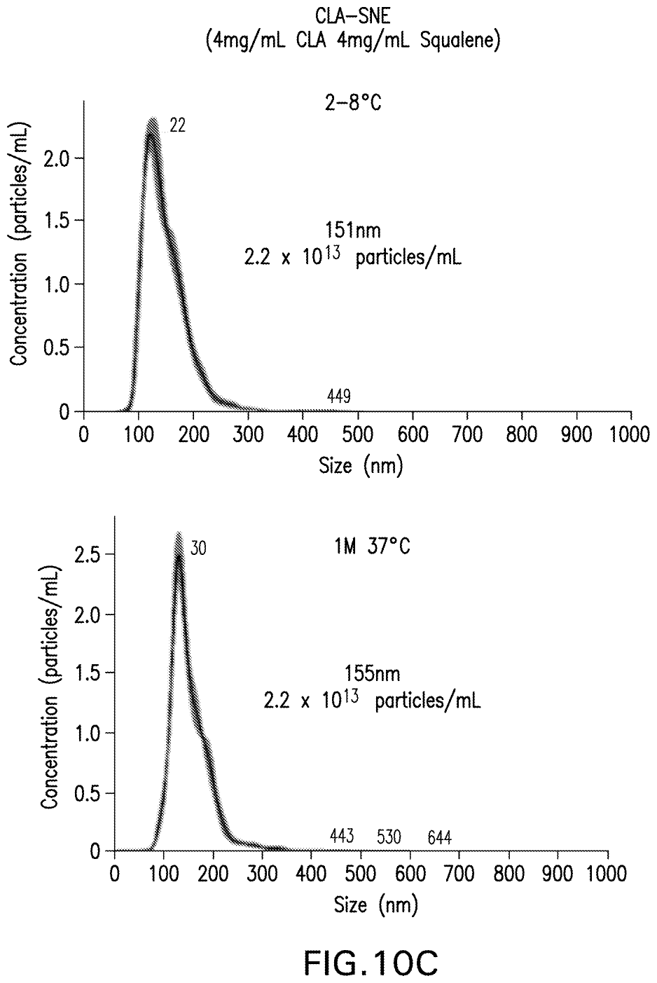

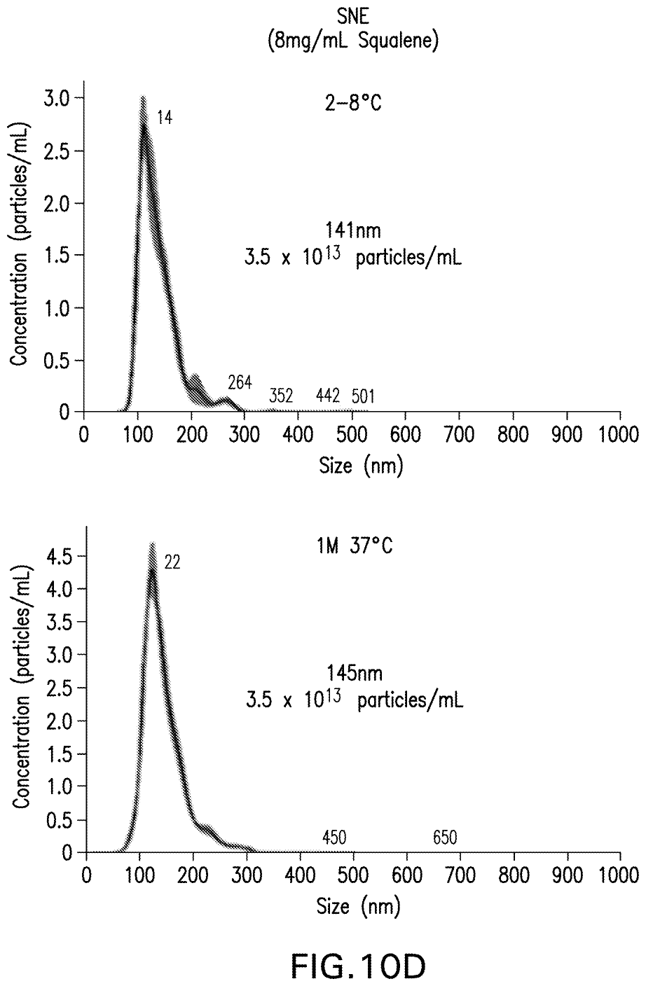

As shown in FIG. 10, to assess stability of the nanoemulsion systems (CLA-SNE or SNE), prepared as described in Examples, supra, nanoparticle tracking analysis (NTA) was utilized. The technique collects videos of directly tracked nanoparticle populations as they move by Brownian motion to extrapolate particle size and concentration. A class 1, 635 nm laser focuses an 80 mm red laser beam through the liquid sample, illuminating particles as rapidly diffusing points of light. A CCD camera records a 30 frame per second video to track the movement of each individual illuminated particle over time. The system software identifies the center of each individual particle from the video and tracks the distance independently traversed to determine the mean square displacement. This tracking was performed simultaneously for every particle within the sample population in each frame until the raw data collected from the entire video was analyzed. By simultaneously measuring the mean square displacement of every individual particle tracked, its diffusion coefficient (Dt) and the spherical equivalent hydrodynamic radius (rh) were determined by applying the Stokes-Einstein equation. The software then represents this accumulated data as a particle size and concentration distribution. Raw data information on not only particle size and concentration, but also intensity, or brightness of the individual particle were gathered. Taken together the data were fitted and plotted individually as particle intensity relative to particle size, and particle concentration relative to particle size, and then on three-dimensional contour plots comparing particle size, concentration, and intensity of all particle populations.

Upon exposure of the nanoemulsion formulations (CLA-SNE or SNE) to 4° C., 25° C. and 37° C. for up to 1 month, no significant change in particle concentration or size distribution of the nanoemulsions was observed as evaluated using NTA (FIG. 10).

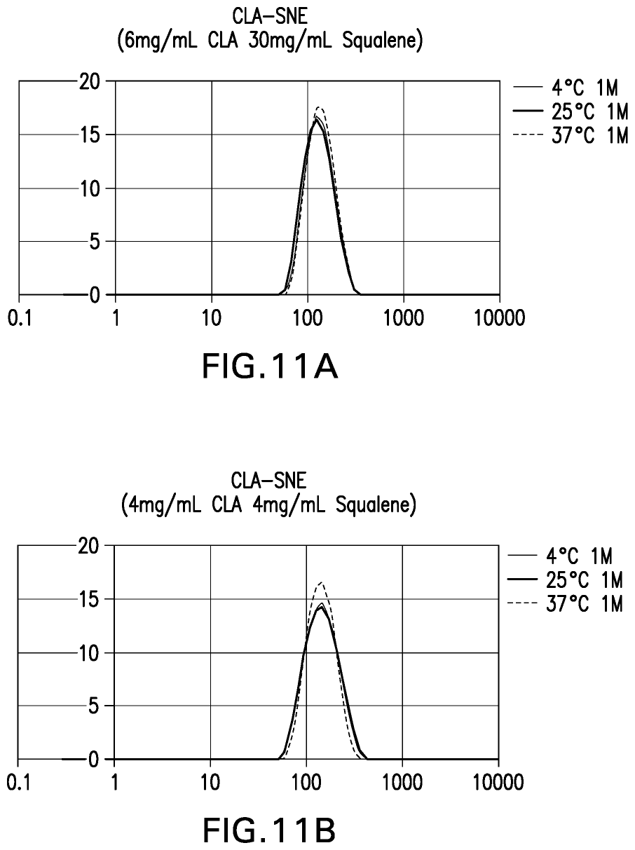

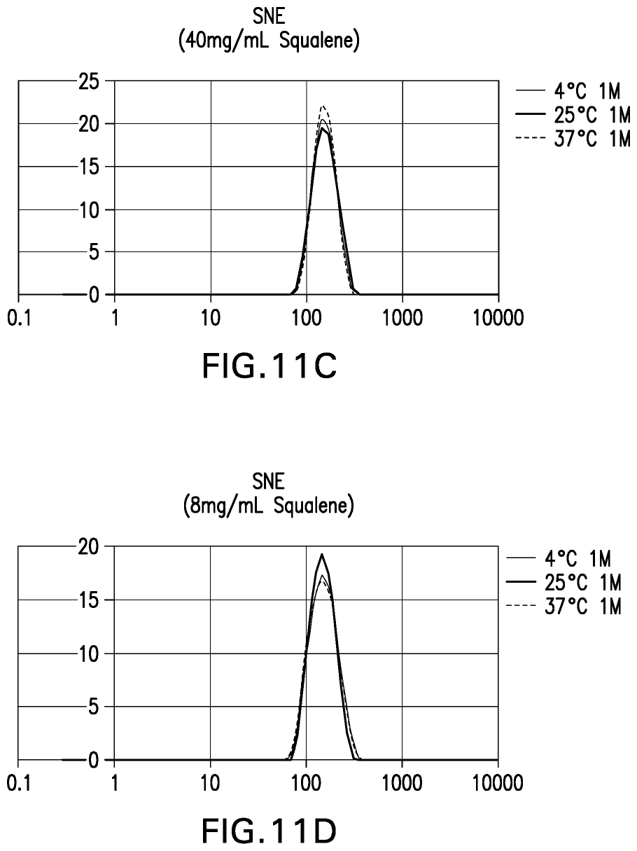

A nanoemulsion may be susceptible to aggregation within the 10-1000 nm particle size range, thereby making DLS a suitable stability indicating technique for assessing and quantitating aggregation phenomena. To assess stability of the nanoemulsion systems, prepared as described in examples, supra, dynamic light scattering (DLS) was utilized to measure the average particle size distribution. DLS instruments use a laser to illuminate particles in a solution and then examine the changes in intensity of the scattered light over time as a result of Brownian motion. The correlation of the scattered light intensity over time to the intensity at time zero results in an exponential decay curve or correlation function. The rate of decay in the correlation function with respect to time is much faster for smaller particles than larger particles and this forms the basis for calculation of the particle sizes. Upon exposure of the nanoemulsion to 4° C., 25° C. or 37° C. for up to 1 month, no change in the size distribution of the nanoemulsions was observed by DLS (FIG. 11). The Z-average remained around 110 nm to 180 nm for the CLA-SNE or SNE.

### Example 11: Impact of Time and Temperature on Nanoemulsion Formulation Chemical Stability Using UPLC-CAD

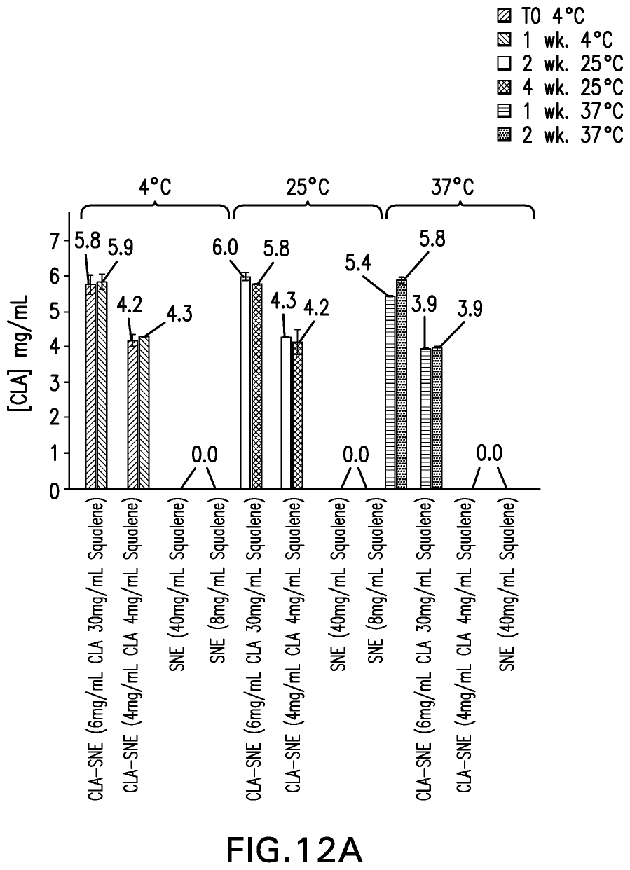

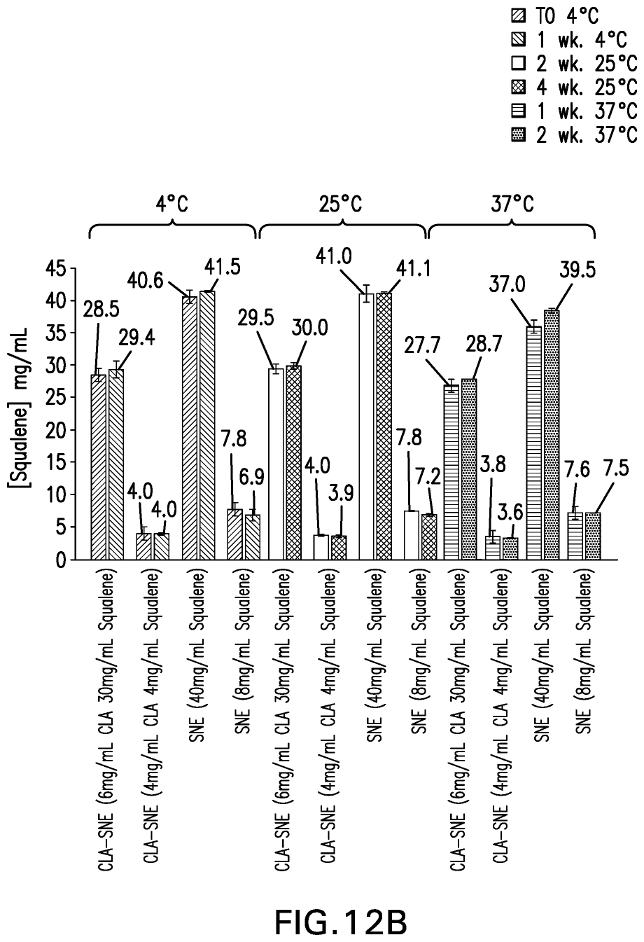

To assess the chemical stability of the nanoemulsion systems, prepared as described in the Examples, supra, ultra-performance liquid chromatography coupled with a charge aerosol detector (UPLC-CAD) was utilized to measure the stability of the CLA (CLA-SNE only) and squalene concentration upon storage at 4° C., 25° C. and 37° C. for 1 month. Upon exposure of the nanoemulsion to 4° C., 25° C. and 37° C. for up to 1 month, the concentration of CLA (FIG. 12A) or squalene (FIG. 12B) in the SNE was not impacted. Moreover, UPLC-CAD can quantitate the production of degradation products because of chemical breakdown of either squalene or CLA. Upon exposure of the SNE or CLA-SNE adjuvant systems to elevated temperature, no detectable degradation peaks were observed indicating that the squalene and CLA components of CLA-SNE and SNE adjuvant systems have excellent thermal stability (data not shown).

### Example 12: Impact of the Stable Emulsion System (+/−CLA) on the Stability of a Pneumococcal Conjugate Vaccine

Individual pneumococcal polysaccharide-carrier protein conjugates prepared utilizing reductive amination solvents (aprotic DMSO) were used for the formulation of PCV24 at 192 μg/mL, as described in the Examples, supra.

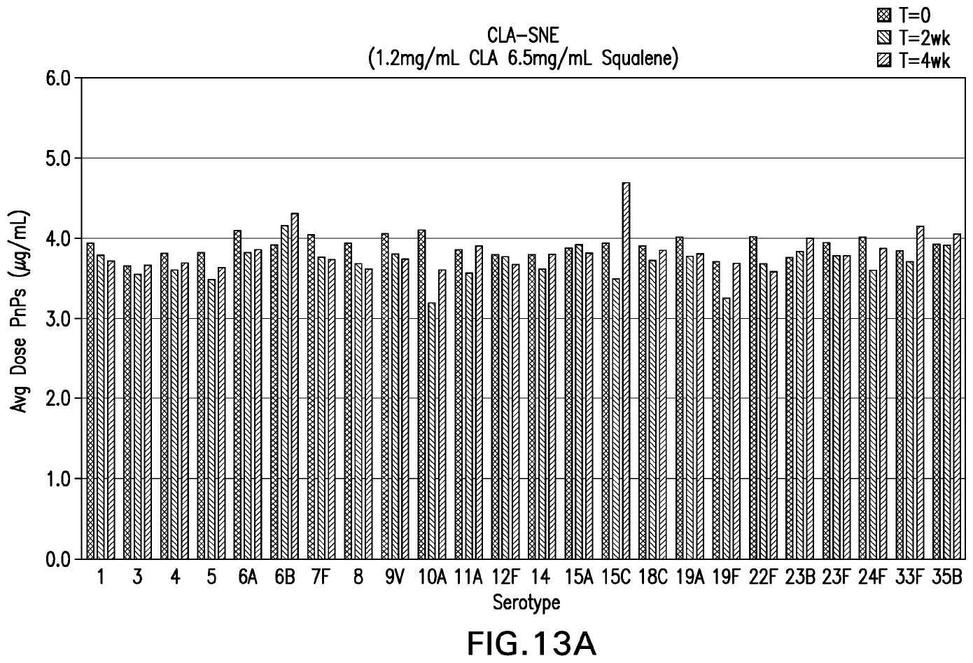

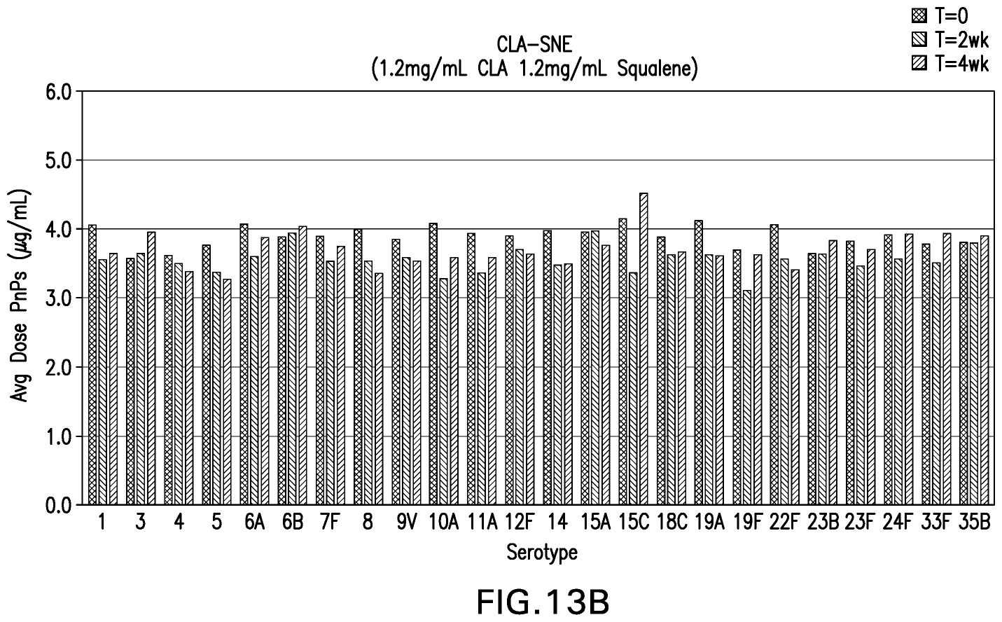

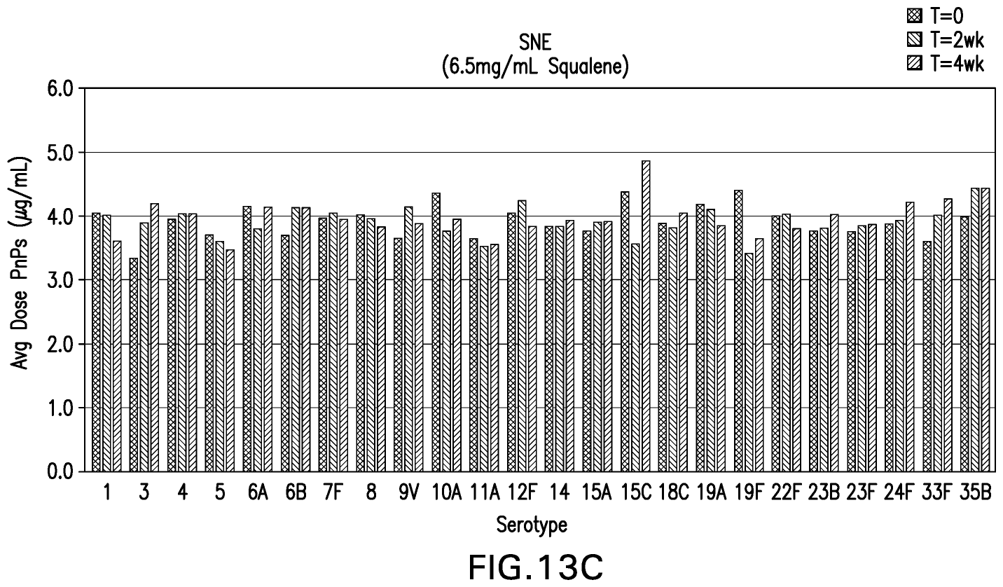

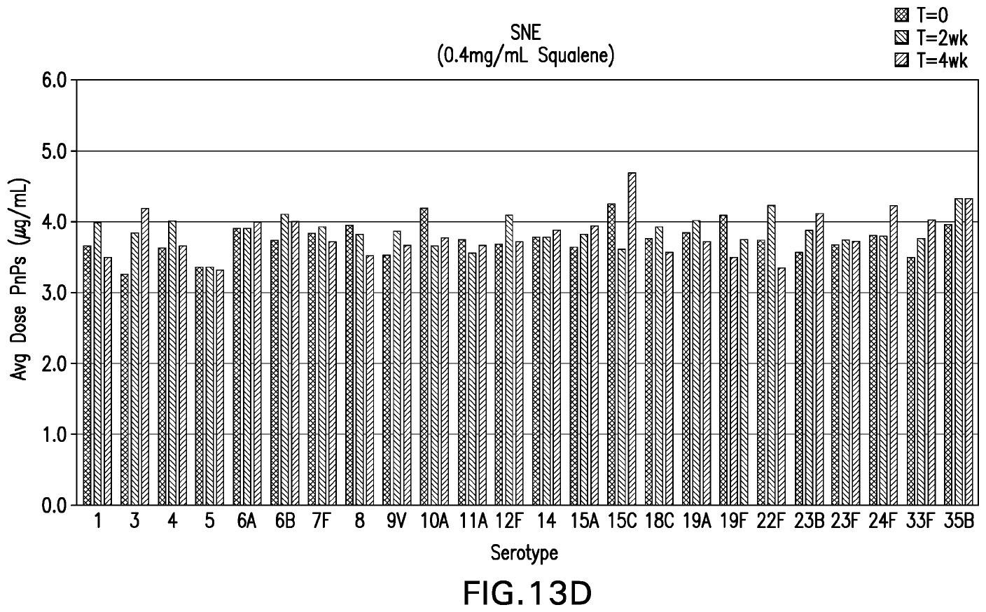

The PCV24 composition was combined with different adjuvant systems, described in Table 8, in a glass container and placed at 4° C. up to 30 days. The formulation demonstrated good stability and coformulation with CLA-SNE (1.2 mg/mL CLA-SNE [6.5 mg/mL of squalene; 0.65 mg/mL of PS-20; 0.65 mg/mL of SPAN-85] or 1.2 mg/mL CLA-SNE [1.2 mg/mL of squalene; 0.12 mg/mL of PS-20; 0.12 mg/mL of SPAN-85] or SNE ([6.5 mg of squalene; 0.65 mg of PS-20; 0.65 mg of SPAN-85] or [0.4 mg of squalene; 0.04 mg of PS-20; 0.04 mg of SPAN-85] did not impact the stability of the pneumococcal polysaccharide-carrier protein conjugate dose (FIG. 13A-13D) using a fluorescence based ELISA assay.

TABLE 8
Summary of PCV24 Formulations Prepared
with CLA-SNE or SNE Adjuvants
Formulations
PCV24 in 10 mM L-histidine, 75 mM NaCl, 0.05% w/v PS-20
[1.2 mg/mL CLA-SNE (6.5 mg/ml of squalene; 0.65 mg/mL
of PS-20; 0.65 mg/mL of SPAN-85)]
PCV24 in 10 mM L-histidine, 75 mM NaCl, 0.05% w/v PS-20
[1.2 mg/mL CLA-SNE (1.2 mg/mL of squalene; 0.12 mg/mL of PS-20;
0.12 mg/mL of SPAN-85)]
PCV24 in 10 mM L-histidine, 75 mM NaCl, 0.05% w/v PS-20
[SNE (6.5 mg of squalene; 0.65 mg of PS-20; 0.65 mg of SPAN-85)]
PCV24 in 10 mM L-histidine, 75 mM NaCl, 0.05% w/v PS-20
[SNE (0.4 mg of squalene; 0.04 mg of PS-20; 0.04 mg of SPAN-85)]

### Example 13: PCV21 Immunogenicity in Adult Rhesus Macaques

PCV21 (Serotypes-3, 6A, 7F, 8, 9N, 10A, 11A, 12F, 15A, deO-Acetylated-15B (deOAc15B), 16F, 17F, 19A, 20, 22F, 23A, 23B, 24F, 31, 33F and 35B each individually conjugated to CRM197) was also assessed in an adult rhesus macaque immunogenicity model, as described in the Examples, supra. Rhesus macaques were intramuscularly immunized with PCV21 alone or PCV21 formulated with CLA formulated as an SNE (CLA-SNE) (Table 9) on days 0, and 28. PCV21 was dosed at 1.0 μg PnPs in a 0.25 mL volume per immunization. Sera were collected prior to study start (pre-immune, day 0) and on days 14 (PD1), 28 (PD1) and 42 (PD2).

TABLE 9
PCV21 Formulations Evaluated in an Adult
Rhesus Macaque Immunogenicity Model
Formulation
PCV21 in 20 mM L-histidine, 150 mM NaCl,
0.1% w/v PS-20 [No Adjuvant]
PCV21 in 20 mM L-histidine, 112.5 mM NaCl,
0.75% w/v PS-20
[1.2 mg/mL CLA-SNE (1.2 mg/mL
of squalene; 0.12 mg/mL
of PS-20; 0.12 mg/mL of SPAN-85)]

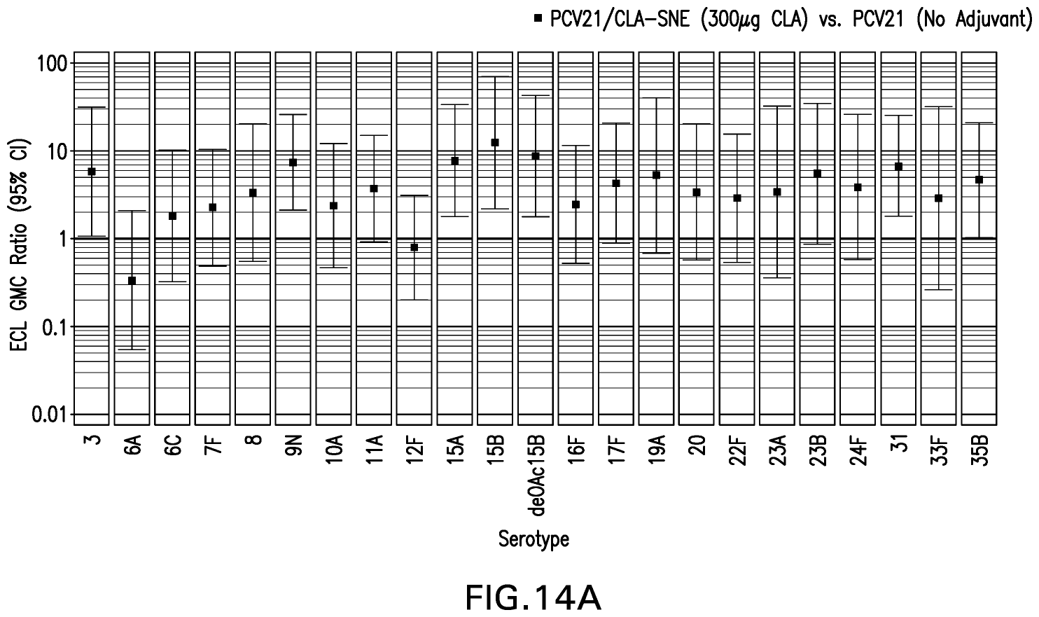

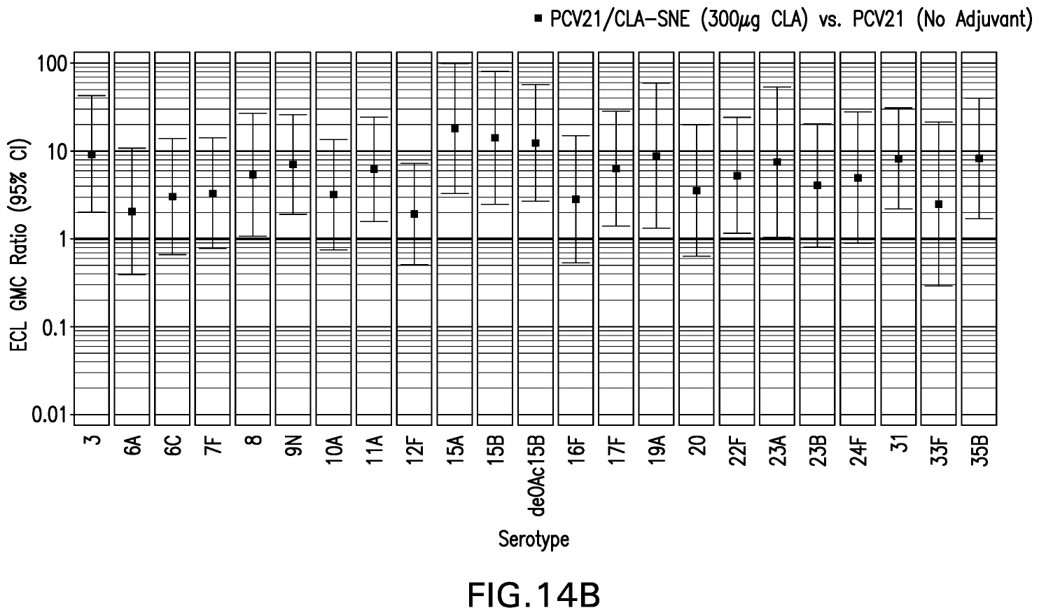

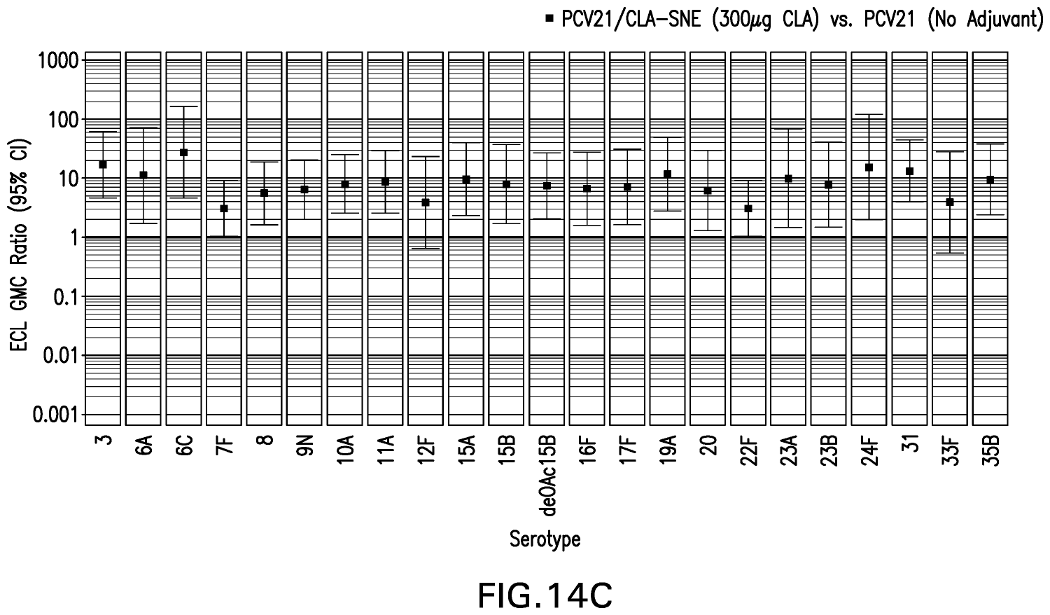

To assess serotype-specific IgG immunogenicity responses in a 21-valent vaccine, a multiplexed electrochemiluminescence (ECL) assay was developed. This assay was developed for use with rhesus serum based on previous assays described by Marchese et al. and Skinner et al. (Marchese R. D. et al., Clin. Vaccine Immunol. (2009) 16(3):387-96 and Skinner, J. M. et al., Vaccine (2011) 29(48):8870-8876). Technology developed by MesoScale Discovery (a division of MesoScale Diagnostics, LLC, Gaithersburg, MD) utilizes pneumococcal serotype polysaccharides (3, 6A, 6C, 7F, 8, 9N, 10A, 11A, 12F, 15A, 15B, deOAc15B, 16F, 17F, 19A, 20, 22F, 23A, 23B, 24F, 31, 33F and 35B) coated on spots in a 96-well plate and a SULFO-TAG™ labeled antibody that emits light upon electrochemical stimulation. SULFO-TAG™_labeled anti-human IgG was used as the secondary antibody for testing rhesus serum samples. The IgG concentration was interpolated off the Reference Serum Standard 007sp for FIGS. 14A, 14B and 14C. Functional antibody was determined through multiplexed opsonophagocytic assays (MOPA) based on previously described protocols available from the UAB Pneumococcal Reference Laboratory (University of Alabama Reference Laboratory at Birmingham Bacterial Respiratory Pathogen Reference Library) and Opsotiter® 3 software owned by and licensed from University of Alabama (UAB) Research Foundation (See, Caro-Aguilar I. et al., Vaccine (2017) 35(6):865-72 and Burton R. L. and Nahm M. H. Clin. Vaccine Immunol. (2006) 13(9):1004-9).

PCV21 formulated with the CLA-SNE prepared at 1.2 mg/mL CLA-SNE with (1.2 mg/mL of squalene; 0.12 mg/mL of PS-20; 0.12 mg/mL of SPAN-85) was found to be immunogenic in adult rhesus macaques and resulted in higher immunogenicity at post dose 1 (Day 14—FIG. 14A; Day 28—FIG. 14B) and post dose 2 (Day 42 FIG. 14C) as compared to PCV21 formulated without an adjuvant. PCV21 formulated with CLA-SNE at a dose level of 300 μg results in equal or better immunogenicity as compared to PCV21 formulated without an adjuvant for post dose 1 (PD1) and post dose 2 (PD2).

These same formulations generated functional antibodies which killed vaccine-type bacterial strains at all time points tested and again the CLA-SNE boosted PCV21 response as compared to PCV21 formulated alone. All study time points were tested in MOPA as pooled or individual samples. Not much difference was observed in the OPA titers between PD1 and PD2 (Data not Shown).

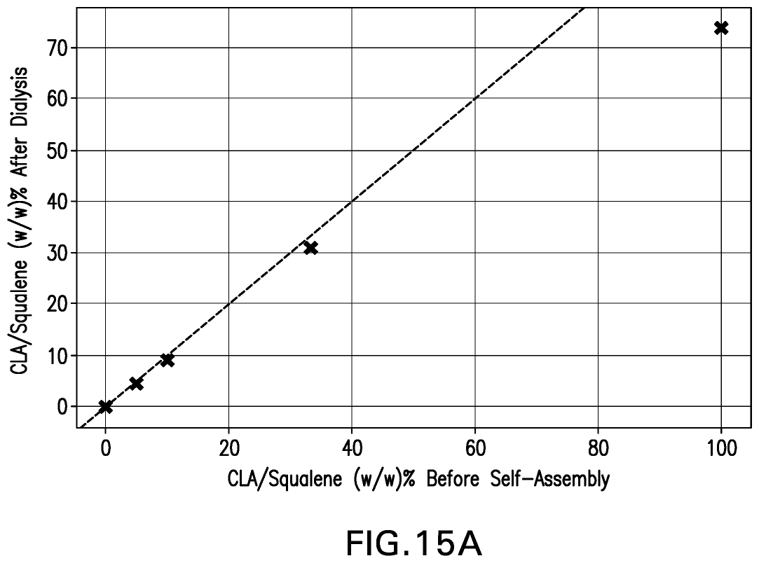

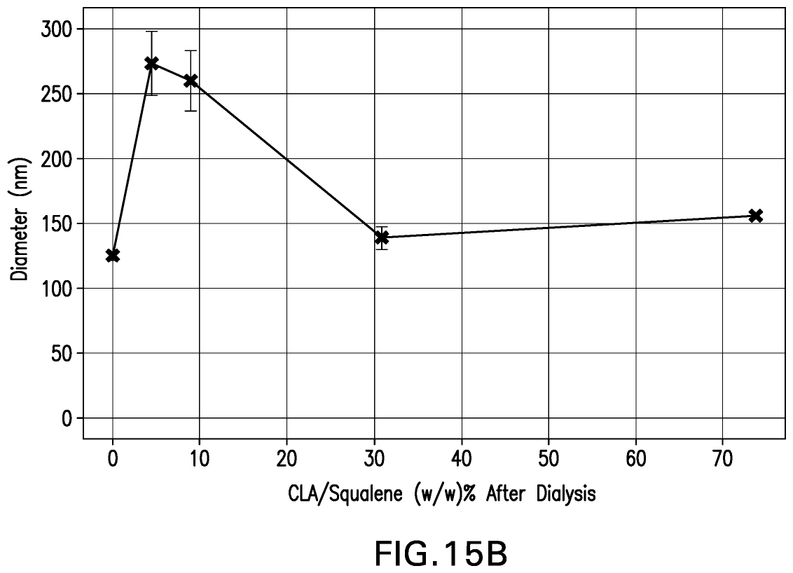

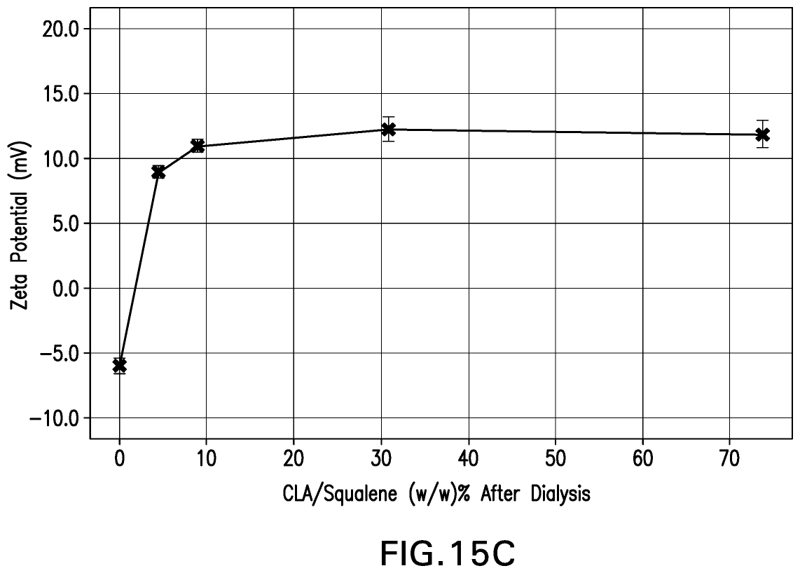

PCV21 immunized adult rhesus macaque sera were evaluated for cross reactivity to other Streptococcus pneumoniae bacteria. PCV21 immunized macaque sera had cross reactivity with serotypes 6C (FIGS. 14A, 14B and 14C) and 15B (FIGS. 14A, 14B and 14C). The cross reactivity to 6C is likely due to immunization with polysaccharide conjugate 6A-CRM197 as part of a multivalent PCV24 (Cooper D, Yu X, Sidhu M, Nahm M H, Fernsten P, Jansen K U). The 13-valent pneumococcal conjugate vaccine (PCV13) elicits cross-functional opsonophagocytic killing responses in humans to Streptococcus pneumoniae serotypes 6C and 7A. Vaccine. 2011; 29:7207-11). Similarly, immunization with polysaccharide conjugates de-O-Ac-15B-CRM197 (deOAc15B-CRM197) as part of a multivalent PCV resulted in cross reactivity to serotype 15C (Rajam et a1., Clinical and Vaccine Immunology, 2007, 14(9):1223-1227).

### Example 14: Preparation of a Stable Nanoemulsion (SNE) Adjuvant System with and without the Cationic Lipid, (13Z,16Z)—N, N-dimethyl-3-nonyldocosa-13, 16-dien-1-amine) by Microfluidic Nanoemulsion Self-Assembly (MNS)

The stable nanoemulsion adjuvant is prepared with and without the ionizable cationic lipid (13Z,16Z)—N, N-dimethyl-3-nonyldocosa-13, 16-dien-1-amine), also referred to as CLA (FIG. 1). The microfluidic nanoemulsion self-assembly (MNS) process used to prepare an SNE. The SNE is multi-component emulsion formulation consisting of 3 stabilizing ingredients; squalene, sorbitan trioleate (SPAN-85), and polysorbate-20 (PS-20) with CLA (referred to as CLA-SNE, Table 10) or without a CLA (referred to as SNE, Table 11). The biophysical characteristics (e.g., particle size, chemical compositions) and stability of MNS-prepared-SNE/CLA-SNE formulations is very similar to that of the High Pressure fine homogenization process for preparation of SNE/CLA-SNE formulations described in Example 3. Essentially, the microfluidic nanoemulsion self-assembly (MNS) process described in this example is an alternative process for preparing a stable nanoemulsion (SNE) adjuvant system. The nanoparticle self-assembly process described in this example was done using a “microfluidics” ethanol/aqueous mixing instrument. However, the ethanol/aqueous stream nanoparticle self-assembly process described in this invention is not limited by “microfluidic” mixing. Mixing larger volume streams of hydrophobic solvents with aqueous solutions can be accomplished using a Tee-mixing process outlined in Example 4.

SNE MNS formulations can generally be prepared by dissolving the cationic lipid, squalene, SPAN-85, and PS-20 at the targeted concentrations into an appropriate non-aqueous solvent such as ethanol. The self-assembly procedure involves combining a stream of the ethanol dissolved hydrophobic emulsion components with a stream of the aqueous emulsion solution. As the two solvent streams combine, the hydrophobic molecules (i.e., the cationic lipid, squalene, SPAN-85, and PS-20) interact with the aqueous solvent. The molecules then assemble themselves into an emulsion of nanosized particles, as described below. Following the formation of the self-assembled emulsion of nanoparticles, the residual ethanol can be removed from the stable squalene emulsion by several suitable means. In this example, the ethanol was reduced to less than 0.1% (w/v) by overnight dialysis with the aqueous buffer. The resulting SNE formulation was sterilized by filtration through a 0.2 μm pore size sterilization filter. Several process parameters within each step, such as order or addition, mixing times, temperature, concentration of non-aqueous components, concentrations of aqueous buffer components, aqueous pH, non-aqueous to aqueous solution mixing ratio, total flow rate, and waste discard volumes were controlled to yield SNE adjuvant systems with the desired attributes.

TABLE 10
Composition of CLA-SNE Adjuvant Prepared by MNS

| | | Content of | Molecular | Content |
|---|---|---|---|---|
| | | Each Lipid | Weight | of Each |
| Component | Description | (Mole %) | (g/mol) | Lipid (Mass %) |
| CLA | (13Z,16Z)-N,N-dimethyl-3- | 4-45% | 475.9 | 4-46% |
| | nonyldocosa-13,16-dien-1- | | | |
| | amine | | | |
| Squalene | squalene | 52-89% | 410.72 | 45-80% |
| SPAN-85 | sorbitan trioleate | 2-4% | 957.5 | 4-8% |
| PS-20 | polysorbate-20 | 2-3% | 1228 | 4-8% |

| | | |
|---|---|---|
| Buffer Matrix | 20 mM Histidine, pH 5.8 | N/A |

TABLE 11
Composition of SNE Adjuvant Prepared by MNS

| | | | Molecular | Content of |
|---|---|---|---|---|
| | | Content of Each | Weight | Each Lipid |
| Component | Description | Lipid (Mole %) | (g/mol) | (Mass %) |
| Squalene | squalene | 92.9% | 410.72 | 83.3% |
| SPAN-85 | sorbitan trioleate | 4.0% | 957.5 | 8.3% |
| PS-20 | polysorbate-20 | 3.1% | 1228 | 8.3% |

| | | |
|---|---|---|
| Buffer Matrix | 20 mM Histidine, pH 5.8 | N/A |

### Formulation Preparation Using Microfluidic Nanoemulsion Self-Assembly

In this example, one SNE and four CLA-SNE formulations were prepared by the microfluidic nanoemulsion self-assembly procedure in 20 mM histidine pH 5.8 for biophysical characterization. The self-assembled nanoemulsion process starts with 15 mg/mL squalene, 1.5 mg/mL SPAN-85 and 1.5 mg/mL PS-20 completely dissolved in ethanol. In addition, each of the ethanol solutions described above also contained CLA at either 0.75, 1.5, 5.0 or 15.0 mg CLA/mL. Thus, the initial “target” CLA/squalene (w/w) % for all five formulations would be 0.0, 5.0, 10.0, 33.3. and 100 (w/w) % CLA/squalene. The aqueous buffer for all the formulations was 20 mM histidine at pH 5.8. A benchtop NanoAssemblr™ instrument from Precision NanoSystems, Inc. (Vancouver, BC, Canada) was used to self-assemble the one SNE and four CLA-SNE adjuvant nanoemulsions

The self-assembled squalene nanoparticle formulations were prepared in the following manner. A 1 mL syringe was filled with a little over 0.7 mL of the hydrophobic compound mixture dissolved in ethanol, while a 3 mL syringe was filled with a little over 1.4 mL of the aqueous 20 mM histidine pH 5.8 buffer. After both syringes were loaded with the appropriate amount of solution, the syringes were attached to the NanoAssemblr™ instrument. The following microfluidic mixing parameters were programed into the NanoAssemblr™: a) Total volume=2 mL, b) Flow rate ratio=2:1 (aqueous to ethanol), c) Total Flow Rate=12 mL/min, d) Start waste volume=0.25 mL, and e) End waste volume=0.05 mL. The instrument was activated to start the ethanol and aqueous solution mixing process in as little as a few seconds. Approximately 2.0 mL of post-mixing nanoparticle emulsion in approximately 30% ethanol was collected in a 15 mL Falcon tube for each of the 5 formulations. A fresh NanoAssemblr™ mixing cartridge was used for each of the 5 different SNE and CLA-SNE formulations described above. The ethanol concentration is reduced in each formulation by overnight dialysis. After dialysis, all of the samples were stored at 4° C. prior to analytical characterization.

### Analytical Characterization

The cationic lipid, CLA, and squalene were equally incorporated into the squalene CLA-SNE nanoparticles prepared by MNS as shown in FIG. 15A. The CLA/squalene (w/w) % ratio after dialysis (i.e., the y axis) to remove the process ethanol was compared to the CLA/squalene (w/w) % before self-assembly (i.e., the x axis) while in the ethanol solution for all the formulation samples described in this example. The “measured” CLA/squalene (w/w) % after MNS and dialysis was equal to the “target” (w/w) % up to at least 35 (w/w) %. Even at a 100% “target” CLA/squalene (w/w) % prior to self-assembly over 70% of the available CLA is incorporated into the CLA-SNE nanoparticles relative to the squalene content in the MNS prepared nanoparticle emulsion. The CLA/squalene (w/w) % ratios were measured by reverse phase UPLC-CAD. CLA is clearly incorporated into CLA-SNE by prepared by microfluidic nanoemulsion self-assembly (MNS) process.

The intensity weighted Z-average DLS diameters of the CLA-SNE formulations prepared the MNS process were measured using a Malvern ZetaSizer Ultra. Aliquots of post-dialyzed CLA-SNE samples from each formulation were diluted at either 50- or 100-fold in 2.0 mL 20 mM histidine pH 5.8 buffer. Average DLS diameter and standard deviation was plotted versus the measured post-dialysis CLA/squalene (w/w) % for each formulation and is shown in FIG. 15B. Three DLS measurements were made at room temperature for each formulation. The standard deviation bars are show unless the standard deviation is less that data point image. The intensity weight Z-average DLS diameters of MNS prepared CLA-SNE ranged from approximately 150 to 280 nm which is similar to CLA-SNE nanoparticles prepared by high-pressure homogenization. Varying MNS process parameters such as those described above in this example were controlled to yield CLA-SNE adjuvant systems with the desired diameters.

The measured Zeta Potential of CLA-SNE squalene nanoparticle formulations at pH 5.5 prepared the MNS process are shown in FIG. 15C. The Zeta Potential was measured using a Malvern ZetaSizer Ultra. Aliquots of post-dialyzed CLA-SNE samples from each formulation were diluted at either 50 or 100× in 2.0 mL of 20 mM citrate BIS TRIS propane buffer at pH 5.5. Three Zeta potential measurements were made at room temperature for each formulation. The standard deviation bars are show unless the standard deviation is less that data point image. The Zeta Potential of the 0 (w/w) % CLA CLA-SNE formulation, i.e. no CLA, was around −5 mV. As illustrated in FIG. 15C, addition of CLA significantly increased the nanoparticle Zeta Potential to around +10 mV.

### Example 15: Optimization of CLA-SNE Preparation by Alteration of the Aqueous Phase pH

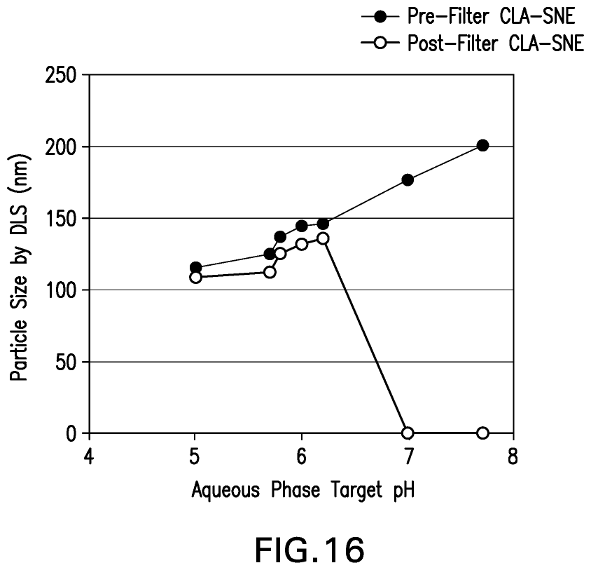

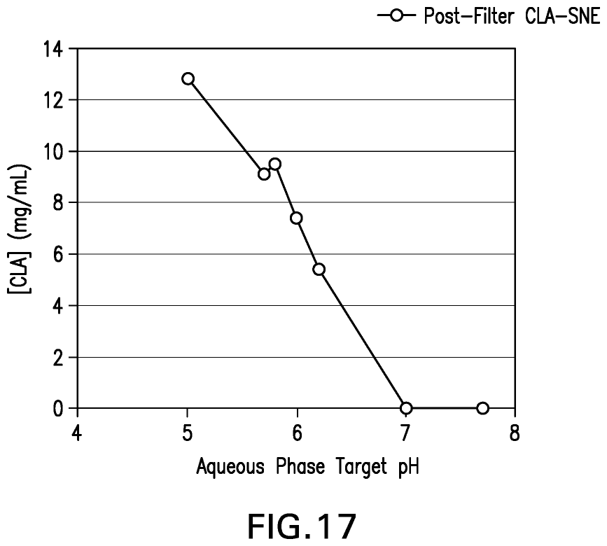

The CLA-SNE process involves the use of a reversible cationic CLA molecule with an observed pKa of 6.4. Addition of CLA to the SNE preparation process and final matrix with a pH of 5.8 results in the protonation of CLA, which functions to give an overall net positive charge to CLA-SNE particles as well as any intermediates of the preparation process. CLA-SNE preparation culminates in a 0.8/0.2 μm filtration event, a process step which had proved difficult to perform, with significant filter fouling and low product yield consistently observed. However, the filtration of SNE did not demonstrate the same magnitude of these filtration challenges and was a more efficient nanoemulsion filtration step. Importantly, without CLA present SNE does not carry the strong positive charge observed with CLA-SNE. In an effort to prepare an uncharged CLA-SNE for a higher efficiency CLA-SNE filtration step, a series of experiments were performed in which the pH of the aqueous phase (20 mM L-Histidine) was adjusted prior to use in the preparation of CLA-SNE. 20 mM L-Histidine was prepared with pH targets of 5.0, 5.7, 5.8, 6.0, 6.2, 7.0, and 7.7. Each buffer was then used as the aqueous phase during the CLA-SNE preparation process with a target formulation target of 15 mg/mL CLA, CLA-SNE preparation proceeded exactly as described in Example 3. Upon completion of homogenization process step, the particle size of the CLA-SNE intermediate was measured by DLS using a Malvern Panalytical Nano ZS. Filtration with 0.8/0.2 μm PES filter was then performed. Particle size distribution was measured post-filtration by DLS and [CLA] was quantified by UPLC-CAD. For CLA-SNE samples prepared with 20 mM L-histidine with pH 7.0 or 7.7, complete filter fouling was observed immediately upon application of material to the filter and no collection of filtered material was possible making quantification by DLS or UPLC-CAD impossible, values of 0 are reported for illustration purposes. In pre-filtered samples, a trend was observed of increasing particle size by DLS as the pH of the aqueous phase buffer was increased (FIG. 16). This relationship was maintained in post-filter samples with each CLA-SNE sample demonstrating a modest reduction in particle size post-filtration, with the obvious exception of pH 7.0 and 7.7 samples which again demonstrated complete filter fouling immediately and no material recovery was possible. For post-filtered samples, it was observed that [CLA] decreases in the final CLA-SNE material as the pH of the aqueous phase is increased (FIG. 17). This relationship demonstrates that CLA-SNE preparation using an aqueous phase at a lower pH results in an increase in process yield in the terminal filtration and a more efficient SLA-CAN manufacturing process.

1-22. (canceled)

23. A composition comprising:
a) at least one Streptococcus pneumoniae polysaccharide;
b) sorbitan trioleate (SPAN-85);
c) polysorbate-20 (PS-20) or polysorbate-80 (PS-80); and
d) squalene.

24. The composition of claim 23, wherein the at least one Streptococcus pneumoniae polysaccharide is conjugated to a carrier protein.

25. The composition of claim 24, wherein the carrier protein is CRM197.

26. The composition of claim 23, wherein the at least one Streptococcus pneumoniae polysaccharide is selected from the group consisting of serotypes:
4, 6B, 9V, 14, 18C, 19F and 23F;
1, 3, 4, 5, 6A, 6B, 7F, 9V, 14, 18C, 19A, 19F and 23F;
1, 3, 4, 5, 6A, 6B, 7F, 9V, 14, 18C, 19A, 19F, 22F, 23F and 33F;
1, 3, 4, 5, 6A, 6B, 7F, 8, 9V, 10A, 11A, 12F, 14, 15B, 18C, 19A, 19F, 22F, 23F and 33F;
1, 3, 4, 5, 6A, 6B, 7F, 8, 9V, 10A, 11A, 12F, 14, 15A, 15B, 18C, 19A, 19F, 22F, 23B, 23F, 24F, 33F and 35B;
1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B;
1, 3, 4, 5, 6A, 6B, 7F, 8, 9V, 10A, 11A, 12F, 14, 15A, 15C, 18C, 19A, 19F, 22F, 23B, 23F, 24F, 33F and 35B;
3, 6A, 7F, 8, 9N, 10A, 11A, 12F, 15A, 15B, 16F, 17F, 19A, 20A, 22F, 23A, 23B, 24F, 31, 33F and 35B;
3, 6A, 7F, 8, 9N, 10A, 11A, 12F, 15A, de-O-acetylated-15B, 16F, 17F, 19A, 20A, 22F, 23A, 23B, 24F, 31, 33F and 35B;
3, 6A, 7F, 8, 9N, 10A, 11A, 12F, 15A, 15C, 16F, 17F, 19A, 20A, 22F, 23A, 23B, 24F, 31, 33F and 35B;
3, 6A, 7F, 8, 9N, 10A, 11A, 12F, 15A, 15B, 16F, 17F, 19A, 20, 22F, 23A, 23B, 24F, 31, 33F and 35B;
3, 6A, 7F, 8, 9N, 10A, 11A, 12F, 15A, de-O-acetylated-15B, 16F, 17F, 19A, 20, 22F, 23A, 23B, 24F, 31, 33F and 35B;
3, 6A, 7F, 8, 9N, 10A, 11A, 12F, 15A, 15C, 16F, 17F, 19A, 20, 22F, 23A, 23B, 24F, 31, 33F and 35B;
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B.

27. The composition of claim 23, wherein the SPAN-85 concentration is 6 μg/mL-24 mg/mL, the PS-20 or PS-80 concentration is 6 μg/mL-24 mg/mL, and the squalene concentration is 60 μg/mL-240 mg/mL.

28. The composition of claim 23, wherein the SPAN-85 concentration is 6 μg/mL-2.4 mg/mL, the PS-20 or PS-80 concentration is 6 μg/mL-2.4 mg/mL, and the squalene concentration is 60 μg/mL-24 mg/mL.

29. A composition comprising:
a) at least one Streptococcus pneumoniae polysaccharide-carrier protein conjugate comprising a Streptococcus pneumoniae polysaccharide conjugated to a carrier protein;
b) sorbitan trioleate (SPAN-85);
c) polysorbate-20 (PS-20); and
d) squalene;
wherein the at least one Streptococcus pneumoniae polysaccharide is selected from the group consisting of serotypes:
4, 6B, 9V, 14, 18C, 19F and 23F;
1, 3, 4, 5, 6A, 6B, 7F, 9V, 14, 18C, 19A, 19F and 23F;
1, 3, 4, 5, 6A, 6B, 7F, 9V, 14, 18C, 19A, 19F, 22F, 23F and 33F;
1, 3, 4, 5, 6A, 6B, 7F, 8, 9V, 10A, 11A, 12F, 14, 15B, 18C, 19A, 19F, 22F, 23F and 33F;
1, 3, 4, 5, 6A, 6B, 7F, 8, 9V, 10A, 11A, 12F, 14, 15A, 15B, 18C, 19A, 19F, 22F, 23B, 23F, 24F, 33F and 35B;
1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B;
1, 3, 4, 5, 6A, 6B, 7F, 8, 9V, 10A, 11A, 12F, 14, 15A, 15C, 18C, 19A, 19F, 22F, 23B, 23F, 24F, 33F and 35B;
3, 6A, 7F, 8, 9N, 10A, 11A, 12F, 15A, 15B, 16F, 17F, 19A, 20A, 22F, 23A, 23B, 24F, 31, 33F and 35B;
3, 6A, 7F, 8, 9N, 10A, 11A, 12F, 15A, de-O-acetylated-15B, 16F, 17F, 19A, 20A, 22F, 23A, 23B, 24F, 31, 33F and 35B;
3, 6A, 7F, 8, 9N, 10A, 11A, 12F, 15A, 15C, 16F, 17F, 19A, 20A, 22F, 23A, 23B, 24F, 31, 33F and 35B;
3, 6A, 7F, 8, 9N, 10A, 11A, 12F, 15A, 15B, 16F, 17F, 19A, 20, 22F, 23A, 23B, 24F, 31, 33F and 35B;
3, 6A, 7F, 8, 9N, 10A, 11A, 12F, 15A, de-O-acetylated-15B, 16F, 17F, 19A, 20, 22F, 23A, 23B, 24F, 31, 33F and 35B;
3, 6A, 7F, 8, 9N, 10A, 11A, 12F, 15A, 15C, 16F, 17F, 19A, 20, 22F, 23A, 23B, 24F, 31, 33F and 35B;
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B;
and the carrier protein is CRM197.

30. The composition of claim 29, wherein the SPAN-85 concentration is 6 μg/mL-24 mg/mL, the PS-20 concentration is 6 μg/mL-24 mg/mL, and the squalene concentration is 60 μg/mL-240 mg/mL.

31. The composition of claim 29, wherein the SPAN-85 concentration is 6 μg/mL-2.4 mg/mL, the PS-20 concentration is 6 μg/mL-2.4 mg/mL, and the squalene concentration is 60 μg/mL-24 mg/mL.

32. A composition comprising:
a) at least one Streptococcus pneumoniae polysaccharide-carrier protein conjugate comprising a Streptococcus pneumoniae polysaccharide conjugated to a carrier protein;
b) sorbitan trioleate (SPAN-85);
c) polysorbate-20 (PS-20); and
d) squalene;
wherein the at least one Streptococcus pneumoniae polysaccharide is selected from the group consisting of serotypes:
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B;
and the carrier protein is CRM197.

33. The composition of claim 32, wherein the SPAN-85 concentration is 6 μg/mL-24 mg/mL, the PS-20 concentration is 6 μg/mL-24 mg/mL, and the squalene concentration is 60 μg/mL-240 mg/mL.

34. The composition of claim 33 wherein the composition further comprises 5 mM-40 mM histidine at pH 5.1-7.0 and 25 mM-300 mM NaCl.

35. The composition of claim 33 wherein the composition further comprises about 20 mM histidine at about pH 5.8 and about 75 mM NaCl.

36. The composition of claim 32, wherein the SPAN-85 concentration is 6 μg/mL-2.4 mg/mL, the PS-20 concentration is 6 μg/mL-2.4 mg/mL, and the squalene concentration is 60 μg/mL-24 mg/mL.

37. The composition of claim 36 wherein the composition further comprises 5 mM-40 mM histidine at pH 5.1-7.0 and 25 mM-300 mM NaCl.

38. The composition of claim 36 wherein the composition further comprises about 20 mM histidine at about pH 5.8 and about 75 mM NaCl.

39. A composition comprising:
a) at least one Streptococcus pneumoniae polysaccharide;
b) sorbitan trioleate (SPAN-85);
c) polysorbate-20 (PS-20) or polysorbate-80 (PS-80);
d) squalene; and
e) a cationic lipid.

40. The composition of claim 39, wherein the at least one Streptococcus pneumoniae polysaccharide is conjugated to a carrier protein.

41. The composition of claim 40, wherein the carrier protein is CRM197.

42. The composition of claim 39, wherein the cationic lipid is selected from Formula 1: 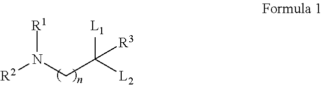
wherein:
R¹ and R² are each methyl;
R³ is H;
n is 1 or 2;
L₁ is selected from C₈-C₂₄ alkyl and C₈-C₂₄ alkenyl; and
L₂ is selected from C₄-C₉ alkyl and C₄-C₉ alkenyl;
or any pharmaceutically acceptable salt or stereoisomer thereof.

43. The composition of claim 39, wherein the cationic lipid is selected from:
(13Z,16Z)—N,N-dimethyl-3-nonyldocosa-13,16-dien-1-amine;
(6Z,9Z,26Z,29Z)—N,N-dimethylpentatriaconta-6,9,26,29-tetraen-18-amine; and
N,N-dimethyl-1-((1S,2R)-2-octylcyclopropyl)heptadecan-8-amine.

44. The composition of claim 39 wherein the cationic lipid is (13Z,16Z)—N,N-dimethyl-3-nonyldocosa-13,16-dien-1-amine.

45. The composition of claim 39, wherein the at least one Streptococcus pneumoniae polysaccharide is selected from the group consisting of serotypes:
4, 6B, 9V, 14, 18C, 19F and 23F;
1, 3, 4, 5, 6A, 6B, 7F, 9V, 14, 18C, 19A, 19F and 23F;
1, 3, 4, 5, 6A, 6B, 7F, 9V, 14, 18C, 19A, 19F, 22F, 23F and 33F;
1, 3, 4, 5, 6A, 6B, 7F, 8, 9V, 10A, 11A, 12F, 14, 15B, 18C, 19A, 19F, 22F, 23F and 33F;
1, 3, 4, 5, 6A, 6B, 7F, 8, 9V, 10A, 11A, 12F, 14, 15A, 15B, 18C, 19A, 19F, 22F, 23B, 23F, 24F, 33F and 35B;
1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B;
1, 3, 4, 5, 6A, 6B, 7F, 8, 9V, 10A, 11A, 12F, 14, 15A, 15C, 18C, 19A, 19F, 22F, 23B, 23F, 24F, 33F and 35B;
3, 6A, 7F, 8, 9N, 10A, 11A, 12F, 15A, 15B, 16F, 17F, 19A, 20A, 22F, 23A, 23B, 24F, 31, 33F and 35B;
3, 6A, 7F, 8, 9N, 10A, 11A, 12F, 15A, de-O-acetylated-15B, 16F, 17F, 19A, 20A, 22F, 23A, 23B, 24F, 31, 33F and 35B;
3, 6A, 7F, 8, 9N, 10A, 11A, 12F, 15A, 15C, 16F, 17F, 19A, 20A, 22F, 23A, 23B, 24F, 31, 33F and 35B;
3, 6A, 7F, 8, 9N, 10A, 11A, 12F, 15A, 15B, 16F, 17F, 19A, 20, 22F, 23A, 23B, 24F, 31, 33F and 35B;
3, 6A, 7F, 8, 9N, 10A, 11A, 12F, 15A, de-O-acetylated-15B, 16F, 17F, 19A, 20, 22F, 23A, 23B, 24F, 31, 33F and 35B;
3, 6A, 7F, 8, 9N, 10A, 11A, 12F, 15A, 15C, 16F, 17F, 19A, 20, 22F, 23A, 23B, 24F, 31, 33F and 35B;
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B.

46. The composition of claim 39, wherein the SPAN-85 concentration is 6 μg/mL-14 mg/mL, the PS-20 or PS-80 concentration is 6 μg/mL-14 mg/mL, the squalene concentration is 60 μg/mL-34 mg/mL and the cationic lipid concentration is 30 μg/mL-2.4 mg/mL.

47. The composition of claim 39, wherein the SPAN-85 concentration is 6 μg/mL-14 mg/mL, the PS-20 or PS-80 concentration is 6 μg/mL-14 mg/mL, the squalene concentration is 60 μg/mL-34 mg/mL and the cationic lipid concentration is 60 μg/mL-2.4 mg/mL.

48. A composition comprising:
a) at least one Streptococcus pneumoniae polysaccharide-carrier protein conjugate comprising a Streptococcus pneumoniae polysaccharide conjugated to a carrier protein;
b) sorbitan trioleate (SPAN-85);
c) polysorbate-20 (PS-20);
d) squalene; and
e) a cationic lipid;
wherein the at least one Streptococcus pneumoniae polysaccharide is selected from the group consisting of serotypes:
4, 6B, 9V, 14, 18C, 19F and 23F;
1, 3, 4, 5, 6A, 6B, 7F, 9V, 14, 18C, 19A, 19F and 23F;
1, 3, 4, 5, 6A, 6B, 7F, 9V, 14, 18C, 19A, 19F, 22F, 23F and 33F;
1, 3, 4, 5, 6A, 6B, 7F, 8, 9V, 10A, 11A, 12F, 14, 15B, 18C, 19A, 19F, 22F, 23F and 33F;
1, 3, 4, 5, 6A, 6B, 7F, 8, 9V, 10A, 11A, 12F, 14, 15A, 15B, 18C, 19A, 19F, 22F, 23B, 23F, 24F, 33F and 35B;
1, 3, 4, 5, 6A, 6B, 7F, 8, 9V, 10A, 11A, 12F, 14, 15A, de-O-acetylated-15B, 18C, 19A, 19F, 22F, 23B, 23F, 24F, 33F and 35B;
1, 3, 4, 5, 6A, 6B, 7F, 8, 9V, 10A, 11A, 12F, 14, 15A, 15C, 18C, 19A, 19F, 22F, 23B, 23F, 24F, 33F and 35B;
3, 6A, 7F, 8, 9N, 10A, 11A, 12F, 15A, 15B, 16F, 17F, 19A, 20A, 22F, 23A, 23B, 24F, 31, 33F and 35B;
3, 6A, 7F, 8, 9N, 10A, 11A, 12F, 15A, de-O-acetylated-15B, 16F, 17F, 19A, 20A, 22F, 23A, 23B, 24F, 31, 33F and 35B;
3, 6A, 7F, 8, 9N, 10A, 11A, 12F, 15A, 15C, 16F, 17F, 19A, 20A, 22F, 23A, 23B, 24F, 31, 33F and 35B;
3, 6A, 7F, 8, 9N, 10A, 11A, 12F, 15A, 15B, 16F, 17F, 19A, 20, 22F, 23A, 23B, 24F, 31, 33F and 35B;
3, 6A, 7F, 8, 9N, 10A, 11A, 12F, 15A, de-O-acetylated-15B, 16F, 17F, 19A, 20, 22F, 23A, 23B, 24F, 31, 33F and 35B;
3, 6A, 7F, 8, 9N, 10A, 11A, 12F, 15A, 15C, 16F, 17F, 19A, 20, 22F, 23A, 23B, 24F, 31, 33F and 35B;
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B;
and the carrier protein is CRM197.

49. The composition of claim 48, wherein the SPAN-85 concentration is 6 μg/mL-14 mg/mL, the PS-20 or PS-80 concentration is 6 μg/mL-14 mg/mL, the squalene concentration is 60 μg/mL-34 mg/mL and the cationic lipid concentration is 30 μg/mL-2.4 mg/mL.

50. The composition of claim 48, wherein the SPAN-85 concentration is 6 μg/mL-14 mg/mL, the PS-20 or PS-80 concentration is 6 μg/mL-14 mg/mL, the squalene concentration is 60 μg/mL-34 mg/mL and the cationic lipid concentration is 60 μg/mL-2.4 mg/mL.

51. A composition comprising:
a) at least one Streptococcus pneumoniae polysaccharide-carrier protein conjugate comprising a Streptococcus pneumoniae polysaccharide conjugated to a carrier protein;
b) sorbitan trioleate (SPAN-85);
c) polysorbate-20 (PS-20);
d) squalene; and
e) a cationic lipid selected from:
(13Z,16Z)—N,N-dimethyl-3-nonyldocosa-13,16-dien-1-amine;
(6Z,9Z,26Z,29Z)—N,N-dimethylpentatriaconta-6,9,26,29-tetraen-18-amine; and
N,N-dimethyl-1-((1S,2R)-2-octylcyclopropyl)heptadecan-8-amine;
wherein the at least one Streptococcus pneumoniae polysaccharide is selected from the group consisting of serotypes:
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B;
and the carrier protein is CRM197.

52. The composition of claim 51, wherein the SPAN-85 concentration is 6 μg/mL-14 mg/mL, the PS-20 or PS-80 concentration is 6 μg/mL-14 mg/mL, the squalene concentration is 60 μg/mL-34 mg/mL and the cationic lipid concentration is 30 μg/mL-2.4 mg/mL.

53. The composition of claim 51, wherein the SPAN-85 concentration is 6 μg/mL-14 mg/mL, the PS-20 or PS-80 concentration is 6 μg/mL-14 mg/mL, the squalene concentration is 60 μg/mL-34 mg/mL and the cationic lipid concentration is 60 μg/mL-2.4 mg/mL.

54. A composition comprising:
a) at least one Streptococcus pneumoniae polysaccharide-carrier protein conjugate comprising a Streptococcus pneumoniae polysaccharide conjugated to a carrier protein;
b) sorbitan trioleate (SPAN-85);
c) polysorbate-20 (PS-20);
d) squalene; and
e) a cationic lipid which is (13Z,16Z)—N,N-dimethyl-3-nonyldocosa-13,16-dien-1-amine;
wherein the at least one Streptococcus pneumoniae polysaccharide is selected from the group consisting of serotypes:
3, 6A, 7F, 8, 9N, 10A, 11A, 12F, 15A, 15B, 16F, 17F, 19A, 20B, 22F, 23A, 23B, 24F, 31, 33F and 35B;
3, 6A, 7F, 8, 9N, 10A, 11A, 12F, 15A, de-O-acetylated-15B, 16F, 17F, 19A, 20B, 22F, 23A, 23B, 24F, 31, 33F and 35B; and
3, 6A, 7F, 8, 9N, 10A, 11A, 12F, 15A, 15C, 16F, 17F, 19A, 20B, 22F, 23A, 23B, 24F, 31, 33F and 35B;
and the carrier protein is CRM197.

55. The composition of claim 54, wherein the SPAN-85 concentration is 6 μg/mL-14 mg/mL, the PS-20 or PS-80 concentration is 6 μg/mL-14 mg/mL, the squalene concentration is 60 μg/mL-34 mg/mL and the cationic lipid concentration is 30 μg/mL-2.4 mg/mL.

56. The composition of claim 55 wherein the composition further comprises 5 mM-40 mM histidine at pH 5.1-7.0 and 25 mM-300 mM NaCl.

57. The composition of claim 55 wherein the composition further comprises about 20 mM histidine at about pH 5.8 and about 75 mM NaCl.

58. The composition of claim 54, wherein the SPAN-85 concentration is 6 μg/mL-14 mg/mL, the PS-20 or PS-80 concentration is 6 μg/mL-14 mg/mL, the squalene concentration is 60 μg/mL-34 mg/mL and the cationic lipid concentration is 60 μg/mL-2.4 mg/mL.

59. The composition of claim 58 wherein the composition further comprises 5 mM-40 mM histidine at pH 5.1-7.0 and 25 mM-300 mM NaCl.

60. The composition of claim 58 wherein the composition further comprises about 20 mM histidine at about pH 5.8 and about 75 mM NaCl.
